(19) United States
(12) Patent Application Publication
(10) Pub. No.: US 2025/0111899 A1
(43) Pub. Date: Apr. 3, 2025

(54) PREDICTING INSERT LENGTHS USING PRIMARY ANALYSIS METRICS

(71) Applicant: Illumina, Inc., San Diego, CA (US)

(72) Inventors: Gavin Derek Parnaby, Laguna Niguel, CA (US); Michael Ruehle, Fort Worth, TX (US); Rami Mehio, San Diego, CA (US); Jeffrey Fun-Shen Gau, San Mateo, CA (US); Jeffrey Yuan, Long Island City, NY (US)

(21) Appl. No.: 18/899,685

(22) Filed: Sep. 27, 2024

Related U.S. Application Data
(60) Provisional application No. 63/586,564, filed on Sep. 29, 2023.

(51) Int. Cl. G16B 30/20; G16B 40/10

(52) U.S. Cl. CPC G16B 30/20; G16B 40/10

(57) ABSTRACT

This disclosure describes embodiments of methods, non-transitory computer readable media, and systems that can utilize one or more machine learning models to predict insert lengths of a sample genomic sequence from which nucleotide read pairs are sequenced. For example, the disclosed systems can generate predictions for insert lengths based on cluster metrics from primary analysis on a sequencing device, such as signal intensity. By applying a machine-learning-based insert length prediction model to process the cluster metrics, the disclosed systems generate a predicted insert length (e.g., a distribution or a mean). To determine cluster metrics, the disclosed systems can analyze data from oligonucleotide clusters and/or from a sample genomic sequence used to sequence nucleotide read pairs during primary analysis. Based on predicted insert lengths from cluster metrics, the disclosed systems can determine improved genotype calls for genomic samples, such as calls in genomic regions comprising tandem repeats or structural variants.

### CROSS-REFERENCE TO RELATED APPLICATIONS

This application claims priority to and the benefit of U.S. Provisional Patent Application No. 63/586,564, entitled “PREDICTING INSERT LENGTHS USING PRIMARY ANALYSIS METRICS,” filed on Sep. 29, 2023, which is incorporated herein by reference in its entirety.

### BACKGROUND

In recent years, biotechnology firms and research institutions have improved hardware and software for performing secondary analysis of genomic samples, such as software for genotype calling based on nucleotide sequences of genomic samples. In particular, some existing sequencing platforms generate genotype calls from nucleotide reads of a genomic sample and/or run diagnostics on variant calls for a variety of purposes. For example, as part of primary analysis, some existing sequencing systems determine individual nucleotide bases (or “nucleobases”) within sequences by using conventional Sanger sequencing or by using sequencing-by-synthesis (SBS) methods. When using SBS, existing systems can monitor millions to billions of nucleic acid polymers being synthesized in parallel to predict nucleobase calls for nucleotide reads. For instance, a camera in many SBS platforms captures images of irradiated fluorescent tags incorporated into oligonucleotides for determining the nucleobase calls. After capturing such images, existing SBS platforms send call data (or image-based data) to a computing device to apply existing sequencing data analysis software as part of secondary analysis that determines a nucleobase sequence for a genomic sample or other nucleic acid polymer, such as a whole genome sequence for a genomic sample or variant calls for particular genomic regions. To facilitate such sequence determinations and variant calling, in some cases, the sequencing data analysis software either estimates an insert length of genomic deoxyribonucleic acid (DNA) in a library template or uses a mean of such an insert length. But existing system currently base such an insert-length estimate exclusively on metrics from secondary analysis, including metrics for mapping and aligning nucleotide reads and determining variant calls from aligned nucleotide reads.

Despite these recent advances, existing sequencing platforms and sequencing data analysis software (together and hereinafter, existing sequencing systems) continue to exhibit a number of drawbacks or disadvantages with respect to estimating and using insert length. For example, many existing sequencing systems generate inaccurate predictions of insert lengths for the genomic DNA in a given library template. Indeed, as mentioned, some existing sequencing systems utilize models to (i) identify nucleotide read pairs that have been mapped and aligned to non-repeat genomic regions of a reference genome and exhibit relatively high mapping quality (e.g., MAPQ 40) and (ii) generate a normal probability distribution of insert lengths by applying independent and identically distributed (IID) methods with a fixed mean and standard deviation to determine a distribution of genomic DNA fragments corresponding to such mapped and aligned nucleotide read pairs. While using such IID probability distributions provides some indication of insert length ranges, the predictions are imprecise, especially for repeat regions of genomic motifs or genomic regions corresponding to candidate structural variants-both of which can have many possible read mappings.

Consequently, existing sequencing systems that rely on a mean insert length based on metrics from exclusively secondary analysis sometimes produce flawed and inaccurate insert lengths, such as negative insert lengths (which are physically impossible) in cases where the insert length of a nucleotide read pair is relatively short. Because existing probability distributions for insert length are limited to data from nucleotide reads with relatively high mapping quality to non-repeat genomic regions, an existing mean insert length from such probability distributions have proven unreliable for more difficult-to-map genomic regions. Such difficult-to-map genomic regions include, for example, structural variants, variable number tandem repeat (VNTRs), short tandem repeat (STRs), segmental duplications, long interspersed nucleotide elements (LINEs), and short interspersed nucleotide elements (SINEs).

Not only do some existing sequencing systems inaccurately determine insert lengths, but the methods by which existing sequencing systems determine the insert lengths often render the insert lengths unreliable for certain types of secondary analysis, such as genomic mapping (e.g., mapping nucleotide reads to a reference genome) and/or genotype calling. For instance, because existing sequencing systems predict insert lengths by generating probability distributions based on metrics from secondary analysis, these systems cannot use predicted insert lengths based on metrics for the same sequencing run for later mapping or genotype calling and must initially rely on average insert lengths based on metrics from past secondary analyses.

Even when existing sequencing systems utilize predicted insert lengths from past secondary analyses to inform mapping or genotype calling, such systems nevertheless suffer from inaccuracies stemming from the inaccurate insert lengths discussed above. Especially in circumstances where two candidates have different insert lengths (and/or stemming from the inaccurate insert length predictions), existing sequencing systems can further produce inaccurate genotype calls due in part to inaccurate predicted insert lengths. Indeed, without more accurate methods to determine insert lengths for paired nucleotide reads, and without the ability to determine insert lengths before secondary analysis processes, such as mapping or variant calling, existing sequencing systems exhibit mapping and genotype calling performance that can be improved.

### SUMMARY

This disclosure describes embodiments of methods, non-transitory computer readable media, and systems that can utilize one or more machine learning models to predict insert lengths of a sample genomic sequence from which nucleotide read pairs are sequenced. For example, the disclosed systems can generate predictions for insert lengths based on cluster metrics from primary analysis on a sequencing device, such as signal intensity. By applying a machine-learning-based insert length prediction model to process the cluster metrics, the disclosed systems generate a distribution of predicted insert lengths of the sample genomic sequence or a mean predicted insert length from such a distribution of predicted insert lengths. To determine cluster metrics, the disclosed systems can analyze data from oligonucleotide clusters and/or from a sample genomic sequence used to sequence nucleotide read pairs during primary analysis. Based on predicted insert lengths from cluster metrics, the disclosed systems can determine improved genotype calls for genomic samples, such as calls in genomic regions comprising tandem repeats or structural variants. As further illustrated below, in some cases, the disclosed systems can leverage the predicted insert lengths to improve mapping of nucleotide read pairs to more accurate genomic regions of a reference genome.

### BRIEF DESCRIPTION OF THE DRAWINGS

The detailed description refers to the drawings briefly described below.

FIG. 1 illustrates a block diagram of a system environment including an insert length prediction system in accordance with one or more embodiments.

FIG. 2 illustrates an overview of generating a predicted insert length for mapping and genotype calling in accordance with one or more embodiments.

FIG. 3 illustrates an example graph of insert lengths determined by existing sequencing systems in accordance with one or more embodiments.

FIG. 4 illustrates a diagram for improved mapping based on more accurate insert length predictions in accordance with one or more embodiments.

FIG. 5 illustrates an example diagram for generating or determining cluster metrics in accordance with one or more embodiments.

FIG. 6 illustrates an example diagram for determining a cluster intensity metric in accordance with one or more embodiments.

FIG. 7 illustrates an example diagram for mapping and genotype calling based on a predicted insert length in accordance with one or more embodiments.

FIG. 8 illustrates example graphs representing the relationship between insert size and cluster intensity in accordance with one or more embodiments.

FIG. 9 illustrates an example training diagram for an insert length prediction model in accordance with one or more embodiments.

FIG. 10 illustrates an example diagram for determining a variant call for a variable number tandem repeat based on a predicted insert length in accordance with one or more embodiments.

FIG. 11 illustrates an example diagram for determining a variant call for a structural variant based on a predicted insert length in accordance with one or more embodiments.

FIG. 12 illustrates an example graph depicting anomalous insert length values on a per-cluster or per-fragment level in accordance with one or more embodiments.

FIG. 13 illustrates a flowchart of a series of acts for generating a predicted insert length for mapping and genotype calling in accordance with one or more embodiments.

FIG. 14 illustrates a block diagram of an example computing device for implementing one or more embodiments of the present disclosure.

### DETAILED DESCRIPTION

This disclosure describes embodiments of an insert length prediction system that can generate predicted insert lengths of a sample genomic sequence for nucleotide read pairs using a specialized insert length prediction model. To elaborate, the insert length prediction system can utilize an insert length prediction model to process cluster metrics associated with an oligonucleotide cluster and/or associated with a sample genomic sequence from the cluster to generate a predicted insert length of the sample genomic sequence based on the cluster metrics. For example, the insert length prediction system determines cluster metrics that indicate various attributes or measurements of a cluster of oligonucleotides within a well of a flow cell, within a random/non-patterned flow cell, within a complementary metal oxide semiconductor and/or other environments that sequence oligonucleotides (e.g., Complementary Metal-Oxide Semiconductor (CMOS) detection device or other sensors). In addition, the insert length prediction system can predict the insert length for the sample genomic sequence as a number of nucleobases that make up the genomic sequence by using an insert length prediction model to process the cluster metrics.

In certain implementations, the insert length prediction system can identify a sample genomic sequence from a cluster of oligonucleotides. For example, the insert length prediction system identifies a cluster from a well within a flow cell (e.g., a patterned flow cell or a non-patterned flow cell). In addition, in some embodiments, the insert length prediction system identifies or extracts a sample genomic sequence from the cluster. For instance, the insert length prediction system identifies a sample genomic sequence generated (e.g., at a particular cycle) as part of an SBS process for sequence synthesis and amplification.

In some cases, the insert length prediction system further identifies a nucleotide read pair for the sample genomic sequence. For instance, the insert length prediction system determines a first read in the pair that complements (e.g., indicates nucleobases of) a first portion of the sample genomic sequence extending in a first direction from the end of a first adapter sequence toward a second adapter sequence. In some cases, the insert length prediction system also determines a second read in the pair that complements (e.g., indicates nucleobases of) a second portion of the sample genomic sequence extending in a second direction from the end of the second adapter sequence toward the first adapter sequence. The corresponding sample genomic sequence includes at least the genomic DNA between the first and second adapter sequences.

As also mentioned, in one or more implementations, the insert length prediction system determines cluster metrics associated with an oligonucleotide cluster. For example, the insert length prediction system determines cluster metrics by analyzing or measuring characteristics of a cluster as part of primary sequencing analysis. In some cases, the insert length prediction system determines cluster metrics that are specific to an extracted/identified sample genomic sequence. In these or other cases, the insert length prediction system determines cluster metrics that correspond to (or are determined via analysis of) a cluster or well within a flow cell (e.g., a cluster/well that contains the sample genomic sequence).

Additionally, in certain embodiments, the insert length prediction system generates or predicts an insert length from the cluster metrics. For example, the insert length prediction system utilizes an insert length prediction model to generate at least one predicted insert length (e.g., a distribution of predicted insert lengths) of a sample genomic sequence by processing the cluster metrics of the sequence and/or the cluster (or well) where the sample genomic sequence was synthesized. Accordingly, the insert length prediction system can generate a predicted insert length of a sample genomic that reflects a predicted number of nucleobases between adapter ends of a DNA fragment synthesized during an SBS process.

In some embodiments, the insert length prediction system further performs mapping and genotype calling processes informed and improved by the insert length of a sample genomic sequence. For example, the insert length prediction system maps a nucleotide read pair to a genomic region of a reference genome based on insert length (and other factors, such as nucleobase similarity between the reference genome and the paired reads). In addition, the insert length prediction system can determine a genotype call for a genomic sequence based on the mapping. Accordingly, the insert length prediction system can improve secondary analysis processes based on insert length predicted based on primary analysis metrics (e.g., cluster metrics).

As just mentioned, the insert length prediction system can perform genotype calling based on predicting an insert length for a sample genomic sequence. For example, based on a predicted insert length (e.g., a distribution of predicted insert lengths), the insert length prediction system can generate or determine a variant call, such as a tandem repeat (e.g., a variable number tandem repeat or VNTR) or a structural variant. In some cases, the insert length prediction system generates or determines a tandem repeat, such as a VNTR, based on determining haplotype probabilities and corresponding haplotype lengths for one or more genomic coordinates of a sample genomic sequence. In these or other cases, the insert length prediction system generates or determines a structural variant by comparing a predicted insert length with an expected insert length for a sample genomic sequence to identify candidate structural variant coordinates.

As suggested above, the insert length prediction system can provide several advantages, benefits, and/or improvements over existing sequencing systems. For instance, the insert length prediction system introduces a first-of-its-kind insert length prediction model that performs new functions not available in existing sequencing systems by predicting insert lengths of genomic sequences based on cluster metrics from primary analysis. Indeed, while some existing systems can predict insert lengths based on current or past secondary analysis metrics to produce probability distributions, the insert length prediction system can generate predicted insert lengths from primary analysis metrics (e.g., cluster metrics) using an insert length prediction model. Thus, beyond capturing insert lengths using a new model, the insert length prediction system can determine insert lengths at a point in the genetic-sequencing workflow where the insert length informs and improves downstream processes, such as secondary analysis mapping and genotype calling.

In contrast to existing systems that sometimes generate erroneous insert length predictions (e.g., negative length values) based on faulty probability distributions, the insert length prediction system uses an insert length prediction model based on cluster metrics for more precise insert length predictions (e.g., using distributions that exclude negative value regions). For example, the insert length prediction system determines specific cluster metrics (e.g., signal intensity, cluster offset, and others) that inform the insert length prediction model to accurately predict an insert length for a genomic sequence, especially for repeat regions. Because the insert length prediction system utilizes primary metrics, such as cluster metrics, the insert length prediction system can further utilize the insert length prediction to inform and improve secondary analysis processes, such as mapping and genotype calling, as opposed to existing sequencing systems that cannot do so because they predict insert lengths as part of (or after) secondary analysis processes.

Due at least in part to the improved predictions of insert length, the insert length prediction system can further improve the accuracy of mapping or genotype calling. For example, the insert length prediction system can utilize a predicted insert length as a parameter or a metric for mapping nucleotide reads of a read pair to a reference genome. In some case, the insert length prediction system thus maps paired nucleotide reads more accurately than existing sequencing systems that cannot use insert length as a metric for the mapping process. For similar reasons, the insert length prediction system can further improve upon the accuracy of existing sequencing systems in generating genotype calls for a genomic sequence (e.g., determining genotype calls for genomic coordinates in relation to a reference genome).

For example, the insert length prediction system improves the accuracy of specific variant calls, such as tandem repeats (e.g., VNTRs) and structural variants, as a consequence of improving the accuracy in predicting insert lengths. Indeed, as set forth below, the insert length prediction system can determine tandem repeats and structural variants utilizing specific processes and algorithms which incorporate the predicted insert length of a sample genomic coordinate. As an example, the insert length prediction system improves over existing systems that miss candidate structural variant locations because of their inaccurate insert lengths which result in inconsistent and/or unreliable comparisons with expected insert lengths. By more accurately predicting insert lengths, the insert length prediction system can better compare predicted insert lengths with expected insert lengths to identify candidate coordinates of a sample genomic coordinate where a structural variant (e.g., an indel of at least a threshold number of nucleobases) might exist.

As suggested by the foregoing discussion, this disclosure utilizes a variety of terms to describe features and benefits of the insert length prediction system. Additional detail is hereafter provided regarding the meaning of these terms as used in this disclosure. As used in this disclosure, for instance, the term “sample genomic sequence” or “sample sequence” refers to a sequence of nucleotides isolated or extracted from a sample organism (or a copy of such an isolated or extracted sequence). In particular, a sample genomic sequence includes a segment of a nucleic acid polymer that is isolated or extracted from a sample organism and composed of nitrogenous heterocyclic bases. A sample genomic sequence can include a sequence of nucleobases generated or synthesized in a flow cell as part of a sequencing run of an SBS process. For example, a sample genomic sequence can include a segment of deoxyribonucleic acid (DNA), ribonucleic acid (RNA), or other polymeric forms of nucleic acids or chimeric or hybrid forms of nucleic acids noted below. More specifically, in some cases, the sample genomic sequence is found in a sample prepared or isolated by a kit and received by a sequencing device. In some embodiments, the nucleobases are known for the coordinates of the sample genomic sequence, while in other embodiments some or all of the nucleobases are unknown. For instance, a sample genomic sequence can include or refer to a genomic fragment that consists of a strand of nucleobases, where reads starting at either end of the strand identify the nucleobases at various coordinates (and where some nucleobases within an inner distance between the reads are unknown).

As further used herein, the term “genotype call” refers to a determination or prediction of a particular genotype of a genomic sample at a genomic locus. In particular, a genotype call can include a prediction of a particular genotype of a sample genomic sequence with respect to a reference genome at a genomic coordinate or a genomic region. For instance, in some cases, a genotype call includes a determination or a prediction that a genomic sample comprises both a nucleobase and a complementary nucleobase at a genomic coordinate that is either homozygous or heterozygous for a reference base or a variant (e.g., homozygous reference bases represented as 010 or heterozygous for a variant on a particular strand represented as 01i). Accordingly, a genotype call can include a prediction of a variant or reference base for one or more alleles of a genomic sample and indicate zygosity with respect to a variant or reference base. A genotype call is often determined for a genomic coordinate or genomic region at which an SNP, insertion, deletion, or other variant has been identified for a population of organisms.

Relatedly, as used herein, the term “nucleotide read” (or simply “read”) refers to an inferred or predicted sequence of one or more nucleotide bases (or nucleobase pairs) from all or part of a sample genomic sequence (e.g., a sample genomic sequence, complementary DNA). In particular, a nucleotide read includes a determined or predicted sequence of nucleobase calls for a nucleotide fragment (or group of monoclonal nucleotide fragments) from a sequencing library corresponding to a genomic sample. For example, in some embodiments, the insert length prediction system determines a nucleotide read by generating nucleobase calls for nucleobases passed through a nanopore of a nucleotide-sample slide, determined via fluorescent tagging, or determined from a well in a flow cell. In some cases, a nucleotide read can refer to a particular type of read, such as a nucleotide read synthesized from sample library fragments that are shorter than a threshold number of nucleobases (e.g., SBS reads). In these or other cases, another type of nucleotide read can refer to (i) assembled nucleotide reads that have been assembled from shorter nucleotide reads to form a contiguous sequence (e.g., assembled nucleotide reads) satisfying a threshold number of nucleobases, (ii) circular consensus sequencing (CCS) reads satisfying the threshold number of nucleobases, or (iii) nanopore long reads satisfying the threshold number of nucleobases.

Relatedly, as used herein, the term “nucleotide read pair” refers to a pair of nucleotide reads that are associated with, or read from, a shared genomic sequence. For example, a nucleotide read pair includes a first read and a second read that each indicate nucleobase calls for nucleotides at various coordinates of a sample genomic sequence synthesized from a cluster of oligonucleotides. In some cases, a nucleotide read pair includes: i) a first nucleotide read starting at a first endpoint of an insert (e.g., at the end of an adapter sequence on a first end) and extending in a first direction toward the opposite endpoint of the insert and ii) a second nucleotide read starting at the opposite endpoint of the insert (e.g., at the end of adapter sequence on a second end) and extending in a second direction toward the first endpoint.

Along these lines, as used herein, an “insert” refers to a sample genomic sequence that spans, or extends between, a first adapter sequence on one end of the sample genomic sequence and a second adapter sequence on another of the sample genomic sequence. For example, an insert can include a sequence of genomic or transcriptomic DNA extracted from a genomic sample that extends between one surface-bound oligonucleotide (e.g., a binding adapter sequence) bound to a well within a nucleotide-sample slide (e.g., a nanowell of a flow cell) and another surface-bound oligonucleotide (e.g., a binding adapter sequence) bound to the well within the nucleotide-sample slide. In some cases, an insert includes priming and indexing sequences on either end of the sequence, while in other cases an insert excludes priming and indexing sequences.

Relatedly, the term “insert length” (or “insert size”) refers to a number of nucleobases included within, or that make up, an insert. For example, an insert length includes a number of nucleobases that constitute the insert spanning between adapter sequences of a sample genomic sequence. In some cases, an insert (or a fragment) includes an entire nucleobase strand or sequence attached to a flow cell, including adapter sequences on both ends and the insert therebetween. However, in some cases, adapter sequences are small enough that, statistically or as a model choice, an insert length can include adapter sequences (e.g., priming sequences, index sequences, and/or binding adapter sequences). In one or more embodiments, at least an insert length or at least a predicted insert length includes or refers to a distribution of insert lengths, such as a parametric distribution per sample of predicted insert lengths, a non-parametric distribution of predicted insert lengths, a quantile of predicted insert lengths, an expectile of predicted insert lengths, or a mean and standard deviation of predicted insert lengths.

As further used herein, the term “nucleotide-sample slide” (or “nucleotide-sample substrate”) refers to a plate or substrate, such as a flow cell, comprising oligonucleotides for sequencing nucleotide sequences from genomic samples or other sample nucleic-acid polymers. In particular, a flow cell can refer to a substrate containing fluidic channels through which reagents and buffers can travel as part of sequencing. For example, in one or more embodiments, the flow cell (e.g., a patterned flow cell or non-patterned flow cell) may comprise small fluidic channels and oligonucleotide samples that can be bound to adapter sequences on the substrate. In other implementations, a flow cell can be an open substrate with one or more regions for oligonucleotide samples to be analyzed and the oligonucleotide samples may be positioned using charged pads or other means. In yet another implementation, the nucleotide-sample substrate can be a membrane having a nanopore through which one or more oligonucleotide samples may pass. As indicated above, a flow cell can include tiles and wells (e.g., nanowells) comprising clusters of oligonucleotides.

As used herein, a flow cell or other nucleotide-sample slide can (i) include a device having a lid extending over a reaction structure to form a flow channel therebetween that is in communication with a plurality of reaction sites of the reaction structure and (ii) include a detection device that is configured to detect designated reactions that occur at or proximate to the reaction sites. A flow cell or other nucleotide-sample slide may include a solid-state light detection or “imaging” device, such as a Charge-Coupled Device (CCD) or Complementary Metal-Oxide Semiconductor (CMOS) (light) detection device. As one specific example, a flow cell may be configured to fluidically and electrically couple to a cartridge (having an integrated pump), which may be configured to fluidically and/or electrically couple to a bioassay system. A cartridge and/or bioassay system may deliver a reaction solution to reaction sites of a flow cell according to a predetermined protocol (e.g., sequencing-by-synthesis), and perform a plurality of imaging events. For example, a cartridge and/or bioassay system may direct one or more reaction solutions through the flow channel of the flow cell, and thereby along the reaction sites. At least one of the reaction solutions may include four types of nucleotides having the same or different fluorescent labels. The nucleotides may bind to the reaction sites of the flow cell, such as to corresponding oligonucleotides at the reaction sites. The cartridge and/or bioassay system may then illuminate the reaction sites using an excitation light source (e.g., solid-state light sources, such as light-emitting diodes (LEDs)). The excitation light may provide emission signals (e.g., light of a wavelength or wavelengths that differ from the excitation light and, potentially, each other) that may be detected by the light sensors of the flow cell.

In addition, as used herein, the term “cluster of oligonucleotides” (or simply “cluster”) refers to a localized group or collection of DNA or RNA molecules on a nucleotide-sample slide, such as a flow cell, or other solid surface. In particular, a cluster includes tens, hundreds, thousands, or more copies of a cloned or the same DNA or RNA segment. For example, in one or more embodiments, a cluster includes a grouping of oligonucleotides immobilized in a section of a flow cell or other sample slide. In some embodiments, clusters are evenly spaced or organized in a systematic structure within a patterned flow cell. By contrast, in some cases, clusters are randomly organized within a non-patterned flow cell. A cluster of oligonucleotides can be imaged utilizing one or more light signals. For instance, an oligonucleotide-cluster image may be captured by a camera during a sequencing cycle of light emitted by irradiated fluorescent tags incorporated into oligonucleotides from one or more clusters on a flow cell.

Relatedly, the term “cluster metric” refers to a metric, a measurement, or a parameter that is generated or determined based on analysis of one or more molecules of a cluster of oligonucleotides. For example, a cluster metric includes a parameter determined via one or more cycles of a sequencing run. In some embodiments, a cluster metric includes a metric determined via one or more primary analysis processes, where the metric is parameterized (e.g., represented mathematically or otherwise) for input into a machine learning model to generate a predicted insert length. For example, a cluster metric includes, but is not limited to, a cluster intensity metric corresponding to a cluster of oligonucleotides, a cluster offset metric indicating a signal intensity corresponding to the cluster of oligonucleotides in a non-luminescent state, or a signal-to-noise ratio (SNR) differential metric indicating a difference between an SNR for a first nucleotide read in a nucleotide read pair and an SNR for a second nucleotide read in a nucleotide read pair. Other example cluster metrics are described in detail below.

Along these lines, as used herein, the term “oligonucleotide” refers to an oligomer or other polymer of nucleotides or mimetics (e.g., complementary sequence). In particular, an oligonucleotide can include a synthetic or natural molecule comprising a sequence of covalently linked nucleotides formed by a modified phosphodiester or phosphodiester bond between the 3′ position of the pentose in a nucleotide and the 5′ position of the pentose in a nucleotide adjacent. For example, an oligonucleotide can include a short DNA or RNA molecule annealed to a single-stranded polynucleotide to be analyzed or sequenced as part of SBS sequencing.

As used herein, the term “sequencing run” refers to an iterative process on a sequencing device to determine a primary structure of nucleotide sequences from a sample (e.g., genomic sample). In particular, a sequencing run includes cycles of sequencing chemistry and imaging performed by a sequencing device (including an imaging device, such as a CCD or CMOS) that incorporate nucleobases into growing oligonucleotides to determine nucleotide reads from nucleotide sequences extracted from a sample (or other sequences within a library fragment) and seeded throughout a flow cell. In some cases, a sequencing run includes replicating oligonucleotides derived or extracted from one or more genomic samples seeded in clusters throughout a flow cell. Upon completing a sequencing run, a sequencing device can generate base-call data in a file, such as a binary base call (BCL) sequence file or a fast-all quality (FASTQ) file.

As used herein, the term “sequencing cycle” (or “cycle”) refers to an iteration of adding or incorporating one or more nucleobases to one or more oligonucleotides representing or corresponding to sample's sequence (e.g., a genomic or transcriptomic sequence from a sample) or a corresponding adapter sequence. In some cases, a sequencing cycle includes an iteration of both incorporating nucleobases into clusters of oligonucleotides using sequencing chemistry and capturing images of such clusters attached to a nucleotide-sample slide (e.g., a flow cell). Accordingly, cycles can be repeated as part of sequencing a nucleic-acid polymer (e.g., a sample genomic sequence). For example, in one or more embodiments, each sequencing cycle involves incorporating nucleobases into either a single nucleotide read in which DNA or RNA strands are read in only a single direction or paired-end reads in which DNA or RNA strands are read from both ends but in different cycles. Further, in certain cases, each sequencing cycle involves a camera taking an image of the nucleotide-sample slide or multiple sections of the nucleotide-sample slide to generate image data for determining a particular nucleotide base added or incorporated into particular oligonucleotides. Following the image capture stage, a sequencing system can remove certain fluorescent labels from incorporated nucleotide bases and perform another sequencing cycle until the nucleic-acid polymer has been completely sequenced. In one or more embodiments, a sequencing cycle includes a cycle within an SBS run. A sequencing cycle can include one or both of an indexing cycle and a genomic sequencing cycle. For instance, one cluster of oligonucleotides or a set of clusters of oligonucleotides may be undergoing a genomic sequencing cycle in which nucleobases corresponding to a sample genomic sequence are incorporated and another cluster of oligonucleotides or another set of clusters of oligonucleotides may be concurrently undergoing an indexing cycle in which nucleobases corresponding to an indexing sequence for a nucleotide read are incorporated.

As used herein, the term “reference genome” refers to a digital nucleic acid sequence assembled as a representative example (or representative examples) of genes and other genetic sequences of an organism. Regardless of the sequence length, in some cases, a reference genome represents an example set of genes or a set of nucleic acid sequences in a digital nucleic acid sequenced determined by scientists as representative of an organism of a particular species. For example, a linear human reference genome may be GRCh38 or other versions of reference genomes from the Genome Reference Consortium. As a further example, a reference genome may include a reference graph genome that includes both a linear reference genome and paths representing nucleic acid sequences from ancestral haplotypes, such as Illumina DRAGEN Graph Reference Genome hg19.

Further, as used herein, the term “variant call file” refers to a particular genotype-call data file that comprises a text file format that contains information about variants at specific genomic coordinates. For instance, a variant call file can include meta-information lines, a header line, and data lines where each data line contains information about a single genotype call (e.g., a single variant). As described further below, the dual-variant-type call recalibration system can generate different versions of genotype-call data files, including a pre-filter variant call file comprising variant genotype calls that either pass or fail a quality filter for base-call-quality metrics or a post-filter variant call file comprising variant genotype calls that pass the quality filter but excludes variant genotype calls that fail the quality filter.

In addition, as used herein, the term “tandem repeat” refers to a motif, a k-mer, or a pattern of one or more nucleotides in DNA or RNA that is repeated consecutively one motif, k-mer, or pattern of nucleotides after another. A tandem repeat can include minisatellites in which 10 to 60 nucleotides are repeated as part of a pattern. By contrast, a tandem repeat can also include microsatellites or short tandem repeats in which less than ten nucleotides are repeated as part of a pattern. To illustrate, an example tandem repeat includes a sequence of TAAGC TAAGC TAAGC in which the sequence TAAGC is repeated three times. To further illustrate, a tandem repeat may also include dinucleotide repeats (e.g., GCGCGCGC) and trinucleotide repeats (e.g., CAGCAGCAGCAG).

Relatedly, the term “variable number tandem repeat” or “VNTR” refers to a sequence of DNA at a genomic region comprising a tandem repeat and for which a population of genomic samples exhibit variation. In some cases, a population exhibits variations in length of nucleotide repeat units at a particular VNTR region. Accordingly, a VNTR can act as an inherited allele. As related to tandem repeats, the term “nucleotide repeat unit” (or simply “repeat unit”) refers to a single k-mer, motif, or unit of nucleotides within a pattern of nucleic acids that occur in multiple copies. In particular, a nucleotide repeat unit refers to a sequence of nucleic acids arranged next to at least one other identical sequence within a microsatellite, a minisatellite, or other tandem repeat. For example, a nucleotide repeat unit may be represented by an encoded nucleotide sequence, such as CGG or ATTCG.

As further used herein, the term “haplotype probability” refers to a probability or a score reflecting or evaluating a likelihood that a genomic sample or sample genomic sequence exhibits a haplotype (from among a set of candidate haplotypes). For example, a haplotype probability includes or reflects a likelihood that a haplotype having a certain length (or number of nucleobases) matches or corresponds to a particular region (e.g., a repeat region) of a sample genomic sequence, such as a fragment or a sample genomic sequence. Different haplotypes can have different haplotype probabilities that their respective lengths (or nucleobases) match or correspond to a genomic coordinate or region of a genomic sequence.

Additionally, the term “overlapping insert” refers to an insert (e.g., a sample genomic sequence or a portion of a sample genomic sequence) that overlaps or includes at least a portion of a repeat region (e.g., a region of a genomic sequence that includes repeat units). Relatedly, as used herein, the term “spanning insert” refers to an overlapping insert that has at least a threshold number of nucleobases on each side of a repeat region included within the insert. The term “flanking insert” refers to an overlapping insert that includes at least a threshold number of nucleobases on one side of a repeat region but not on the other side. The term “internal insert” refers to an overlapping insert that is entirely within a repeat region or that has less than a threshold number of nucleobases on both sides of the repeat region.

Further, as used herein, the term “structural variant” refers to a variation (e.g., deletion, insertion, translocation, inversion) in a structure of an organism's chromosome or a variation to nucleotide sequences of the organism's chromosome (e.g., a sample genomic sequence). In some cases, a structural variant includes a variation to a threshold number of base pairs (e.g., >50 base pairs) within an organism's chromosome. Accordingly, in certain implementations, a structural variant includes an insertion or deletion exceeding a threshold number of base pairs, a duplication exceeding a threshold number of base pairs, an inversion, a translocation, or a copy number variation (CNV). While some examples of structural variants use 50 base pairs as a threshold number of base pairs, in some embodiments, the threshold number of base pairs for a structural variant may be different, such as 16, 25, 32, 35, 45, 100, or 1,000 base pairs.

Additionally, a “fragment probability” refers to a probability or a composite numeric score evaluating or reflecting a likelihood of a nucleotide-read fragment supporting a given allele. In particular, a fragment probability includes a metric indicating a degree to which the nucleobases of a nucleotide-read fragment match, or are similar to, a given allele (e.g., from among a set of candidate alleles).

As suggested above, the insert length prediction system can utilize one or more machine learning models to generate predicted insert lengths of sample genomic sequences. As used herein, the term “machine learning model” refers to a computer algorithm or a collection of computer algorithms that automatically improve for a particular task through experience based on use of data. For example, a machine-learning model can utilize one or more learning techniques to improve in accuracy and/or effectiveness. Example machine-learning models include various types of decision trees, logistic regressions, linear regressions, random forests, support vector machines, Bayesian networks, or neural networks.

Relatedly, as used herein, the term “insert length prediction model” refers to a machine learning model that generates a predicted insert length for a sample genomic sequence. For example, an insert length prediction model includes a machine learning model that generates a predicted insert length by analyzing or processing one or more cluster metrics. In some cases, an insert length prediction model takes the form of an ensemble of gradient-boosted trees (e.g., XGBoost).

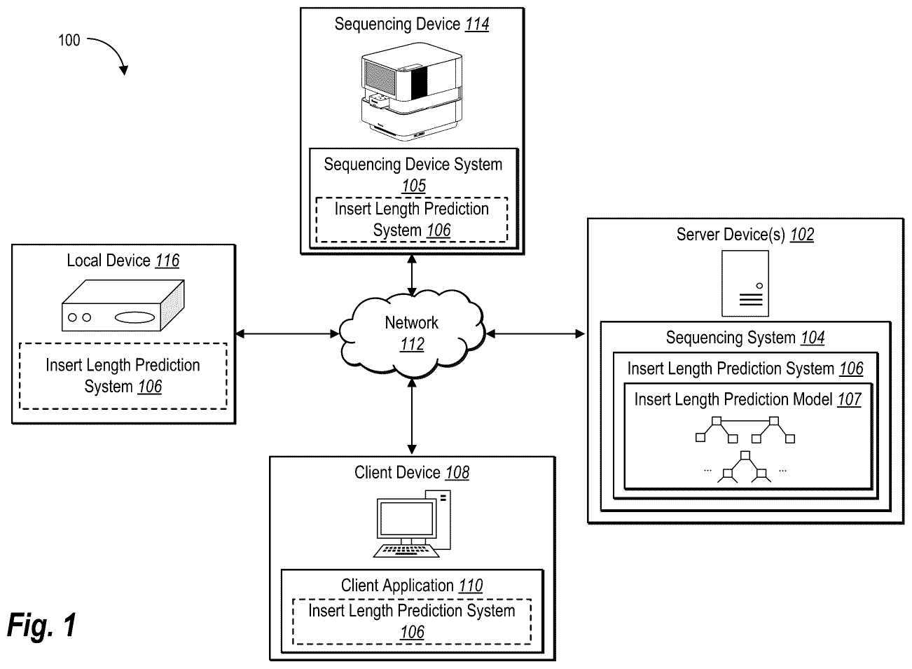

The following paragraphs describe the insert length prediction system with respect to illustrative figures that portray example embodiments and implementations. For example, FIG. 1 illustrates a schematic diagram of a system environment (or “environment”) 100 in which an insert length prediction system 106 operates in accordance with one or more embodiments. As illustrated, the environment 100 includes one or more server device(s) 102 connected to a client device 108, a local device 116, and a sequencing device 114 via a network 112. While FIG. 1 shows an embodiment of the insert length prediction system 106, this disclosure describes alternative embodiments and configurations below.

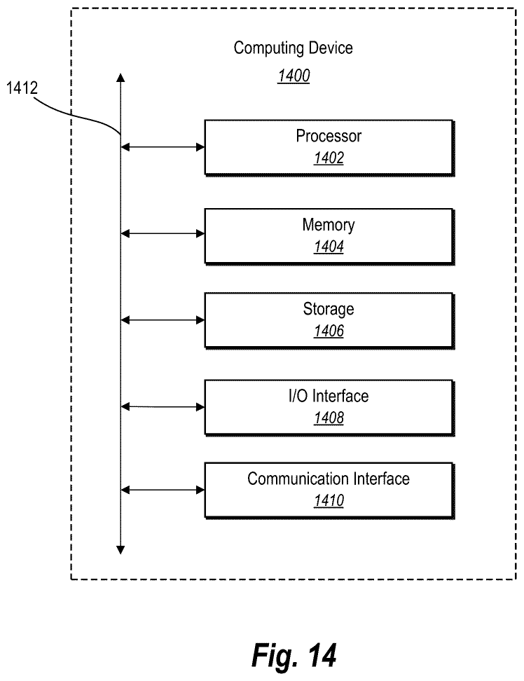

As shown in FIG. 1, the server device(s) 102, the client device 108, the local device 116, and the sequencing device 114 can communicate with each other via the network 112. The network 112 comprises any suitable network over which computing devices can communicate. Example networks are discussed in additional detail below with respect to FIG. 14.

As indicated by FIG. 1, the sequencing device 114 comprises a device for sequencing a nucleic acid polymer. In some embodiments, the sequencing device 114 analyzes nucleic acid segments or oligonucleotides extracted from genomic samples to generate nucleotide reads or other data utilizing computer implemented methods and systems (described herein) either directly or indirectly on the sequencing device 114. More particularly, the sequencing device 114 receives and analyzes, within nucleotide-sample slides (e.g., flow cells), nucleic acid sequences extracted from genomic samples. In one or more embodiments, the sequencing device 114 utilizes SBS to sequence nucleic acid polymers into nucleotide reads. In some embodiments, the sequencing device 114 generates one or more cluster metrics or cluster of oligonucleotides as part of an SBS process. In addition, or in the alternative to communicating across the network 112, in some embodiments, the sequencing device 114 bypasses the network 112 and communicates directly with the client device 108.

As further indicated by FIG. 1, the local device 116 is located at or near a same physical location of the sequencing device 114. Indeed, in some embodiments, the local device 116 and the sequencing device 114 are integrated into a shared/common computing device. The local device 116 may run the insert length prediction system 106 to generate, receive, analyze, store, and transmit digital data, such as by receiving cluster metrics or determining insert lengths, genotype calls, and/or variant calls based on analyzing such cluster metrics. As shown in FIG. 1, the sequencing device 114 may send (and the local device 116 may receive) cluster metrics generated during a sequencing run of the sequencing device 114. By executing software in the form of the insert length prediction system 106, the local device 116 may utilize an insert length prediction model 107 to generate predicted insert lengths based on the cluster metrics and may further perform mapping and genotype calling processes. The local device 116 may also communicate with the client device 108. In particular, the local device 116 can send data to the client device 108, including a variant call file (VCF), cluster metrics, or other information indicating nucleobase calls, mapping data, genotype calls, variant calls, error data, or other information.

As further indicated by FIG. 1, the server device(s) 102 may generate, receive, analyze, store, and transmit digital data, such as data for sequencing nucleic acid polymers, predicting insert lengths, mapping nucleotide reads, and/or generating genotype calls. As shown in FIG. 1, the sequencing device 114 may send (and the server device(s) 102 and/or the local device 116 may receive) call data and/or cluster metrics. The server device(s) 102 may also communicate with the client device 108 and/or the local device 116. In particular, the server device(s) 102 and/or the local device 116 can send data to the client device 108, including a variant call file or other information indicating nucleobase calls, mapping data, genotype calls, variant calls, cluster metrics, error data, or other information.

In some embodiments, the server device(s) 102 comprise a distributed collection of servers where the server device(s) 102 include a number of server devices distributed across the network 112 and located in the same or different physical locations. Further, the server device(s) 102 can comprise a content server, an application server, a communication server, a web-hosting server, or another type of server. In some cases, the server device(s) 102 are located at a same physical location as the sequencing device 114 and/or the local device 116.

As shown in FIG. 1, the sequencing device 114 includes a sequencing device system 105 for sequencing a genomic sample or other nucleic-acid polymer. In some embodiments, by executing the sequencing device system 105, the sequencing device 114 analyzes nucleic-acid segments or oligonucleotides extracted from genomic samples to generate nucleotide reads, cluster metrics, or other data utilizing computer implemented methods and systems (described herein) either directly or indirectly on the sequencing device 114. More particularly, the sequencing device 114 receives or analyzes nucleotide-sample slides (e.g., flow cells) comprising nucleotide sequences extracted from samples and then copies and determines the nucleobase sequence of such extracted nucleotide sequences. Indeed, by using the sequencing device system 105, the sequencing device 114 can run one or more sequencing cycles as part of a sequencing run to determine nucleobase sequences for nucleic acid polymers. In one or more embodiments, the sequencing device 114 utilizes Sequencing by Synthesis (SBS) to sequence nucleic-acid polymers into nucleotide reads.

As further shown in FIG. 1, the server device(s) 102 can include a sequencing system 104. Generally, the sequencing system 104 analyzes read data and/or cluster metrics received from the sequencing device 114 to map reads, generate genotype calls, and/or to generate variant calls. For example, the sequencing system 104 can receive read data and cluster metrics from the sequencing device 114 and can align and map nucleotides reads to genomic reads of a reference genome based on the cluster metrics. In some embodiments, the sequencing system 104 further determines a genotype call from the mapping and/or cluster metrics. In addition to processing sequences for nucleic acid polymers for mapping and genotype calling, the sequencing system 104 also generates a variant call file indicating one or more genotype calls and/or variant calls for one or more genomic coordinates.

As just mentioned, and as illustrated in FIG. 1, the insert length prediction system 106 analyzes cluster metrics (e.g., from the sequencing device 114 and/or from other processes performed by the sequencing system 104), to determine a predicted insert length for a sample genomic sequence. As shown, the insert length prediction system 106 includes an insert length prediction model 107. In some embodiments, the insert length prediction system 106 determines cluster metrics for sample genomic sequences. Based on data derived or prepared from the cluster metrics, the insert length prediction system 106 trains and/or applies an insert length prediction model 107 to generate a predicted insert length for a genomic sequence. In some cases, the insert length prediction system 106 further maps nucleotide reads and generates genotype calls based on the predicted insert length. Based on such data, for example, the insert length prediction system 106 can update data fields corresponding to a variant call file to update a genotype call and/or a variant call for improved accuracy.

As further illustrated and indicated in FIG. 1, the client device 108 can generate, store, receive, and send digital data. In particular, the client device 108 can receive cluster metrics from the sequencing device 114. Furthermore, the client device 108 may communicate with the server device(s) 102 and/or the local device 116 to receive a variant call file comprising genotype calls and/or other data, such as a call quality and/or a genotype quality. The client device 108 can accordingly present or display information pertaining to the genotype call within a graphical user interface to a user associated with the client device 108. For example, the client device 108 can present, via the client application 110, a graphical user interface that includes a visualization or a depiction of an insert length and/or a genotype call. In some cases, the client device can present, via the client application 110, a graphical user interface that includes or portrays various contribution measures associated with, or attributed to, individual cluster metrics with respect to a particular insert length.

The client device 108 illustrated in FIG. 1 may comprise various types of client devices. For example, in some embodiments, the client device 108 includes non-mobile devices, such as desktop computers or servers, or other types of client devices. In yet other embodiments, the client device 108 includes mobile devices, such as laptops, tablets, mobile telephones, or smartphones. Additional details regarding the client device 108 are discussed below with respect to FIG. 14.

As further illustrated in FIG. 1, the client device 108 includes a client application 110. The client application 110 may be a web application or a native application stored and executed on the client device 108 (e.g., a mobile application, desktop application). The client application 110 can include instructions that (when executed) cause the client device 108 to receive data from the insert length prediction system 106 and present, for display at the client device 108, data from a variant call file. Furthermore, the client application 110 can instruct the client device 108 to display a visualization of contribution measures for cluster metrics of an insert length.

As further illustrated in FIG. 1, the insert length prediction system 106 may be located on the client device 108 as part of the client application 110 or on the sequencing device 114 or on the local device 116. Accordingly, in some embodiments, the insert length prediction system 106 is implemented by (e.g., located entirely or in part) on the client device 108. In yet other embodiments, the insert length prediction system 106 is implemented by one or more other components of the environment 100, such as the sequencing device 114 or the local device 116. In particular, the insert length prediction system 106 can be implemented in a variety of different ways across the server device(s) 102, the network 112, the client device 108, and the sequencing device 114. For example, the insert length prediction system 106 can be downloaded from the server device(s) 102 to the client device 108, to the local device 116, and/or to the sequencing device 114 where all or part of the functionality of the insert length prediction system 106 is performed at each respective device within the environment 100.

Though FIG. 1 illustrates the components of environment 100 communicating via the network 112, in certain implementations, the components of environment 100 can also communicate directly with each other, bypassing the network 112. For instance, and as previously mentioned, in some implementations, the client device 108 communicates directly with the sequencing device 114 and/or the local device 116. Additionally, in some embodiments, the client device 108 communicates directly with the insert length prediction system 106 (hosted on one or more of the illustrated components). Moreover, the insert length prediction system 106 can access one or more databases housed on or accessed by the server device(s) 102 or elsewhere in the environment 100.

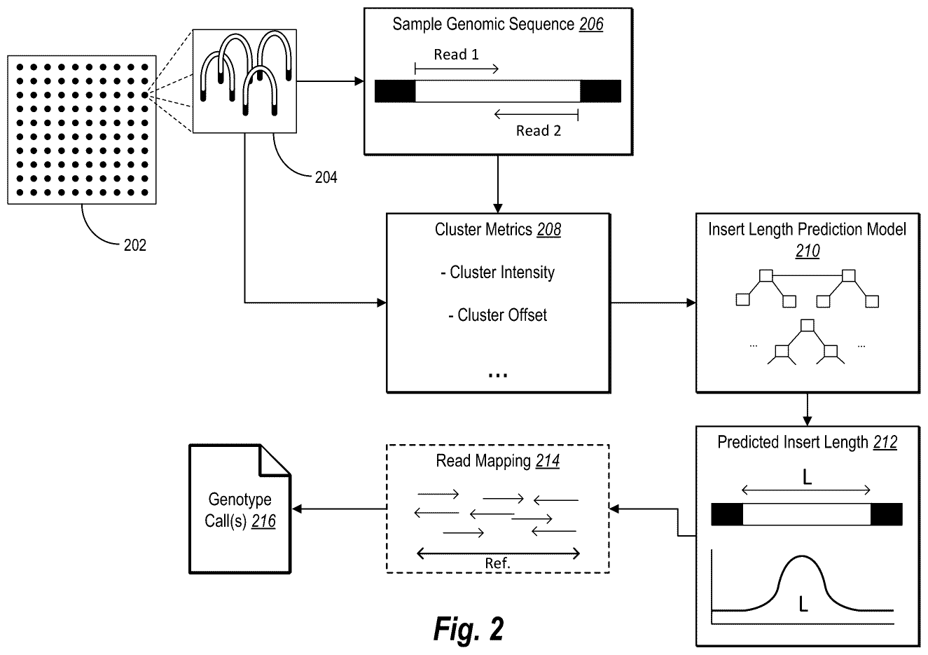

As indicated above, the insert length prediction system 106 can generate a predicted insert length for a sample genomic sequencing. In particular, the insert length prediction system 106 can predict an insert length using an insert length prediction model to process cluster metrics determined via a sequencing run. FIG. 2 illustrates an example overview for generating a predicted insert length using an insert length prediction model in accordance with one or more embodiments. Additional detail regarding the various acts and processes of FIG. 2 is provided thereafter with reference to subsequent figures.

As illustrated in FIG. 2, the insert length prediction system 106 determines, generates, detects, receives, or identifies data from a nucleotide-sample slide 202. To conserve space and depict clusters, FIG. 2 depicts only a region of the nucleotide-sample slide 202. In particular, the insert length prediction system 106 receives or determines primary sequencing data from a sequencing device that analyzes oligonucleotide clusters of the nucleotide-sample slide 202. In some cases, the insert length prediction system 106 performs a sequencing run (e.g., within one or more cycles of a sequencing run) to determine or generate sequencing data. In some embodiments, the insert length prediction system 106 receives or generates the sequencing data from the nucleotide-sample slide 202 in the form of cluster metrics 208.

To elaborate, the insert length prediction system 106 generates one or more of the cluster metrics 208 by analyzing a particular cluster or well of the nucleotide-sample slide 202. For instance, the insert length prediction system 106 determines or identifies an oligonucleotide cluster 204 to analyze for determining cluster metrics. In some cases, the insert length prediction system 106 generates cluster metrics 208, such as a signal intensity, a cluster offset, and others based on one or more sequencing processing during a sequencing run (e.g., over one or more cycles) for synthesize nucleobase sequences of the oligonucleotide cluster 204. While the insert length prediction system 106 determines some of the cluster metrics 208 from analysis of the oligonucleotide cluster 204 within the nucleotide-sample slide 202 (e.g., at the cluster level), in some embodiments, the insert length prediction system 106 determines some of the cluster metrics 208 from a specific sample genomic sequence within the oligonucleotide cluster 204 (e.g., at the sequence level).

To this point, as further illustrated in FIG. 2, the insert length prediction system 106 synthesizes, identifies, or extracts a sample genomic sequence 206 from the oligonucleotide cluster 204 (e.g., from a particular well or cluster within the nucleotide-sample slide 202). Indeed, as part of a sequencing run, the insert length prediction system 106 synthesizes and binds nucleobases on one another to generate the oligonucleotide cluster 204 which includes the sample genomic sequence. In some cases, the sample genomic sequence 206 includes adapter sequences (e.g., sequences of nucleobases for binding to the nucleotide-sample slide 202 and/or for other sequencing purposes) on either end (as represented by the black portions) and also includes an insert between the adapter sequences (represented by the white portion in the middle). In other cases, the sample genomic sequence 206 refers to an insert between adapter sequences and may or may not include priming sequences and/or indexing sequences.

As further shown in FIG. 2, the insert length prediction system 106 identifies or generates a nucleotide read pair for the sample genomic sequence 206. To elaborate, the insert length prediction system 106 identifies or generates a first nucleotide read (“Read 1”) and a second nucleotide read (“Read 2”) for the insert portion of the sample genomic sequence. Indeed, the insert length prediction system 106 determines a nucleotide read pair that includes Read 1 and Read 2, where Read 1 starts at one end of the insert and extends in a first direction (complementing a first portion of the sample genomic sequence 206) and Read 2 starts at the opposite end of the insert and extends in a second direction opposite the first direction (complementing a second portion of the sample genomic sequence 206).

As mentioned, in some embodiments, the insert length prediction system 106 generates one or more of the cluster metrics 208 from the sample genomic sequence 206. Examples of the cluster metrics 208 include, but are not limited to, a signal-to-noise ratio (“SNR”) differential metric, a guanine-cytosine (“GC”) content metric, and other cluster metrics 208 from the sample genomic sequence 206. Indeed, the insert length prediction system 106 determines the cluster metrics 208 at the sequence level that are specific to the sample genomic sequence 206 and further determines the cluster metrics 208 that are extracted/determined at the cluster level from the oligonucleotide cluster 204. Additional detail regarding and examples of the cluster metrics 208 and their determination is provided below with reference to subsequent figures.

As further illustrated in FIG. 2, the insert length prediction system 106 utilizes the cluster metrics 208 to generate at least a predicted insert length 212 (“L”), represented in FIG. 2 as a probability distribution of predicted insert lengths for some embodiments. More specifically, the insert length prediction system 106 inputs the cluster metrics 208 into an insert length prediction model 210 to generate the predicted insert length 212. For example, the insert length prediction model 210 processes the cluster metrics 208 using an ensemble of gradient-boosted trees which includes a series of weak learners, such as non-linear decision trees trained in a logistic regression, to generate the predicted insert length 212 in the form of a distribution of possible insert lengths (e.g., a parametric or non-parametric distribution) that indicates probabilities associated with respective lengths (e.g., including quantiles and/or a mean with one or more standard deviations of insert lengths).

Indeed, based on the cluster metrics 208, the insert length prediction system 106 generates the predicted insert length 212 which reflects or indicates a number of nucleobases of the insert within the sample genomic sequence 206 (e.g., excluding adapter sequences, priming sequences, and/or indexing sequences). In some cases, the predicted insert length 212 indicates a number of nucleobases for the entire insert or the entire sample genomic sequence 206, including the appended adapter sequences (and/or priming sequences and/or indexing sequences). In certain embodiments, the insert length prediction system 106 generates the predicted insert length 212 as an average of insert lengths for sequences within the oligonucleotide cluster 204.

In some embodiments, the insert length prediction system 106 generates the predicted insert length 212 in the form of a distribution of insert lengths, including multiple possible insert lengths corresponding to respective probabilities of corresponding to the sample genomic sequence 206. For instance, the insert length prediction system 106 can determine a parametric distribution of insert lengths or a non-parametric distribution of insert lengths. In certain cases, the insert length prediction system 106 determines that the predicted insert length 212 falls within a particular quantile of a parametric or non-parametric distribution. In some embodiments, the insert length prediction system 106 determines a mean insert length defined by an expected insert length value and one or more standard deviations (from the expected value).

In certain embodiments, the insert length prediction system 106 determines the predicted insert length 212 based on data from repeat regions of the sample genomic sequence 502. For instance, the insert length prediction system 106 determines a length (e.g., a number of nucleobases) of a repeating sequence (e.g., a motif) within a tandem repeat region of the sample genomic sequence 502. In addition, the insert length prediction system 106 predicts or determines a number of nucleobases for the predicted insert length 212 based on the length of the repeating sequence within the tandem repeat region. For example, the insert length prediction system 106 determines a distribution of insert lengths that vary (from an expected value) based on integer multiples of the length of the tandem repeat region.

As further illustrated in FIG. 2, the insert length prediction system 106 performs a read mapping 214. In particular, the insert length prediction system 106 maps the reads of the sample genomic sequence 206 to a reference genome. For example, the insert length prediction system 106 maps the first nucleotide read (“Read 1”) and the second nucleotide read (“Read 2”) of the nucleotide read pair to the reference genome. In some cases, the insert length prediction system 106 maps the nucleotide reads by determining a genomic region (including one or more genomic coordinates or loci) of the reference genome at which the nucleotide reads are likely located. In some cases, the insert length prediction system 106 determines a particular genomic region of the reference genome that maps to the first and second read of the read pair by reflecting or including matching nucleotide bases at each (or a threshold number/percentage) of the genomic coordinates within the genomic region.

Additionally, as shown, the insert length prediction system 106 generates a genotype call 216 (or multiple genotype calls). Particularly, the insert length prediction system 106 generates the genotype call 216 based on the read mapping 214 (which is in turn based on the predicted insert length 212). For instance, the insert length prediction system 106 generates a genotype call to indicate a genotype at a particular genomic coordinate of the sample genomic sequence 206 (e.g., in relation to the reference genome).

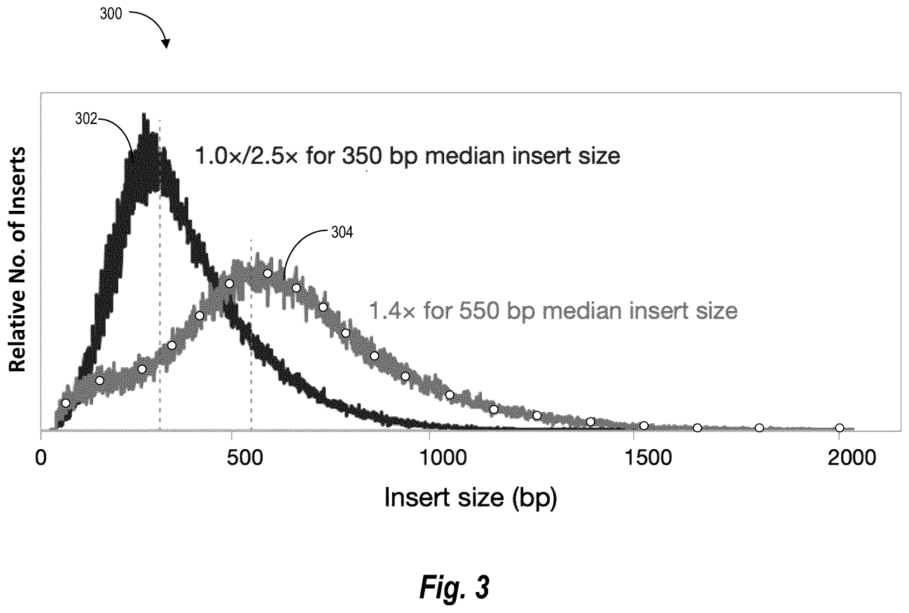

As mentioned above, in certain described embodiments, the insert length prediction system 106 improves the accuracy of predicting insert lengths over existing sequencing systems. Indeed, experimenters have demonstrated the inaccuracy of insert length predictions produced by existing sequencing systems. FIG. 3 illustrates an example graph of distributions of insert lengths generated for two different types of library preparation in accordance with one or more embodiments.

To generate the graph 300, experimenters utilize a prior sequencing system to generate variant calling metrics (e.g., using a variant caller) for determining insert lengths. Experimenters further plot probability distributions of inserts having different insert sizes as determined from the variant calling metrics. Indeed, as illustrated in FIG. 3, the graph 300 portrays a first curve 302 representing predicted insert sizes (in numbers of base pairs or “bp”) for a number of inserts (e.g., based on a first type of library preparation), based on an experiment performed to target a median insert size of 350 base pairs, where the median insert size is indicated by the dashed line intersecting near the peak of the first curve 302. As also illustrated in FIG. 3, the graph 300 portrays a second curve 304 representing predicted insert sizes for inserts based on an experiment performed to target a median insert size of 500 base pairs (e.g., based on a second type of library preparation), as indicated by the dashed line intersecting near the peak of the second curve 304. Indeed, the graph 300 reflects probabilities of inserts having respective insert sizes of 350 or 500 base pairs for the two experiments.

As reflected in the graph 300, neither the first curve 302 nor the second curve 304 exhibit or reflect the shape of a normal (e.g., Gaussian) probability distribution. Indeed, as shown, each of the curves has a very wide range of potential insert lengths with abnormal distributions. Without better metrics and models for determining more accurate insert lengths, existing sequencing systems thus suffer from inaccurate predictions of insert lengths, as demonstrated by the experimenters and as reflected in the graph 300. This is especially true for repeat regions of a genomic sequence where there are many potential ways to map a read pair.

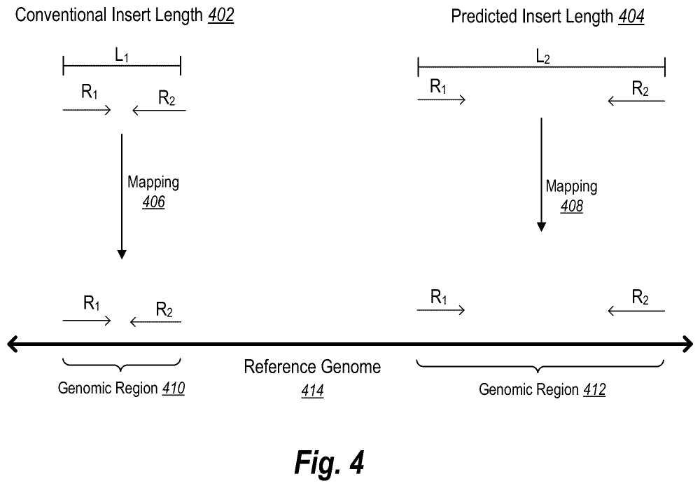

As mentioned above, in certain described embodiments, the insert length prediction system 106 generates more accurate predicted insert lengths than existing sequencing systems. In addition, the insert length prediction system 106 utilizes cluster metrics determined from primary analysis processes to determine insert lengths, which renders the insert length useful for informing secondary analysis processes, such as mapping and genotype calling. Indeed, the insert length prediction system 106 can more accurately map nucleotide reads than existing sequencing systems based on predicted insert lengths. FIG. 4 illustrates an example diagram showing improved mapping in accordance with one or more embodiments.

As illustrated in FIG. 4, diagram compares mapping performance of the insert length prediction system 106 and a conventional system based on respective insert length predictions. While some existing sequencing systems determine insert lengths using variant calling metrics and cannot, therefore, use the insert lengths to performing prior processes such as mapping, the diagram of FIG. 4 pictorially demonstrates a hypothetical scenario where, even if a prior system were able to map paired reads using its predicted insert length, the insert length prediction system 106 nevertheless maps more accurately due to a more accurate insert length prediction.

Indeed, as illustrated in FIG. 4, the insert length prediction system 106 generates a predicted insert length 404 having a different length or size (e.g., a different number of nucleobases represented by “L2”) than a conventional insert length 402 (“L1”) determined via a prior sequencing system. More specifically, the insert length prediction system 106 generates the predicted insert length 404 using the methods and processes described herein to determine or predicted a number of nucleobases within an insert of a fragment or a genomic sequence.

Based on the predicted insert length 404, the insert length prediction system 106 further performs a mapping 408 that exhibits accuracy improvements over the mapping 406 of the prior system. To elaborate, the mapping 406 indicates that the prior system analyzes a reference genome 414 to identify or select a genomic region 410 as corresponding to (or mapping to) the paired nucleotide reads R1 and R2 of the prior system. By contrast, insert length prediction system 106 applies a mapping 408 to identify or select a different genomic region 412 within the reference genome 414. Indeed, the insert length prediction system 106 selects the different genomic region 412 to map the paired nucleotide reads R1 and R2 more accurately, as informed by the predicted insert length 404. Thus, as shown, the insert length prediction system 106 improves mapping accuracy over existing sequencing systems as a result of more accurately determining or generating predicted insert lengths.

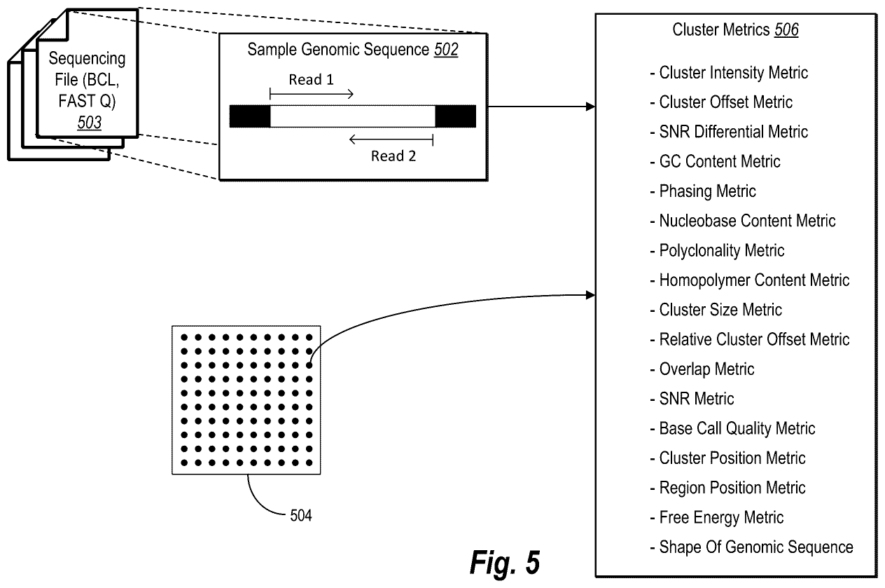

As mentioned above, in certain described embodiments, the insert length prediction system 106 generates cluster metrics as a basis for predicting an insert length. In particular, the insert length prediction system 106 generates or extracts cluster metrics using one or more sequencing runs to analyze a cluster of a flow cell (and/or a particular genomic sequence within the cluster). FIG. 5 illustrates an example diagram for generating cluster metrics in accordance with one or more embodiments.

As illustrated in FIG. 5, the insert length prediction system 106 determines cluster metrics 506 from a sample genomic sequence 502 and/or from a cluster of a nucleotide-sample slide 504. To conserve space and depict clusters, FIG. 5 depicts only a region of the nucleotide-sample slide 504. Indeed, as mentioned above, the insert length prediction system 106 can determine or extract particular metrics or measurements for a cluster or well of the nucleotide-sample slide 504 (e.g., a cluster/well where the sample genomic sequence 502 is synthesized). In addition, the insert length prediction system 106 can generate or extract particular metrics or measurements specific to (one or more reads in a nucleotide read pair of) the sample genomic sequence 502. Indeed, the insert length prediction system 106 can generate a sequencing file 503 (e.g., a BCL file or a FASTQ file) that includes sequencing data for the sample genomic sequence 502 used to generate one or more of the cluster metrics 506.

To generate the sequencing file 503, in some embodiments, the insert length prediction system 106 (via the sequencing device 114) utilizes cluster generation and SBS chemistry to sequence millions or billions of clusters in a flow cell. During SBS chemistry, for each cluster, the sequencing device 114 (or the insert length prediction system 106) stores nucleobase calls for every cycle of sequencing via real-time analysis (RTA) software. The sequencing device 114 (or the insert length prediction system 106) utilizes RTA software to further store base call data in the form of individual base call data files (or BCLs). In some cases, the sequencing device 114 (or the insert length prediction system 106) further converts the BCL files into sequence data (e.g., via BCL to FASTQ conversion). For instance, the sequencing device 114 (or the insert length prediction system 106) generates FASTQ files from nucleotide reads (e.g., Read 1 and Read 2).

In some cases, the insert length prediction system 106 generates sequence data for each cluster that passes an initial quality filter from the sample genomic sequence 502. For example, the insert length prediction system 106 generates entries for each cluster, where each entry includes four lines (or four items of sequence data): i) a sequence identifier with information about the sequencing run and the cluster, ii) nucleobase calls that make up the sequence (e.g., a sequence of A, C, T, G, and/or N calls), iii) a separator (e.g., a “+” sign), and iv) base-call-quality metrics indicating probabilities of correctness for the nucleobase calls (PHRED +33 encoded).

As just mentioned, the insert length prediction system 106 can generate cluster metrics 506 which include metrics that inform the prediction of an insert length. As an example of a cluster metric, the insert length prediction system 106 generates or determines a cluster intensity metric or a signal intensity metric. To elaborate, the insert length prediction system 106 determines a cluster intensity metric that indicates an intensity of a signal emitted from a cluster of oligonucleotides within the nucleotide-sample slide 504. For instance, the cluster intensity metric can indicate an intensity of voltage (or other electromagnetic signals), photon emission, and/or light emission from a cluster. In some cases, the insert length prediction system 106 determines the cluster intensity metric by capturing (e.g., via an imaging device, such as CCD or CMOS, or a light sensor) an intensity of emitted light from the cluster in response to laser stimulation at a particular time during a sequencing run or a sequencing cycle. For instance, the insert length prediction system 106 projects a laser at the nucleotide-sample slide 504 and captures measurements of light emitted or reflected back from (clusters in) the nucleotide-sample slide 504 in one or more light channels. In certain embodiments, the insert length prediction system 106 determines an average emitted light intensity from the cluster over two or more (portions of) cycles and/or at two or more sampling times during a sequencing cycle and/or over two or more captured light channels.

In one or more embodiments, the insert length prediction system 106 determines a cluster intensity metric based on a correlation between the intensity of different wells or clusters of the nucleotide-sample slide 504. More specifically, the insert length prediction system 106 determines a channel estimate for each well/cluster of the nucleotide-sample slide 504 over one or more captured light channels. From the channel estimate, the insert length prediction system 106 can determine a relative intensity (or a normalized intensity) for a cluster in relation to the intensities of light emitted from other clusters of the nucleotide-sample slide 504. Indeed, the insert length prediction system 106 can compare emitted light intensities from different clusters/wells of the nucleotide-sample slide 504 to determine relative cluster intensity metrics.

In certain embodiments, the insert length prediction system 106 determines multiple cluster intensity metrics, one for each color (e.g., laser wavelength) or channel of light associated with a sequencing run or cycle. For example, the insert length prediction system 106 utilizes a multi-channel laser (or multi-channel image capturing) to stimulate clusters of the nucleotide-sample slide 504. The insert length prediction system 106 can thus determine a cluster intensity for each channel or each color of the laser or the captured image. In some cases, the insert length prediction system 106 determines a cluster intensity metric by averaging the channel-specific intensities for a subset of sequencing cycles (e.g., for cycle 15 at the middle of the read and/or cycle 100 at the end of the read). Thus, the insert length prediction system 106 can generate channel-specific averages for different (subsets of) cycles (e.g., every fifth cycle) to determine a cluster intensity metric.

In some cases, the cluster intensity metric is a raw intensity (e.g., an intensity of light directly read out from sensors), a partially corrected intensity (e.g., an intensity corrected by a system or a computer that corrects or adjusts light intensity read from clusters, such as an equalizer), or a fully corrected intensity. In these or other cases, the insert length prediction system 106 determines a cluster intensity metric in the form of a Y-cluster scaling coefficient to normalize cluster intensities relative to one another for the nucleotide-sample slide 504. Thus, the insert length prediction system 106 can determine a Y-cluster scaling coefficient based on raw intensity values, partially corrected intensity values, or fully corrected intensity values. Indeed, the insert length prediction system 106 can determine a Y-cluster scaling coefficient based on any combination of factors used to determine a cluster intensity metric described herein.

As another of the cluster metrics 506, the insert length prediction system 106 determines a cluster gain metric. In particular, the insert length prediction system 106 determines a cluster gain metric that indicates a difference between signals (e.g., light, voltage, or photons) emitted in different states of a cluster. For example, clusters of the nucleotide-sample slide 504 can have emitting (e.g., luminescent) states and non-emitting (e.g., non-luminescent) states. Indeed, the insert length prediction system 106 can project a laser at the nucleotide-sample slide 504 to determine a light intensity emitted from a cluster in a luminescent state. In addition, the insert length prediction system 106 can determine a light intensity emitted from the cluster in a non-luminescent state when no laser is projected at the nucleotide-sample slide 504. Thus, to generate a cluster gain metric, the insert length prediction system 106 can compare the light intensity from the first (luminescent state) state and the second (non-luminescent) state to determine a difference in the respective emitted light intensities.

As another of the cluster metrics 506, the insert length prediction system 106 determines a cluster offset metric. In particular, the insert length prediction system 106 determines a cluster offset metric that indicates an estimate or a measure of background noise (e.g., signal noise from light, voltage, or photons) for a cluster of oligonucleotides. In some embodiments, the insert length prediction system 106 determines a cluster offset metric by determining readings of the nucleotide-sample slide 504 in a state where wells do not include clusters of oligonucleotides (e.g., where the wells have no tags). For example, clusters of the nucleotide-sample slide 504 exhibit a measure of signal intensity (e.g., voltage, photon, or light) even when not stimulated by a laser, electricity, or some other trigger. Thus, for the cluster offset metric, the insert length prediction system 106 can determine or measure a signal intensity during a non-illuminated state for an oligonucleotide cluster of the nucleotide-sample slide 504. In some cases, the insert length prediction system 106 determines the cluster offset metric at the end of one or both reads because background noise builds up over reads and has likely accumulated the most by the end of the read.

Similar to the cluster intensity metric, the insert length prediction system 106 can determine the cluster offset metric (and/or the cluster gain metric) for different colors or channels and/or for different (subsets of) sequencing cycles. For example, the insert length prediction system 106 can determine an average cluster offset for cycle 15 and/or cycle for different reads and/or for different color channels.

As yet another of the cluster metrics 506, the insert length prediction system 106 can determine a signal-to-noise ratio (“SNR”) differential metric. More specifically, the insert length prediction system 106 determines an SNR differential metric that indicates a difference between an SNR for a first nucleotide read (“Read 1”) and an SNR for a second nucleotide read (“Read 2”) of a nucleotide read pair. Indeed, the insert length prediction system 106 analyzes the sample genomic sequence 502 (or the sequencing file 503) to determine a first SNR for the first nucleotide read and a second SNR for the second nucleotide read. In addition, the insert length prediction system 106 compares the first SNR and the second SNR to determine the SNR differential metric (e.g., as a ratio of SNRs). In some cases, the insert length prediction system 106 determines the SNR differential metric for different (subsets of) cycles and/or for different channels/colors used in a sequencing run.

As a further example of the cluster metrics 506, the insert length prediction system 106 generates or determines a guanine-cytosine (“GC”) content metric. For example, the insert length prediction system 106 determines a GC content metric that indicates an amount of sequenced nucleotide bases within a nucleotide read pair that include dinucleotide repeats of guanine and/or cytosine. Indeed, the insert length prediction system 106 determines a GC content metric by analyzing the first nucleotide read (Read 1) and/or the second nucleotide read (Read 2) to identify dinucleotide repeats of a guanine base and/or a cytosine base. Accordingly, the insert length prediction system 106 generates a numerical representation for the GC content metric to indicate an amount, a percentage, a ratio, or a total count of GC content within one or more of the first read or the second read. In some cases, the insert length prediction system 106 determines melting temperatures for GC content and/or a sample genomic sequence as part of the cluster metrics 506.

As further illustrated in FIG. 5, the insert length prediction system 106 determines a phasing metric as part of the cluster metrics 506. More particularly, the insert length prediction system 106 determines a phasing metric that indicates phasing or pre-phasing of oligonucleotides within the oligonucleotide cluster from the nucleotide-sample slide 504. As used herein, the term “phasing” refers to an instance of (or rate at which) labeled nucleotide bases are incorporated behind a particular sequencing cycle. For example, the insert length prediction system 106 determines phasing by identifying or detecting an instance of (or rate at which) labeled nucleotide bases within a cluster are asynchronously incorporated behind other labeled nucleotide bases within the cluster for a particular sequencing cycle. In particular, during SBS, each DNA strand in a cluster extends incorporation by one nucleotide base per cycle. One or more oligonucleotide strands within the cluster may become out of phase with the current cycle. Phasing occurs when nucleotide bases for one or more oligonucleotides within a cluster fall behind one or more cycles of incorporation. For example, a nucleotide sequence from a first location to a third location may be CTA. In this example, the C nucleotide should be incorporated in a first cycle, T in the second cycle, and A in the third cycle. When phasing occurs during the second sequencing cycle, one or more labeled C nucleotides are incorporated instead of a T nucleotide.

Relatedly, as used herein, the term “pre-phasing” refers to an instance of (or rate at which) one or more nucleotide bases are incorporated ahead of a particular cycle. Pre-phasing includes an instance of (or rate at which) labeled nucleotide bases within a cluster are asynchronously incorporated ahead other labeled nucleotide bases within a cluster for a particular sequencing cycle. To illustrate, when pre-phasing occurs during the second sequencing cycle in the example above, one or more labeled A nucleotides are incorporated instead of a T nucleotide. Thus, the insert length prediction system 106 can generate a phasing metric by determining phasing and/or pre-phasing of oligonucleotides within the cluster from the nucleotide-sample slide 504.

In addition, the insert length prediction system 106 can determine a nucleobase content metric as part of the cluster metrics 506. In particular, the insert length prediction system 106 determines a nucleobase content metric that indicates amounts of sequenced nucleotide bases within the first nucleotide read (Read 1) or the second nucleotide read (Read 2) that are adenine, cytosine, guanine, or thymine bases. For example, the insert length prediction system 106 determines ratios, percentages, or total counts of each type nucleobase within Read 1 and Read 2. In some cases, the insert length prediction system 106 determines a nucleobase content metric that indicates nucleobase content for both reads together, or that indicates separate nucleobase content for each of Read 1 and Read 2 independently. In certain embodiments, the insert length prediction system 106 generates a nucleobase content metric that includes different metrics or numbers for the content (e.g., a ratio or a percentage) of each the read(s) made up by each base type: adenine, cytosine, guanine, and thymine.

As further illustrated in FIG. 5, the insert length prediction system 106 generates or determines a polyclonality metric as part of the cluster metrics 506. More specifically, the insert length prediction system 106 determines a polyclonality metric that indicates a probability that a cluster of oligonucleotides includes oligonucleotides from two or more genomic samples. For example, the insert length prediction system 106 analyzes the cluster from the nucleotide-sample slide 504 where the sample genomic sequence 502 is synthesized to determine a probability that the cluster includes DNA (or oligonucleotides) from two or more genomic samples (e.g., from more than one organism).

Additionally, as shown in FIG. 5, the insert length prediction system 106 determines a homopolymer content metric as part of the cluster metrics 506. In particular, the insert length prediction system 106 determines a homopolymer content metric that indicates an amount of homopolymer content within a cluster of oligonucleotides. For example, the insert length prediction system 106 analyzes the cluster from which the sample genomic sequence 502 is extracted to determine homopolymer content of the cluster. Specifically, the insert length prediction system 106 identifies or detects homopolymer content by detecting copies of a single repeating nucleobase (or multiple repeating nucleobase) within the cluster (or within the sample genomic sequence 502). For instance, the insert length prediction system 106 determines that the cluster includes repeating sequences of A, C, T, and/or G in one or more locations, where a repeating sequence includes at least a threshold number of consecutive occurrences of the same nucleobase. Accordingly, the insert length prediction system 106 can generate the homopolymer content metric to numerically represent a ratio, a percentage, or a count of (bases within) homopolymers (e.g., sequences of repeating bases) for all nucleobase types collectively, or separately for each respective nucleobase type.

As further illustrated in FIG. 5, the insert length prediction system 106 determines or generates a cluster size metric as part of the cluster metrics 506. To elaborate, the insert length prediction system 106 determines a cluster size metric by determining a size of a cluster of oligonucleotides that includes the sample genomic sequence 502. Specifically, the insert length prediction system 106 determines a cluster size by measuring the cluster size within a sequencing image taken during a sequencing run. For instance, the insert length prediction system 106 can measure one or more characteristics of light captured from the cluster within a sequencing image, such as an area, a color, and/or an intensity of the light to determine the cluster size. In some cases, the insert length prediction system 106 determines an average size of the cluster across sequencing images captured over a certain number of cycles within a sequencing run.

In addition, the insert length prediction system 106 determines a relative cluster offset metric as part of the cluster metrics 506. More specifically, the insert length prediction system 106 determines a relative cluster offset by determining a difference in signal (e.g., voltage, photon, or light) intensity emitted from a cluster of oligonucleotides compared to an average signal intensity for a sequencing well in which the cluster of oligonucleotides is located. For instance, during a sequencing run, the insert length prediction system 106 can analyze the light intensity emitted from a cluster located with a particular sequencing well of the nucleotide-sample slide 504. The insert length prediction system 106 can further compare the emitted intensity from the cluster with an emitted intensity from a well center. For instance, the insert length prediction system 106 measures or determines a light intensity emitted from a well center. In some cases, the insert length prediction system 106 determines the light intensity for the well center by determining an average light intensity for the well over a certain number of sequencing runs (or cycles). The insert length prediction system 106 further compares the average intensity for the well with that of the cluster of the sample genomic sequence 502 to generate a relative cluster offset metric.

As also illustrated in FIG. 5, the insert length prediction system 106 determines or generates an overlap metric as part of the cluster metrics 506. In particular, the insert length prediction system 106 determines an overlap metric by determining a number of overlapping nucleobases in a shared sequence between the first nucleotide read (Read 1) and the second nucleotide read (Read 2). For example, the insert length prediction system 106 analyzes the paired nucleotide read of the sample genomic sequence 502 to determine a number of overlapping bases between Read 1 and Read 2. Indeed, in some cases, an insert size of a genomic sequence is less than the length of two paired reads end-to-end, and the insert length prediction system 106 can determine an overlap metric by determining a number of nucleobases within the overlapping portion of the sequence.

Additionally, as shown, the insert length prediction system 106 determines an SNR metric as part of the cluster metrics 506. For example, the insert length prediction system 106 determines an SNR metric by determining a signal-to-noise ratio at one or more of any portion (e.g., an end) of the first nucleotide read or any portion (e.g., an end) of the second nucleotide read. Indeed, the insert length prediction system 106 determines an SNR at one or more locations of the sample genomic sequence 502. For instance, the insert length prediction system 106 determines an SNR at the initial location and/or the end location of Read 1 and/or at the initial location and/or the end location of Read 2. Thus, the insert length prediction system 106 can generate an SNR metric by combining SNR measurements at one or more locations of one or more reads within a nucleotide read pair.

As further shown in FIG. 5, the insert length prediction system 106 can determine a base call quality metric. In particular, the insert length prediction system 106 determines a base call quality metric by determining or measuring an accuracy or confidence with which a base call is determined based on various sequencing metrics (e.g., chastity). For example, the insert length prediction system 106 determines a Q score or a QUAL score for a base call within the sample genomic sequence 502 (e.g., as part of Read 1 or Read 2). Indeed, the insert length prediction system 106 determines a base call quality metric by measuring an accuracy of a nucleobase call. For instance, the insert length prediction system 106 determines a base call quality metric by determining a value indicating a likelihood that one or more predicted nucleobase calls for a genomic coordinate of the sample genomic sequence 502 contain errors. For example, in certain implementations, a base call quality metric can comprise a Q score (e.g., a PHil's Read EDitor (PHRED) quality score) predicting the error probability of any given nucleobase call. To illustrate, a quality metric (or Q score) may indicate that a probability of an incorrect nucleobase call at a genomic coordinate is equal to 1 in 100 for a Q20 score, 1 in 1,000 for a Q30 score, 1 in 10,000 for a Q40 score, etc.

As further illustrated in FIG. 5, the insert length prediction system 106 determines a cluster position metric as part of the cluster metrics 506. In particular, the insert length prediction system 106 determines a cluster position metric by determining a position of the cluster of oligonucleotides within a region of a nucleotide-sample slide (e.g., within the nucleotide-sample slide 504). In some cases, the insert length prediction system 106 determines the cluster position as a coordinate location or a designated well location within the nucleotide-sample slide 504.

Additionally, as shown in FIG. 5, the insert length prediction system 106 determines a region position metric as part of the cluster metrics 506. To elaborate, the insert length prediction system 106 determines a region position metric by determining a position of the region within the nucleotide-sample slide (e.g., within the nucleotide-sample slide 504). For example, the insert length prediction system 106 determines a region within the nucleotide-sample slide 504 where the cluster of the sample genomic sequence 502 is located. Indeed, in many cases, flow cells have different regions of wells arranged in a grid or some other pattern, each with its own label or designation. Thus, the insert length prediction system 106 can determine a region of the nucleotide-sample slide 504 where the cluster of the sample genomic sequence 502 was synthesized and/or extracted.

Further, as shown in FIG. 5, the insert length prediction system 106 determines a free energy metric as part of the cluster metrics 506. More particularly, the insert length prediction system 106 determines an amount of free energy associated with the sample genomic sequence 502 (or a molecule including the sample genomic sequence 502). In some cases, the insert length prediction system 106 determines the free energy by determining a minimum amount of energy required to fold a molecule, such as a molecule containing or made up of the sample genomic sequence 502. In some embodiments, the free energy indicates an amount of energy for folding the sample genomic sequence 502 one or more times and/or to fold the sample genomic sequence 502 into a particular shape or state. Specifically, the free energy indicates a measure of energy for folding a molecule/genomic sequence into a lowest energy state.

In one or more embodiments, the insert length prediction system 106 determines a shape of the sample genomic sequence 502 as part of the cluster metrics 506. More specifically, the insert length prediction system 106 determines a shape of the sample genomic sequence 502 within a chromosome. Indeed, genomic sequences can have a variety of shapes within chromosomes, and the makeup of the genomic sequences influences or impacts such shapes. For instance, the number of nucleobases within a genomic sequence (e.g., its insert length) can impact the shape of the sequence within a chromosome. Accordingly, the insert length prediction system 106 can determine a shape of the sample genomic sequence 502 as an addition cluster metric for determining insert length.

In certain embodiments, the insert length prediction system 106 determines a Phi-X metric. For example, the insert length prediction system 106 determines metrics associated with the Phi-X bacteriophage and compares the Phi-X metrics with metrics associated with the sample genomic sequence 502. Indeed, the insert length prediction system 106 can use the known data about Phi-X as a ground truth for comparing against predicted metrics (e.g., the cluster metrics 506) to inform the accuracy of the predictions. In some cases, the insert length prediction system 106 can use the Phi-X data to train a model, as described in further detail below.

In some embodiments, the insert length prediction system 106 weights the cluster metrics 506 as part of predicting an insert length. For example, the insert length prediction system 106 determines the cluster metrics 506 and applies different weights to each respective cluster weight, depending on its impact or influence on an insert length prediction. In some cases, the insert length prediction system 106 weights the cluster intensity metric most heavily as the intensity of often the most indicative or most influential factor for predicting insert lengths.

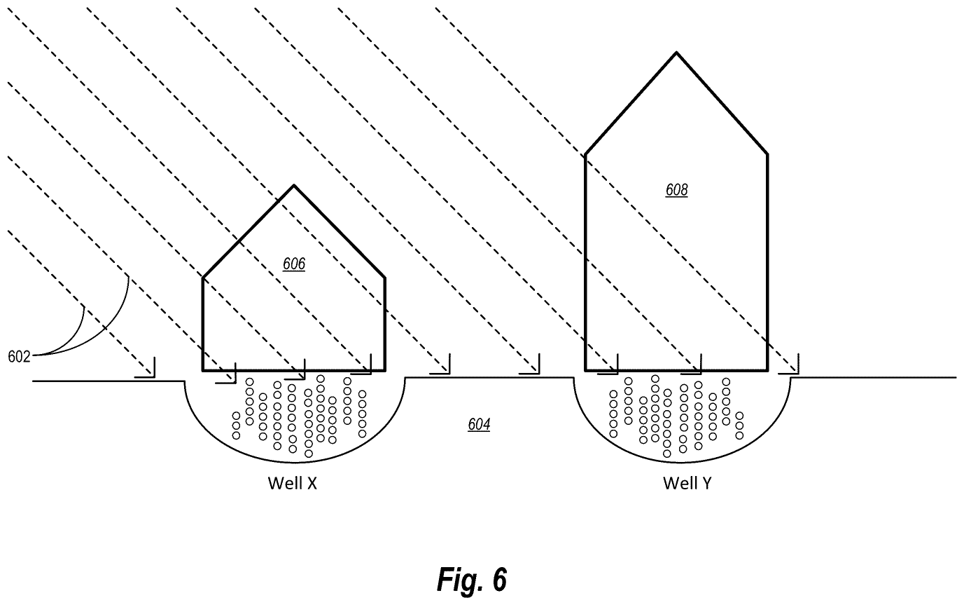

As mentioned above, in certain described embodiments, the insert length prediction system 106 generates or determines a cluster intensity. Indeed, in some cases, the insert length prediction system 106 determines a cluster intensity as a measure of light intensity emitted from a cluster or a well within a flow cell. FIG. 6 illustrates an example closeup side view of a flow cell for determining a cluster intensity in accordance with one or more embodiments.

As illustrated in FIG. 6, the insert length prediction system 106 projects a laser 602 (or some other light) onto a flow cell 604 as part of a sequencing run within an SBS process. The insert length prediction system 106 thus stimulates clusters of oligonucleotides within respective wells throughout the flow cell 604, including Well X and Well Y. Indeed, as shown, the insert length prediction system 106 projects the laser 602 onto the cluster of Well X and the cluster of Well Y. In response to stimulation from the laser 602, the cluster of Well X emits light 606 represented by a vertical arrow. In addition, the cluster of Well Y emits light 608 in response to stimulation from the laser 602. As shown, the intensity of the emitted light from the clusters is represented by the height of the respective arrows representing the light 606 and the light 608. Comparing the light 606 and the light 608, the insert length prediction system 106 determines that the cluster of Well Y has a greater cluster intensity metric than the cluster of Well X.

In some embodiments, the insert length prediction system 106 projects the laser 602 at the flow cell 604 using one or more light channels. In these or other embodiments, the insert length prediction system 106 projects the laser 602 at certain intervals over one or more cycles and/or over one or more sequencing runs. Thus, the insert length prediction system 106 can determine various forms of a cluster intensity metric. For instance, the insert length prediction system 106 determines an average intensity of light emitted over multiple channels captured by an imaging device (e.g., CCD or CMOS) or a light sensor (and/or for multiple channels projected by the laser 602). In addition, the insert length prediction system 106 can capture light intensity measurements at particular intervals (or sample times) of a cycle to determine an average intensity of light over multiple cycles. Similarly, the insert length prediction system 106 can capture light intensity measurements at particular intervals (or sample times) of a sequencing run to determine an average intensity of light over multiple runs. The insert length prediction system 106 can thus generate a cluster intensity metric using a combination of any of the above techniques.

While FIG. 6 illustrates Well X and Well Y for a patterned flow cell, the insert length prediction system 106 can perform the processes and functions described herein on non-patterned flow cells or on other nucleotide-sample slides as well. For example, the insert length prediction system 106 can measure and determine an average intensity of light emitted by clusters on a non-patterned nucleotide-sample slide over multiple cycles.

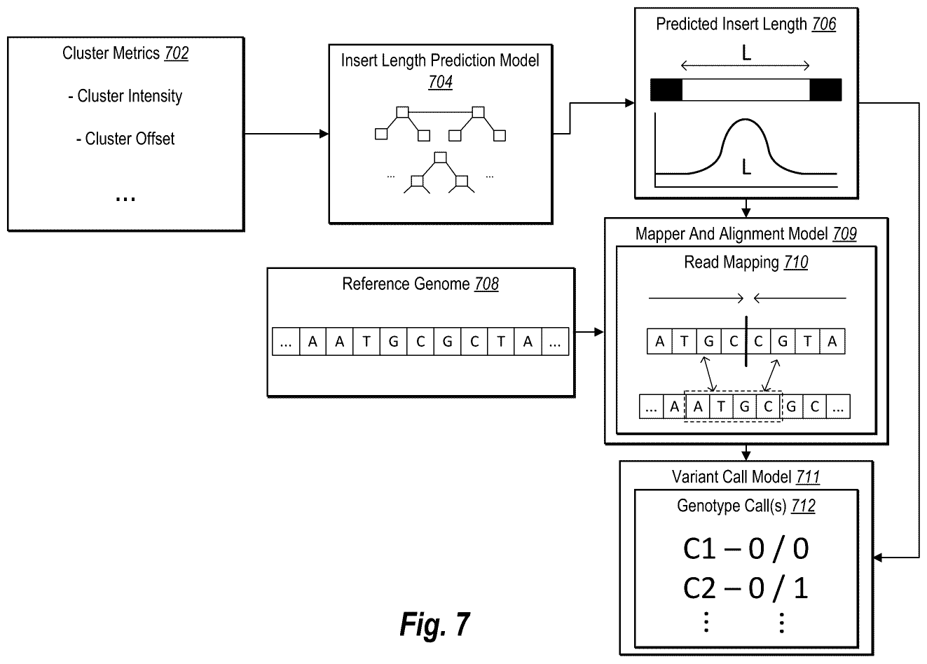

As mentioned above, in certain described embodiments, the insert length prediction system 106 maps nucleotide reads and generates genotype calls based on an insert length. In particular, the insert length prediction system 106 generates a predicted insert length which informs, and improves the accuracy of, mapping and genotype calling. FIG. 7 illustrates an example diagram for mapping and genotype calling based on a predicted insert length in accordance with one or more embodiments.

As illustrated in FIG. 7, the insert length prediction system 106 determines or generates cluster metrics 702. More specifically, the insert length prediction system 106 generates the cluster metrics 702 according to the description above. In addition, the insert length prediction system 106 inputs the cluster metrics 702 into the insert length prediction model 704. Indeed, the insert length prediction system 106 utilizes the insert length prediction model 704 to generate a predicted insert length 706 from the cluster metrics 702. As indicated above, the insert length prediction model 704 can take the form of an ensemble of gradient boosted trees trained on a logistic regression to process the cluster metrics 702 and to converge on a predicted insert length 706 for a sample genomic sequence.

As further illustrated in FIG. 7, the insert length prediction system 106 performs a read mapping 710. To elaborate, the insert length prediction system 106 maps reads of a nucleotide read pair using a mapper and alignment model 709 informed by the predicted insert length 706. For instance, the mapper and alignment model 709 maps and aligns nucleotide reads in relation to a reference genome 708 based on the predicted insert length 706 corresponding to the nucleotide reads of a read pair. Indeed, the mapper and alignment model 709 aligns nucleobases within a read for comparison with corresponding nucleobases within the reference genome 708. In addition, the insert length prediction system 106 identifies a set of candidate genomic regions within the reference genome 708. The insert length prediction system 106 further selects, from among the set of candidate genomic regions, the genomic region for mapping the first nucleotide read and the second nucleotide read based on the predicted insert length. Thus, based on comparing reference bases at candidate genomic regions, the insert length prediction system 106 can identify a genomic region of the reference genome 708 with coordinates that map to the bases of one or more nucleotide reads.

To elaborate, as shown, the insert length prediction system 106 performs a read mapping 710 by mapping a first read of a read pair and a second read of the read pair to a particular genomic region of the reference genome 708. Indeed, using the mapper and alignment model 709, the insert length prediction system 106 can determine nucleobase similarities between nucleobases of the reads and nucleobases in various genomic regions of the reference genome 708. For example, the insert length prediction system 106 can determine a nucleobase similarity in the form of a probability of a particular genomic coordinate within the reference genome 708 reflecting or including the same nucleobase at the corresponding genomic coordinate of a nucleotide read. Based on the nucleobase similarities of various coordinates within regions, the insert length prediction system 106 can further determine probabilities of the first read and second read corresponding to different genomic regions of the reference genome 708 (e.g., based on whether the genomic coordinates in the regions include the same nucleobases as indicated by the reads). Accordingly, the mapper and alignment model 709 generates mapping quality metrics (e.g., MAPQ scores) for different genomic regions of the reference genome 708, where higher mapping quality metrics indicate higher probabilities of corresponding to (e.g., matching) a nucleotide read.

The insert length prediction system 106 can further select a genomic region with at least a threshold mapping quality (e.g., MAPQ). Indeed, as part of the read mapping 710, the insert length prediction system 106 can compare mapping quality metrics for different genomic coordinates or genomic regions to identify a region to which a nucleotide read maps. As shown, the insert length prediction system 106 identifies a genomic region of the reference genome 708 for mapping the first nucleotide read and the second nucleotide read (each indicating bases of “ATGC”). Indeed, the selected genomic region is indicated by the dashed box within the read mapping 710.

In one or more embodiments, as part of the read mapping 710, the insert length prediction system 106 maps a read pair to a genomic region that exhibits or includes particular nucleobase patterns or traits. For instance, the insert length prediction system 106 maps a first nucleotide read and/or a second nucleotide read of a read pair to a genomic region of the reference genome 708 that includes one or more of a structural variant, a variable number tandem repeat (“VNTR”), a short tandem repeat (“STR”), a segmental duplication, a long interspersed nucleotide element (“LINE”), or a short interspersed nucleotide element (“SINE”).

As just mentioned, the insert length prediction system 106 can map one or more reads of a nucleotide read pair to a genomic region such that the read(s) exhibits or includes a variant in relation to the reference genome. As used herein, the term “variant” refers to a nucleobase or multiple nucleobases that do not align with, differs from, or varies from a corresponding nucleobase (or nucleobases) in a reference sequence or a reference genome (e.g., the reference genome 708). For example, a variant includes a single nucleotide polymorphism (“SNP”), an indel, or a structural variant that indicates nucleobases in a sample genomic sequence that differ from nucleobases in corresponding genomic coordinates of a reference sequence. Along these lines, a “variant call” refers to a prediction of whether a variant exists at a genomic coordinate of a genomic sequence (e.g., as compared to a nucleobase at the corresponding coordinate within a reference genome).

In some embodiments, the insert length prediction system 106 maps one or more nucleotides reads of a read pair to a particular genomic region of the reference genome 708 such that, when compared to the reference genome 708, the read(s) exhibit or include a structural variant. As used herein, the term “structural variant” refers to a particular type of variant or variation (e.g., deletion, insertion, translocation, inversion) in a structure of an organism's chromosome or a variation to the nucleotide sequences of the organism's chromosome. In some cases, a structural variant includes a variation to a threshold number of base pairs within an organism's chromosome. Accordingly, in certain implementations, a structural variant includes an insertion or a deletion exceeding a threshold number of base pairs, a duplication exceeding a threshold number of base pairs, an inversion, a translocation, or a copy number variation (“CNV”).

As mentioned above, in some embodiments, the insert length prediction system 106 maps one or more reads of a read pair to a genomic region of the reference genome 708 that includes a variable number tandem repeat or VNTR. In some cases, a VNTR can comprise a location in a genome where a variable-number (but relatively short) nucleotide sequence (e.g., 20-100 base pairs) is organized as a tandem repeat. For example, a VNTR can comprise a sequence made up of six repeating AGTCGGTAAG sequences or various other numbers of repeating subsequences. VNTRs may cause errors in SBS by causing polymerase slippage leading to downstream phasing and pre-phasing. Other examples of VNTRs include minisatellite sequences and microsatellite sequences. Minisatellite sequences refer to tracts of repetitive DNA in which certain DNA motifs (ranging in length from 10-60 base pairs) are typically repeated 5-50 times. Microsatellite sequences are tracts of repetitive DNA in which certain DNA motifs (ranging in length from one to six or more base pairs) are typically repeated 5-50 times.

As mentioned above, in some embodiments, the insert length prediction system 106 maps one or more reads of a read pair to a genomic region of the reference genome 708 that includes a short tandem repeat or STR. An STR can comprise a location in a genome where a short nucleotide sequence (e.g., 2-7 base pairs) is organized as a tandem repeat. For example, an STR can vary in length but is generally shorter than a variable number tandem repeat (e.g., includes fewer base pairs in the repeating segments).

As mentioned above, in some embodiments, the insert length prediction system 106 maps one or more reads of a read pair to a genomic region of the reference genome 708 that includes a segmental duplication or SD. A segmental duplication can comprise a region or a location of a genome that includes low-copy repeats. For example, a segmental duplication can comprise a block or segment of nucleobases within a range (e.g., from 1 to 400 kb in length) that occur at more than one site within a genome. In some cases, a segmental duplication includes a nucleobase segment with a high level (e.g., greater than 90%) of sequence identity with another segment of the genome.

As mentioned above, in some embodiments, the insert length prediction system 106 maps one or more reads of a read pair to a genomic region of the reference genome 708 that includes a long interspersed nucleotide element or LINE. A LINE can comprise a location or a region of a genome that includes a group or a sequence (e.g., in a range from 4 kb to 7 kb long) of retrotransposons (e.g., DNA elements that amplify themselves throughout eukaryotic genomes) that are not long tandem repeats (“LTRs”). For example, a LINE can include retro-transposable elements and can belong to one of five main groups: L1, RTE, R2, I, and Jockey.

As mentioned above, in some embodiments, the insert length prediction system 106 maps one or more reads of a read pair to a genomic region of the reference genome 708 that includes a short interspersed nucleotide element or SINE. A SINE can comprise a location or a region of a genome that includes non-autonomous, non-coding transposable elements that with a particular range (e.g., 100 to 700 base pairs). A SINE can include retrotransposons, where the internal regions are highly conserved, suggesting positive pressure to preserve structure and function. In some cases, a SINE is lineage-specific, making it a useful marker for divergent evolution between species. In addition to its usefulness in certain types of human disease implication, copy number variations and mutations in a SINE sequence make it possible to construct phylogenies based on differences in SINEs between species.

As further illustrated in FIG. 7, the insert length prediction system 106 generates one or more genotype call(s) 712 based on the read mapping 710 (which is based on the predicted insert length 706). Indeed, the insert length prediction system 106 can generate the genotype call(s) 712 using a variant call model 711 (e.g., an ILLUMINA DRAGEN variant caller). For instance, the insert length prediction system 106 can utilize the variant call model 711 in the form of a probabilistic model to process or analyze nucleotide reads of a sample genomic sequence, including nucleotide base calls and associated metrics (e.g., mapping data and insert length). Accordingly, in some cases, the variant call model 711 may refer to a Bayesian probability model that generates genotype calls and variant calls based on nucleotide reads of a genomic sequence. For instance, the variant call model 711 can process or analyze sequencing metrics corresponding to read pileups (e.g., multiple nucleotide reads corresponding to a single genomic coordinate), including mapping quality, base quality, and various hypotheses including foreign reads, missing reads, joint detection, and more. The variant call model 711 may likewise include multiple components, including, but not limited to, different software applications or components for mapping and aligning, sorting, duplicate marking, computing read pileup depths, genotype calling, and variant calling.

In one or more embodiments, the insert length prediction system 106 utilizes the variant call model 711 to determine a genotype call for a genomic coordinate within a structural variant, a VNTR, an STR, a segmental duplication, a LINE, or a SINE (e.g., corresponding to the genomic region of the reference genome 708 where nucleotide reads are mapped). As shown, the insert length prediction system 106 generates a first genotype of 0/0 (e.g., homozygous-reference or “hom-ref”) for genomic coordinate C1. In addition, the insert length prediction system 106 generates a second genotype of 0/1 (e.g., heterozygous) for genomic coordinate C2.

In some embodiments, the insert length prediction system 106 utilizes the variant call model 711 to generate the genotype call(s) 712 in the form of indels. For instance, the variant call model 711 processes data from the mapper and alignment model 709 and/or processes the predicted insert length 706 to determine or predict that an indel exists within a sample genomic sequence. In some implementations, the insert length prediction system 106 determines that an indel exists by comparing the predicted insert length 706 with an expected insert length for the sample genomic sequence. For example, if the predicted insert length 706 is shorter (e.g., by a threshold number of nucleobases) than the expected length, then the insert length prediction system 106 can determine that the sample genomic sequence includes a deletion.

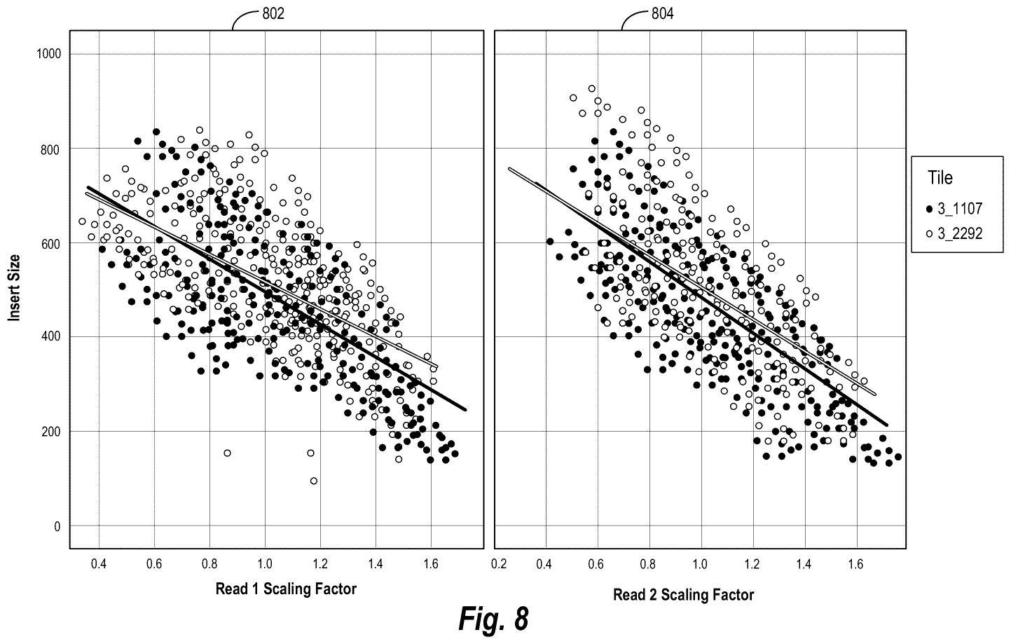

As mentioned above, in certain embodiments, the insert length prediction system 106 utilizes cluster intensity as an indicative or informative measure for predicting insert length. Indeed, experimenters have demonstrated the relationship between insert length and cluster intensity. FIG. 8 illustrates an example visualization of the relationship between insert length and cluster intensity in accordance with one or more embodiments.

As illustrated in FIG. 8, a graph 802 depicts experimental results for a first nucleotide read of a read pair, and the graph 804 depicts experimental results for a second nucleotide read of the read pair. Indeed, experimenters tested the correlation between insert size (on the Y-axis of each graph) and a scaling factor (on the X-axis of each graph), which indicates or represents a cluster intensity metric. For instance, graph 802 and graph 804 each include plot points for two different tiles (or regions) of a nucleotide-sample slide (e.g., flow cell), which each include a number of wells for synthesizing oligonucleotide clusters. Specifically, the tile identifications shown in the graphs are tile 3_1107 (in closed black dots) and tile 32292 (in empty while circles).

As shown in graph 802 and in graph 804, the trend lines indicate a strong (and inverse) relationship between insert size and cluster intensity. Indeed, as cluster intensity increases along the X-axis, the insert size decreases for almost all of the plot points, with a few outliers. Thus, if the insert length prediction system 106 determines a high cluster intensity for a cluster, the insert size is likely shorter for a genomic sequence from the cluster. Probabilistically, the experiments of FIG. 8 demonstrate and illustrate a strong correlation between cluster intensity and insert length.

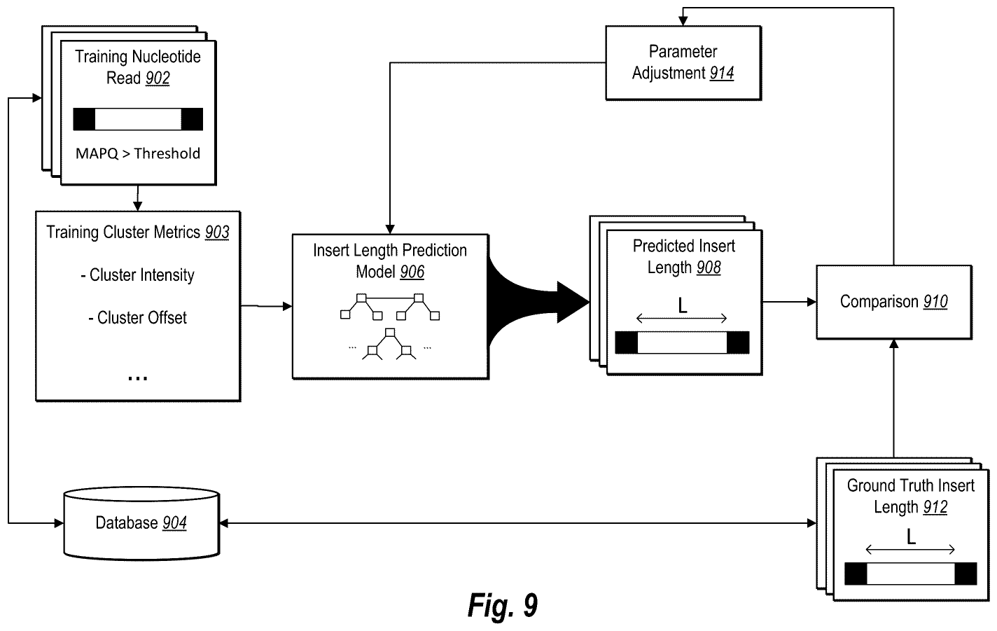

As mentioned above, in one or more embodiments, the insert length prediction system 106 trains an insert length prediction model to accurately predict insert lengths of genomic sequences. In particular, the insert length prediction system 106 selects and utilizes training data in the form of nucleotide reads with mapping qualities that satisfy a threshold as training samples for the model. FIG. 9 illustrates an example training diagram for learning parameters of an insert length prediction model in accordance with one or more embodiments.

As illustrated in FIG. 9, the insert length prediction system 106 trains or tunes an insert length prediction model. In particular, the insert length prediction system 106 trains an insert length prediction model by learning model parameters from training data, such as sample nucleotide reads of a genomic sequence. FIG. 9 illustrates an example training process for learning parameters of an insert length prediction model in accordance with one or more embodiments.

As illustrated in FIG. 9, the insert length prediction system 106 identifies or determines a training nucleotide read 902 as training data for an insert length prediction model 906. For instance, the insert length prediction system 106 identifies the training nucleotide read 902 from a database 904 that stores a plurality of nucleotide reads (e.g., in the form of sequencing data files, such as BCLs and/or FASTQ files and/or in the form of variant call files or VCFs) for one or more genomic sequences. More specifically, the insert length prediction system 106 analyzes a plurality of nucleotide reads for one or more sample genomic sequences to identify and select the training nucleotide read 902 as a nucleotide read having a mapping quality metric (e.g., a MAPQ score) that satisfies a threshold mapping quality. For example, the insert length prediction system 106 determines mapping quality metrics for a number of nucleotide reads (e.g., within read pairs) for one or more genomic sequences. The insert length prediction system 106 further compares the mapping quality metrics with a mapping quality threshold to identify those nucleotide reads with at least a threshold mapping quality as training nucleotide reads. Indeed, in some cases, those reads with higher mapping qualities are more reliable to use as training data because the system is more certain of their correct mapping to a reference genome.

To identify such training nucleotide reads, in some cases, the insert length prediction system 106 identifies candidate nucleotide reads from genomic regions of a genome that are known to be less likely to have structural variants and exhibit at least the threshold mapping quality. For instance, the insert length prediction system 106 identifies candidate nucleotide reads that map to one or more of centromeres, telomers, housekeeping genes (e.g., ACTB, GAPDH, HPRT, ARBP, SDHA, UBC, PGK1, and YWHAZ), enhancer or promoter regions, ribosomal RNA genes (rRNA), transfer RNA genes (tRNA), or microRNAs (miRNA).

As further illustrated in FIG. 9, the insert length prediction system 106 inserts training cluster metrics 903 (extracted from the training nucleotide read 902) into the insert length prediction model 906. Indeed, the insert length prediction system 106 generates or extracts the training cluster metrics 903 from the training nucleotide read 902, as described above, and/or other training nucleotide reads corresponding to different genomic coordinates. As described, the training cluster metrics 903 can include cluster intensity, cluster offset, and other metrics, including. but not limited to, any of the cluster metrics 506 as described above. In turn, the insert length prediction model 906 processes or analyzes the training cluster metrics 903 (according to its internal model parameters) to generate a predicted insert length 908 (“L”). Indeed, the insert length prediction model 906 generates the predicted insert length 908 from the training nucleotide read 902 to use in a comparison 910. To elaborate, the insert length prediction system 106 performs the comparison 910 to compare the predicted insert length 908 with a ground truth insert length 912. Indeed, the insert length prediction system 106 identifies or determines the ground truth insert length 912 from a set of training data as a true insert length corresponding to the training nucleotide read 902.

Accordingly, the insert length prediction system 106 performs the comparison 910 to compare the ground truth insert length 912 with the predicted insert length 908. In some cases, the insert length prediction system 106 determines an error or a measure of loss between the predicted insert length 908 and the ground truth insert length 912 by performing the comparison 910. For instance, in cases where the insert length prediction model 906 is an ensemble of gradient boosted trees, the insert length prediction system 106 utilizes a mean squared error loss function (e.g., for regression) and/or a logarithmic loss function (e.g., for classification) as the loss function for the comparison 910. Accordingly, the insert length prediction system 106 determines a measure of loss associated with the predicted insert length 908 and/or the insert length prediction model 906.

In some embodiments, the insert length prediction system 106 utilizes training data from Phi-X to train the insert length prediction model 906. To elaborate, the insert length prediction system 106 determines Phi-X data, such as the training nucleotide read 902 and/or the ground truth insert length 912 from the well-mapped and well-understood bacteriophage as training data. In some cases, the insert length prediction system 106 can use the Phi-X training data for offline training and/or for on-the-fly online training (e.g., during implementation), where the ground truth insert length 912 comes from a known insert for Phi-X. Additionally or alternatively, the insert length prediction system 106 can use inserted sequences (e.g., additional or exogenous genomic sequences added to clusters in a flow cell or other nucleotide-sample slide) as training data from a Phi-X spike in for non-Phi-X genomic samples.

As further illustrated in FIG. 9, the insert length prediction system 106 performs parameter adjustment 914. In particular, the insert length prediction system 106 adjusts model parameters to fit the insert length prediction model 906 to the training data based on the comparison 910. For instance, the insert length prediction system 106 performs modifications or adjustments to the insert length prediction model 906 to reduce the measure of loss from one or more loss functions for a subsequent training iteration.

For gradient boosted trees, for example, the insert length prediction system 106 trains the insert length prediction model 906 on the gradients of the errors determined by the loss function of the comparison 910. For instance, the insert length prediction system 106 solves a convex optimization problem (e.g., of infinite dimensions) while regularizing the objective to avoid overfitting. In certain implementations, the insert length prediction system 106 scales the gradients to emphasize corrections to under-represented classes (e.g., where there are significantly more true positives than false positives).

In some embodiments, the insert length prediction system 106 adds a new weak learner (e.g., a new boosted tree) to the insert length prediction model 906 for each successive training iteration as part of solving the optimization problem. For example, the insert length prediction system 106 finds a feature (e.g., a cluster metric or an insert length) that minimizes a loss from a loss function and either adds the feature to the current iteration's tree or starts to build a new tree with the feature.

In addition or in the alternative to gradient boosted decision trees, the insert length prediction system 106 trains a logistic regression to learn parameters for generating one or more variant-call classifications such as a true-positive classification. To avoid overfitting, the insert length prediction system 106 further regularizes based on hyperparameters such as the learning rate, stochastic gradient boosting, the number of trees, the tree-depth(s), complexity penalization, and L1/L2 regularization.

In embodiments where the insert length prediction model 906 is a neural network, the insert length prediction system 106 performs the parameter adjustment 914 by modifying internal parameters (e.g., weights) of the insert length prediction model 906 to reduce the measure of loss for a loss function. Indeed, the insert length prediction system 106 modifies how the insert length prediction model 906 analyzes and passes data between layers and neurons by modifying the internal network parameters. Thus, over multiple iterations, the insert length prediction system 106 improves the accuracy of the insert length prediction model 906.

Indeed, in some cases, the insert length prediction system 106 repeats the training process illustrated in FIG. 9 for multiple iterations. For example, the insert length prediction system 106 repeats the iterative training by selecting a new training nucleotide read along with a corresponding ground truth insert length. The insert length prediction system 106 further generates a new predicted insert length for each iteration based on the new training nucleotide read. As described above, the insert length prediction system 106 also compares the new predicted insert length and the new ground truth insert length at each iteration. In some cases, the insert length prediction system 106 further performs the parameter adjustment 914 for each iteration, iteratively updating model parameters to improve prediction accuracy. The insert length prediction system 106 repeats this process until the insert length prediction model 906 generates predicted insert lengths that satisfy a threshold measure of loss.

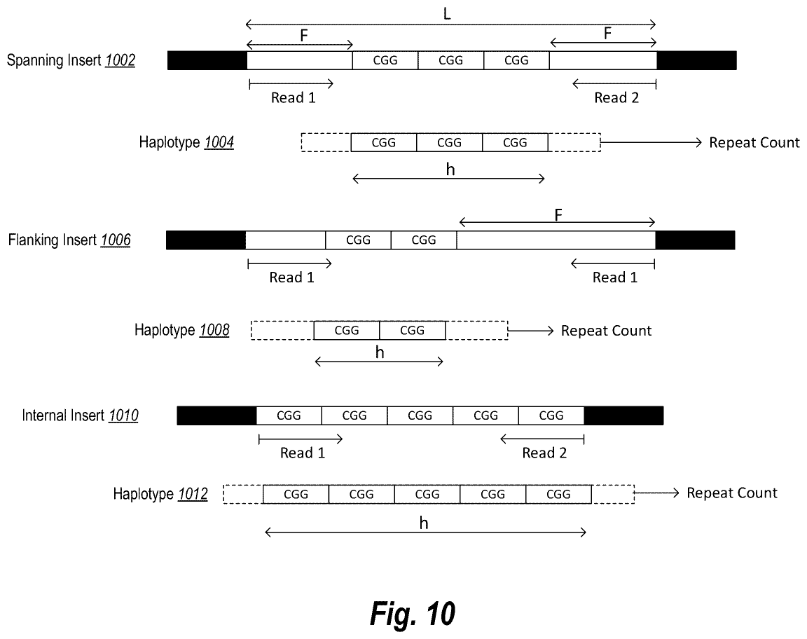

As noted above, in certain described embodiments, the insert length prediction system 106 determines genotype calls for genomic coordinates of a sample genomic sequence based on a predicted insert length. In particular, the insert length prediction system 106 determines a genotype call (e.g., a variant call) for a genomic coordinate as informed by a predicted insert length generated using primary analysis metrics (as described above). For example, the insert length prediction system 106 determines a genotype call (e.g., a variant call) in the form of a tandem repeat call (e.g., a VNTR) by determining a number of repeat units within a repeat region corresponding to (e.g., encompassing or including) a genomic coordinate. FIG. 10 illustrates an example diagram for determining a repeat count for VNTR based on different types of inserts (or sample genomic sequences) in accordance with one or more embodiments.

As illustrated in FIG. 10, the insert length prediction system 106 determines repeat counts for haplotypes corresponding to different types of overlapping inserts, such as spanning inserts, flanking inserts, and internal inserts. Indeed, the insert length prediction system 106 can determine or classify an insert (and/or nucleotide read pairs corresponding to an insert) into one or more possible categories or types: overlapping, spanning, or flanking (or into additional, more granular categories). The insert length prediction system 106 can further determine a range of candidate haplotype lengths from (i) a complete deletion of a genomic region (which would result in a shortest length) to (ii) a reference haplotype to (iii) a large insertion length (which would result in a longest length). For each possible length, the insert length prediction system 106 can determine a likelihood or a probability that a read fragment belongs to each of the possible categories (summing up the likelihood of each). The insert length prediction system 106 can further multiply the likelihoods of reads in each category by the priors for each category and sum the results together to determine a final posterior probability for the haplotype length. In addition, the insert length prediction system 106 can select a haplotype with a highest posterior probability as corresponding to the predicted insert length.

To elaborate, the insert length prediction system 106 determines or identifies a set of haplotypes corresponding to a genomic coordinate or a genomic region of a sample genomic sequence. The insert length prediction system 106 can further determine haplotype probabilities for each of the haplotypes within the set, where the haplotype probabilities reflect or define likelihoods that respective haplotypes in the set match or correspond to the coordinate/region. For instance, the insert length prediction system 106 determines a haplotype probability that a haplotype having a particular size or length (e.g., a number of nucleobases) matches a size or length of a repeat region within an insert (or sequence). The insert length prediction system 106 repeats this determination for all haplotypes in the set and selects a highest probability haplotype as corresponding to the insert having a predicted insert length (e.g., the haplotype whose probability reflects a size closest to the predicted insert length).

As just indicated, in some embodiments, the insert length prediction system 106 can determine prior haplotype probabilities and posterior haplotype probabilities that a genomic sample comprises different candidate haplotypes of different lengths (e.g., numbers of nucleobases) with respect to a tandem repeat region as the basis for determining genotype probabilities for a given genomic coordinate and (based on such genotype probabilities) determining a genotype call. In some embodiments, for instance, the insert length prediction system 106 determines a first prior haplotype probability, a second prior haplotype probability, a third prior haplotype probability, and/or an nth prior haplotype probability of the genomic sample comprising a first candidate haplotype of a first length, a second candidate haplotype of a second length, a third candidate haplotype of a third length, and/or an nth candidate haplotype of an nth length with respect to a tandem repeat region based on a respective distribution of predicted insert lengths for a nucleotide read pair corresponding to a genomic coordinate. Such prior haplotype probabilities can sum to one. The insert length prediction system 106 further determines a first posterior haplotype probability, a second posterior haplotype probability, a third posterior haplotype probability, and/or an nth posterior haplotype probability of the genomic sample comprising a first candidate haplotype of a first length, a second candidate haplotype of a second length, a third candidate haplotype of a third length, and/or an nth candidate haplotype of an nth length with respect to a tandem repeat region based on (i) a respective distribution of predicted insert lengths for the nucleotide read pair corresponding to the genomic coordinate, (ii) the nucleotide read part, and (iii) the prior haplotype probabilities. Such posterior haplotype probabilities can likewise sum to one.

Based on the determined posterior haplotype probabilities, the insert length prediction system 106 further determines genotype probabilities and a corresponding genotype call. While the paragraph below describe prior and posterior haplotype probabilities with respect to spanning inserts, flanking inserts, and internal inserts with respect to a tandem repeat region, the insert length prediction system 106 can also use more granular or specific categories for candidate haplotypes with respect to the tandem repeat region and determine corresponding haplotype probabilities.

As shown in FIG. 10, the insert length prediction system 106 determines or selects a haplotype 1004 for a spanning insert 1002. As part of the determination process, for the spanning insert 1002 of length L (where L is the insert length or insert size), the insert length prediction system 106 determines that there are L−h−2F+1 possible positions for the spanning insert 1002, where h represents the haplotype length (including a number of nucleobases in the haplotype 1004 with or without flanking regions, as indicated by the dashed lines) and F represents a threshold (e.g., minimum) number of flanking nucleobases on either side of the spanning insert 1002 to be considered “spanning.”

As also shown in FIG. 10, the insert length prediction system 106 determines or selects a haplotype 1008 for a flanking insert 1006. As part of the determination process, for the flanking insert 1006 of length L, the insert length prediction system 106 determines that there are 2L−2F−2K+2 possible positions for the flanking insert 1006 if L−2F<h, and that there are 2h+2F−2K possible positions for the flanking insert 1006 if L−2F≥h, where K is the number of overlapping nucleobases required for an insert to qualify as overlapping a repeat region and the other terms are as defined above.

As further shown, the insert length prediction system 106 determines or selects a haplotype 1012 for an internal insert 1010. As part of the determination process, for the internal insert 1010 of length L, the insert length prediction system 106 determines that there are h−L+2F−1 possible positions for the internal insert 1010. If the insert length prediction system 106 determines that L>h+2F−2, then the insert length prediction system 106 also determines that it is impossible for the internal insert 1010 to be in-repeat.

In some embodiments, to determine VNTRs for one or more of the spanning insert 1002, the flanking insert 1006, and/or the internal insert 1010, the insert length prediction system 106 utilizes some baseline parameters. For example, the insert length prediction system 106 determines or assures that, for a given insert, L>2F, h>2K, and L≥K+F, where the terms are defined above. In some cases, K can be zero if it is sufficient for a nucleotide read fragment to be immediately adjacent to a repeat region, or negative if some distance between the read fragment and the repeat region is allowed.

According to the above parameters for the spanning insert 1002, the flanking insert 1006, and the internal insert 1010, the insert length prediction system 106 defines positions of inserts according to the following functions:

$$overlapPositions ( L , h ) = max \{ 0 , L + h - 2 K + 1 \}$$

$$spanPositions ( L , h ) = max \{ 0 , L - h - 2 F + 1 \}$$

$$flankPositions ( L , h ) = max \{ \begin{matrix}2 h + 2 F - 2 K & if L - h - 2 F \geq 0 \\ 2 L - 2 F - 2 K + 2 & otherwise\end{matrix} \}$$

$$internalPositions ( L , h ) = max \{ 0 , h + L + 2 f - 1 \}$$

where overlapPositions(L, h) defines coordinates of an overlapping insert, spanPositions(L, h) defines coordinates of a spanning insert, flankPositions(L, h) defines coordinates of a flanking insert, and internalPositions(L, h) defines coordinates of an internal insert.

In some embodiments, at a given locus or genomic coordinate of interest (e.g., a genomic coordinate with a sample genomic sequence), the insert length prediction system 106 identifies H haplotypes, each having a haplotype length (or implied array size) of h. The insert length prediction system 106 further determines a posterior probability for all possible genotypes G (where G is a haplotype pair, G={h₁, h₂}), as given by the function:

$$P ( G \vert R ) ∝ P ( R \vert G ) P ( G )$$

where R represents a read pair pileup. In some cases, the insert length prediction system 106 determines the genotype prior P(G) based on population frequencies of known per-locus haplotypes, also accounting for genome-wide estimates of variant rates for previously unknown variants.

In one or more implementations, the insert length prediction system 106 determines P(R|G) (or P(R|GN) using the following function:

$$P ( R \vert G ) = {\prod }_{i} P ( {r}_{i} \vert G , map )$$

where N is the number of read fragments overlapping a repeat region and where map represents the occurrence where a current read is mapped to a current locus or genomic coordinate. Given a read pair rᵢ, an insert length L, and a haplotype length h, the insert length prediction system 106 can thus determine if the number repeat-overlapping nucleobases in the read pairs is compatible with the insert length and the haplotype length. Specifically, the insert length prediction system 106 enforces the following conditions as part of a compatibility function C(rᵢ, h₁, L): i) if exactly one read overlaps the repeat, the number of repeat-overlapping nucleobases must not exceed the haplotype length, ii) if both reads overlap the repeat, the number of unique repeat-overlapping bases plus the inner distance (e.g., the insert size/insert length minus the two read lengths or zero if the reads overlap), iii) if the insert spans the repeat, the insert size/insert length must be equal to Lₕᵢ, iv) if a single read spans the repeat, the number of repeat-overlapping nucleobases in the read must equal the haplotype length, and v) if any of i)-iv) are violated, then the read pair is incompatible, otherwise it is compatible.

As indicated above, the insert length prediction system 106 distinguishes between spanning, flanking, and internal insert in determining genotype probabilities. In addition, the insert length prediction system 106 utilizes a mapping model to predict mappability as a function of features that are specific to the position and the insert type. Based on the mappability indicating read mapping, the insert length prediction system 106 determines genotype probabilities (or read pair probabilities) for genotypes, as given by the following function:

$$P ( {r}_{i} \vert G , map ) = \{ \begin{matrix}P ( span \vert G , map ) P ( {r}_{i} \vert G , span ) & if {r}_{i} is a spanning fragment \\ P ( flank \vert G , map ) P ( {r}_{i} \vert G , flank ) & if {r}_{i} is a flanking fragment \\ P ( internal \vert G , map ) P ( {r}_{i} \vert G , internal ) & if {r}_{i} is an internal fragment\end{matrix} \}$$

$$where$$

$$P ( span \vert G , map ) = \frac{P ( map \vert span ) P ( span \vert G )}{P ( map \vert G )}$$

$$P ( flank \vert G , map ) = \frac{P ( map \vert flank ) P ( flank \vert G )}{P ( map \vert G )}$$

$$P ( internal \vert G , map ) = \frac{P ( map \vert internal ) P ( internal \vert G )}{P ( map \vert G )}$$

$$and$$

$$P ( map \vert G ) = P ( map \vert span ) P ( span \vert G ) + P ( map \vert flank ) P ( flank \vert G ) + P ( map \vert internal ) P ( internal \vert G )$$

and where P(map|span/flank/internal) represents predicted position-specific mappabilities for the three respective insert types, and where P(span/flank/internal|G) are internal priors, which are elucidated hereafter.

As just indicated, the insert length prediction system 106 determines internal priors for the spanning insert 1002, the flanking insert 1006, and the internal insert 1010. Specifically, the insert length prediction system 106 determines priors as functions of the genotype G, where if h₁ is short and h₂ is very long (e.g., unlikely to be spanned by a typical insert), then G={h₁, h₂} should result in close to half of overlapping insert spanning the repeat, whereas G={h₁, h₁} should result in almost all of the overlapping inserts spanning the repeat. Accordingly, in some embodiments, the prior probability of the insert type (or read type) depends on the insert length (e.g., the predicted insert length determined via primary analysis metrics, as described above), and the insert length prediction system 106 determines internal priors for insert types according to the following functions:

$$P ( span \vert G ) = {\sum }_{L} P ( L ) P ( span \vert GL )$$

$$P ( flank \vert G ) = {\sum }_{L} P ( L ) P ( flank \vert GL )$$

$$P ( internal \vert G ) = {\sum }_{L} P ( L ) P ( internal \vert GL )$$

where P(L) represents a probability distribution of predicted insert lengths (or a predicted insert length as represented by a probability distribution of insert lengths), as determined from primary analysis metrics according to the description above.

Given L, the probabilities also depend on the lengths of the two repeat alleles, as given by the following functions:

$$P ( span \vert GL ) = P ( span \vert {h}_{1} L ) P ( {h}_{1} \vert GL ) + P ( span \vert {h}_{2} L ) P ( {h}_{2} \vert GL )$$

$$P ( flank \vert GL ) = P ( flank \vert {h}_{1} L ) P ( {h}_{1} \vert GL ) + P ( flank \vert {h}_{2} L ) P ( {h}_{2} \vert GL )$$

$$P ( internal \vert GL ) = P ( internal \vert {h}_{1} L ) P ( {h}_{1} \vert GL ) + P ( internal \vert {h}_{2} L ) P ( {h}_{2} \vert GL ) .$$

In one or more embodiments, the probability that an insert is templated on a specific haplotype depends on the number of possible overlapping insert positions for each of the two haplotypes. Accordingly, the insert length prediction system 106 can determine a haplotype probability for each haplotype of a set of haplotypes. For instance, the insert length prediction system 106 can determine a haplotype probability that a haplotype of a certain length matches or corresponds to a insert having a particular insert size. In some cases, the insert length prediction system 106 utilizes the following function:

$$P ( {h}_{1} \vert GL ) = \frac{L + {h}_{1} - 2 K + 1}{( L + {h}_{1} - 2 K + 1 ) + ( L + {h}_{2} - 2 K + 1 )} = \frac{L + {h}_{1} - 2 K + 1}{2 L + {h}_{1} + {h}_{2} - 4 K + 2}$$

In some embodiments, using variations on this equation, the insert length prediction system 106 determines haplotype probabilities for a set of haplotypes corresponding to a genomic coordinate and selects a haplotype with a highest haplotype probability as matching or representing the genomic coordinate. For instance, the insert length prediction system 106 selects the haplotype 1004 as the highest probability haplotype for the spanning insert 1002, selects the haplotype 1008 as the highest probability haplotype for the flanking insert 1006, and selects the haplotype 1012 as highest probability haplotype for the internal insert 1010. Additionally, the insert length prediction system 106 can determine a repeat count for repeat units of a VNTR based on the haplotype. For example, based on a k-mer length or a motif length of a repeat unit within a repeat region, and further based on a length of a haplotype the insert length prediction system 106 can determine a repeat count for the VNTR (e.g., by dividing the haplotype length by the repeat unit length and rounding (down) to the nearest integer).

In one or more embodiments, the insert length prediction system 106 further determines prior probabilities for the spanning insert 1002, the flanking insert 1006, and the internal insert 1010. For example, the insert length prediction system 106 determines a spanning prior as a probability that an insert overlapping a given haplotype will span that haplotype, as given by the function:

$$P ( span \vert hL ) = \frac{spanPositions ( L , h )}{overlapPositions ( L , h )}$$

$$where \frac{spanPositions ( L , h )}{overlapPositions ( L , h )}$$

represents a fraction of overlap positions/coordinates that are also spanning positions/coordinates. In addition, the insert length prediction system 106 determines a flanking prior as a probability that an insert overlapping a given haplotype will flank the haplotype, as given by the function:

$$P ( flank \vert hL ) = \frac{flankPositions ( L , h )}{overlapPositions ( L , h )}$$

$$where \frac{flankPositions ( L , h )}{overlapPositions ( L , h )}$$

represents a fraction of overlap positions that are also flanking positions. Further, the insert length prediction system 106 determines an internal prior as a probability that an insert overlapping a given haplotype will be internal to that haplotype, as given by the function:

$$P ( internal \vert hL ) = \frac{internalPositions ( L , h )}{overlapPositions ( L , h )}$$

$$where \frac{internalPositions ( L , h )}{overlapPositions ( L , h )}$$

represents a fraction of overlap positions that are also internal positions.

As indicated above, in one or more embodiments, the insert length prediction system 106 determines genotype probabilities for different insert types, including the spanning insert 1002, the flanking insert 1006, and the internal insert 1010. More particularly, to determine the fragment likelihood for the spanning insert 1002, the insert length prediction system 106 sums over all possible insert lengths, as given by the function:

$$P ( {r}_{i} \vert G , span ) = {\sum }_{L} P ( L ) P ( {r}_{i} \vert GL , span )$$

where P(L|G,span)=P(L), and P(L) is the probability distribution of predicted insert lengths determined using primary (and/or other) metrics, as described herein. In certain cases, the insert length prediction system 106 disregards the possibility that restricting the spanning reads would result in a different insert size distribution, and the insert length prediction system 106 further disregards potential dependence on the genotype. Additionally, the insert length prediction system 106 determines the weighted sum of per-haplotype likelihoods according to the function:

$$P ( {r}_{i} \vert GL , span ) = P ( {r}_{i} \vert {h}_{1} L , span ) P ( {h}_{1} \vert GL , span ) + P ( {r}_{i} \vert {h}_{2} L , span ) P ( {h}_{2} \vert GL , span )$$

where the likelihood terms are nonzero only when the insert size matches the length implied by the read pair and haplotype. In such cases, the insert length prediction system 106 determines that the read is mapped to a single unique position out of the possible spanning positions, resulting in a likelihood of one of the number of spanning positions, according to the following function:

$$P ( {r}_{i} \vert {h}_{1} L , span ) = \frac{C ( {r}_{i} , {h}_{1} , L )}{spanPositions ( L , {h}_{1} )}$$

where h₂ has a similar function, and where C(rᵢ, h₁, L) is a compatibility function defined above.

In some embodiments, the compatibility function C(rᵢ, h₁, L) is nonzero only in cases where the denominator is positive and zero otherwise, so the insert length prediction system 106 ignores or excludes cases where L−h₁−2F+1<0. Additionally, the insert length prediction system 106 combines the genotype probability function with the sum over L to determine a modified genotype probability function given by:

$$P ( {r}_{i} \vert G , span ) = P ( {L}_{{h}_{1} i} ) \frac{P ( {h}_{1} \vert {GL}_{{h}_{1} i} , span )}{{L}_{{h}_{1} i} - {h}_{1} - 2 F + 1} + P ( {L}_{{h}_{2} i} ) \frac{P ( {h}_{2} \vert {GL}_{{h}_{2} i} , span )}{{L}_{{h}_{2} i} - {h}_{2} - 2 F + 1}$$

where terms that are incompatible due to L≠Lₕᵢ are removed, and where terms that are incompatible due to having too many bases overlapping the repeat are set to zero. Note that for a homozygous genotype where h₁=h₂, the two terms in this function are equal, and P(h₁|GLₕ_{₁}ᵢ, span)=0.5, so the insert length prediction system 106 can utilize a simplified version of the modified genotype probability function for homozygous genotypes, as given by:

$$P ( {r}_{i} \vert G , span ) = \frac{P ( {L}_{{h}_{1} i} )}{{L}_{{h}_{1} i} - {h}_{1} - 2 F + 1} .$$

In one or more embodiments, the insert length prediction system 106 quantifies weights (e.g., probabilities that a spanning read is templated on a specific haplotype) by treating the diploid genome as a single haploid genome of twice the length, where the two copies have been concatenated such that there are two possible mappings of each read pair. In some cases, the probabilities are not equal because the copies are not necessarily equal in length. Rather, the insert length prediction system 106 determines the probability for a given haplotype as equal to the number of possible spanning insert positions for that haplotype, divided by the total number of spanning insert positions, as represented by the following functions:

$$P ( {h}_{1} \vert {GL}_{{h}_{1} i} , span ) = \frac{{L}_{{h}_{1} i} - {h}_{1} - 2 F + 1}{spanPositions ( {L}_{{h}_{1} i} , {h}_{1} ) + spanPositions ( {L}_{{h}_{1} i} , {h}_{2} )}$$

$$and$$

$$P ( {h}_{2} \vert {GL}_{{h}_{2} i} , span ) = \frac{{L}_{{h}_{2} i} - {h}_{2} - 2 F + 1}{spanPositions ( {L}_{{h}_{2} i} , {h}_{1} ) + spanPositions ( {L}_{{h}_{2} i} , {h}_{2} )} .$$

As shown above, the insert length prediction system 106 determines four versions of the spanPositions term. When the L and h arguments match, the spanPositions count is guaranteed to be positive, but the same is not true when the L and h arguments do not match (i.e., it is not a guarantee that Lₕ_{₁}ᵢ−h₂−2F+1>0). In some embodiments, the insert length prediction system 106 thus combines the haplotype probabilities to determine a genotype probability for the spanning insert 1002, as given by the function:

$$P ( {r}_{i} \vert G , span ) = \frac{P ( {L}_{{h}_{1} i} )}{spanPositions ( {L}_{{h}_{1} i} , {h}_{1} ) + spanPositions ( {L}_{{h}_{1} i} , {h}_{2} )} + \frac{P ( {L}_{{h}_{2} i} )}{spanPositions ( {L}_{{h}_{2} i} , {h}_{1} ) + spanPositions ( {L}_{{h}_{2} i} , {h}_{2} )} .$$

The insert length prediction system 106 can further determine genotype probabilities for the flanking insert 1006 and the internal insert 1010 as well, using the same functions as described for the spanning insert 1002 but with flankPositions and internalPositions to replace the spanPositions, respectively. For the flanking insert 1006 and the internal insert 1010, there are likely to be many nonzero terms (instead of at most two as for the spanning insert 1002), and the insert length prediction system 106 determines the number of repeat-overlapping to be more relevant in these circumstances.

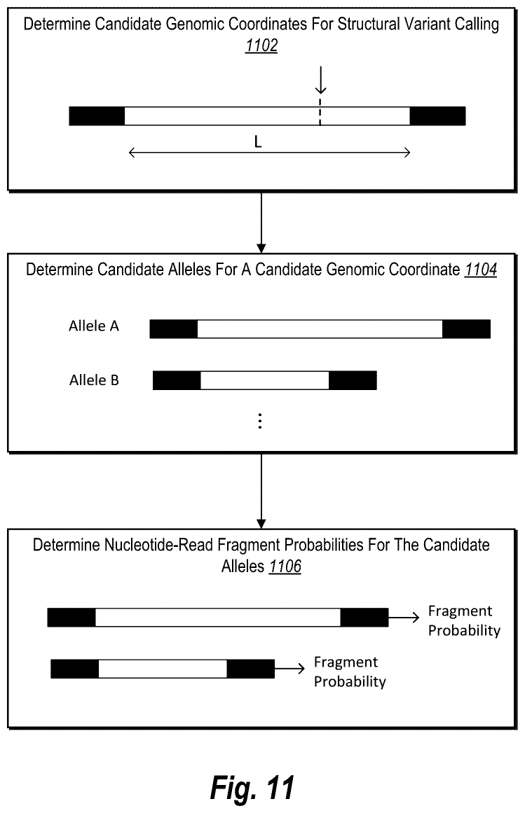

As mentioned, in certain embodiments, the insert length prediction system 106 determines a structural variant corresponding to a genomic coordinate of a sample genomic sequence. In particular, the insert length prediction system 106 determines a structural variant based on a predicted insert length for the sample genomic coordinate (e.g., for an insert). FIG. 11 illustrates an example diagram for determining a structural variant for a genomic coordinate based on a predicted insert length.

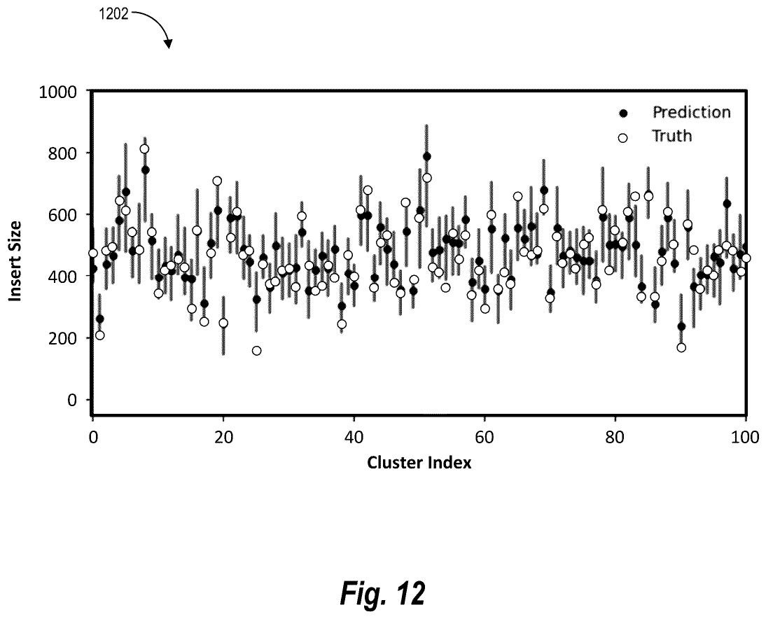

As illustrated in FIG. 11, the insert length prediction system 106 performs an act 1102 to determine candidate genomic coordinates for structural variants. More particularly, the insert length prediction system 106 determines candidate genomic coordinates by utilizing a structural variant discovery or detection process. As part of structural variant discovery, the insert length prediction system 106 determines candidate structural variants for read pairs with an unexpected orientation, chimeric mapping, or an anomalously large insert size (anomalous insert sizes are discussed in further detail in relation to FIG. 12 below). For example, the insert length prediction system 106 determines large insert sizes by determining the extent of the read pair in reference coordinates and comparing the predicted insert length to the expected insert length. Specifically, the insert length prediction system 106 can determine whether predicted insert length differs by a threshold number of nucleobases from an expected insert length.

In some embodiments, the insert length prediction system 106 determines candidate genomic coordinates for structural variants by identifying nucleotide reads exhibiting abnormal alignments or structural variant alignment tags. In some cases, the insert length prediction system 106 can identify nucleotide reads exhibiting abnormal alignments by identifying a cluster of nucleotide read alignments with masked read fragments or pairs of read fragment (e.g., partial read) alignments falling below or exceeding a threshold insert size. In some embodiments, the threshold insert size is fixed. In alternative embodiments, the threshold insert size is dynamic and/or model based (e.g., mixed Gaussian model).

To illustrate, in some embodiments, the insert length prediction system 106 identifies a cluster of nucleotide read alignments with masked read fragments or nucleobases that satisfy or exceed a threshold number of nucleobases (e.g., 15, 20, 35 nucleobases for each read in the cluster of nucleotide read alignments). In some cases, the nucleotide reads from the cluster include masked read fragments that align with an alternate contiguous sequence. In one or more embodiments, the insert length prediction system 106 identifies a genomic coordinate for a corresponding primary contiguous sequence as a candidate genomic coordinate for a candidate structural variant.

Additionally, in some cases, the insert length prediction system 106 determines that nucleotide reads exhibit abnormal alignments by identifying nucleotide reads with an estimated or predict insert size falling below or exceeding a threshold insert size. For instance, the threshold insert size can be fixed or can be dynamic and change based on the dataset and/or the genomic sample. To illustrate, in a read data set for a given genomic sample, the insert length prediction system 106 determines a distribution of (predicted) insert sizes corresponding to paired-end reads by using one or more primary metrics and an insert length prediction model, as described above.

Based on the distribution of insert sizes and the standard deviation, the insert length prediction system 106 can identify insert sizes in an alignment file (e.g., SAM file) that fall outside of the standard deviation. When, for instance, the insert length prediction system 106 determines a mean insert size of 500 base pairs (or nucleobases) for a genomic sample with a standard deviation of 100 base pairs (or nucleobases), the insert length prediction system 106 identifies, from the genomic sample's alignment file, a determined insert size for paired-end reads outside of the standard deviation—at only 100 base pairs or at 1000 base pairs, for example—as falling below or exceeding a threshold insert size.

As mentioned above, the threshold insert size can be dynamic. More specifically, in some embodiments, the insert length prediction system 106 can determine a threshold insert size can based on: (i) the size distribution of a genomic sample sequence from a sample library fragment and (ii) the actual insert size of the genomic sample sequence corresponding to paired-end reads. For example, in certain cases, the insert length prediction system 106 fits the actual insert size to a fitting model (e.g., mixed Gaussian). Based on the fitting model, the insert length prediction system 106 can identify pairs of nucleotide read fragment alignments with an insert size that falls below or exceeds the threshold insert size. For example, if the expected insert size distribution is 500 base pairs±100 base pairs, nucleotide read fragment alignments with an insert size of 600 base pair fragments do not exceed the threshold insert size. Conversely, if the expected insert size distribution is 250 base pairs±50, a pair of read fragments with an insert size of 600 base pairs would exceed the threshold insert size, indicate an abnormal alignment, and provide support for a structural variant haplotype. Indeed, the insert length prediction system 106 can detect that a genomic coordinate is a candidate for a structural variant based on a predicted insert length differing from an expected insert length by at least a threshold number of nucleobases.

As further illustrated in FIG. 11, the insert length prediction system 106 performs an act 1104 to determine candidate alleles for a candidate genomic coordinate for structural variant calling (or candidate structural-variant genomic coordinate). To elaborate, the insert length prediction system 106 determines or identifies a set of alleles corresponding to a genomic coordinate identified as a candidate for exhibiting, reflecting, or being a part of, a structural variant. For instance, the insert length prediction system 106 accesses a repository or a set of known alleles that reflect candidate nucleobase sequences for a region of a sample genomic sequence (or an insert) that includes a candidate genomic coordinate for a structural variant. In some cases, the insert length prediction system 106 determines candidate alleles in the form of a structural-variant-candidate allele and a reference allele for a candidate structural variant (breakend) coordinate. As shown, the insert length prediction system 106 identifies Allele A and Allele B, each of different lengths, as candidate alleles for a candidate genomic coordinate, where Allele A may represent a reference allele and Allele B may represent a candidate structural variant allele (e.g., a deletion, as it is shorter in length).

As further illustrated in FIG. 11, the insert length prediction system 106 performs an act 1106 to determine nucleotide-read fragment probabilities for the candidate alleles. For example, the insert length prediction system 106 determines a nucleotide-read fragment probability for a given candidate allele by determining a likelihood that a nucleotide-read fragment supports (or reflects or includes nucleobases of) a given allele. In some cases, the insert length prediction system 106 determines the nucleotide-read fragment probabilities based on a predicted insert length (or a distribution of predicted insert lengths). In some embodiments, the insert length prediction system 106 identifies a set of candidate alleles for a genomic coordinate, where the set of candidate alleles includes one allele corresponding to (e.g., within a threshold number of nucleobases of) a predicted insert length and another allele that is inconsistent with (e.g., outside of a threshold number of nucleobases of) the predicted insert length. The insert length prediction system 106 further determines nucleotide-read fragment probabilities for the candidate alleles by determining likelihoods of nucleotide-read fragments from the set of nucleotide-read fragments supporting respective candidate alleles.

In some embodiments, the insert length prediction system 106 generates nucleotide-read fragment probabilities based on incorporating the allele frequency corresponding to a structural variant haplotype. In particular, the insert length prediction system 106 can score a candidate structural variant call or a candidate allele by utilizing a diploid scoring model. For instance, the diploid scoring model generates diploid genotype probabilities for each candidate structural variant at a given candidate genomic coordinate. In some embodiments, for scoring purposes, the insert length prediction system 106 approximates the candidate structural variants based on candidate alleles exhibited within the filtered set of nucleotide reads. In such cases, the insert length prediction system 106 applies a model with a single alternate allele.

For example, for candidate alleles A={r, x}, where A represents possible combinations of an allele r representing a reference allele and x representing an alternate allele, the insert length prediction system 106 can apply a diploid scoring model for candidate genotypes that include or exclude candidate alleles for a structural variant at a candidate genomic coordinate. In some cases, the insert length prediction system 106 restricts the genotypes for each candidate allele to G={rr, rx, xx}, where G represents the genotype, rr represents alleles for a homozygous genotype of reference alleles from a primary contiguous sequence, rx represents alleles for a heterozygous genotype of one reference allele and one alternate allele from alternate contiguous sequence, and xx represents alleles with a homozygous genotype of alternate alleles from an alternate contiguous sequence (e.g., representing a structural variant).

In some embodiments, the insert length prediction system 106 can generate a nucleotide-read fragment probability for a candidate allele by generating a posterior probability for a candidate genotype based on a prior probability for the candidate genotype. In one or more embodiments, the insert length prediction system 106 solves the posterior probability over G according to the following equation: P(G|D)∝P(D|G)P(G), where D represents all the supporting read fragments (or supporting nucleotide reads) for either candidate allele, P(G|D) represents the posterior probability for a genotype given the supporting read fragments (or supporting nucleotide reads) for either candidate allele, P(D|G) represents the probability of the supporting read fragments (or supporting nucleotide reads) for either allele given the genotype, and P(G) represents the prior probability for the genotype. Such supporting read fragments or supporting nucleotide reads can come from the filtered set of nucleotide reads. In one or more embodiments, the insert length prediction system 106 determines the prior probability P(G) according to the following equation:

$$P ( G ) = \{ \begin{matrix}{θ}_{SV} & if rx \\ {θ}_{SV} / 2 & if xx \\ 1 - {θ}_{SV} 3 / 2 & if rr\end{matrix}$$

where θ_{SV} represents an allele frequency for a structural variant heterozygosity. In some embodiments, the allele frequency for structural variant heterozygosity is set at a default of 1×10⁻⁵. By contrast, in some embodiments, the insert length prediction system 106 (i) identifies, from a haplotype database (e.g., Structural Variation Data Hub from the National Institutes of Health) or other population data, an allele frequency for a candidate structural variant haplotype represented by a given alternate contiguous sequence and (ii) uses the identified allele frequency for the candidate structural variant haplotype as θ_{SV} for structural variant heterozygosity. In one or more implementations, the insert length prediction system 106 computes the likelihood P(D|G) by assuming that each supporting read fragment (or supporting nucleotide read) represents an independent observation of the genomic sample. For instance, in some embodiments, the insert length prediction system 106 determines P(D|G) according to the following equation:

$$P ( D \vert G ) = {\prod }_{d ∈ D} P ( d \vert G )$$

where d∈D represents an independent observation of the supporting read fragments (or supporting nucleotide reads) of the genomic sample and P(d|G) represents a read fragment likelihood (or nucleotide read likelihood). As further indicated below in some embodiments, the insert length prediction system 106 determines a nucleotide-read fragment probability by summing together the read fragments (or nucleotide reads) according to the following equation:

$$P ( d \vert G ) = {\sum }_{a ∈ A} P ( d \vert a ) P ( a \vert G )$$

where a∈A represents an independent observation of the candidate alleles, P(d|a) represents a likelihood for each read fragment (or nucleotide read) to support the given allele, and P(a|G) represents standard diploid variant frequencies from {0,0.5,1}.

In one or more embodiments, the insert length prediction system 106 determines the fragment probability for each of a number of candidate alleles. For instance, the insert length prediction system 106 determines a first set of nucleotide-read fragment probabilities that the fragment of length L supports Allele A, and the insert length prediction system 106 determines a second set of nucleotide-read fragment probabilities that the fragment supports Allele B. In some cases, the insert length prediction system 106 weights the candidate alleles as part of determining the nucleotide-read fragment probabilities, where, for example, alleles that are closer in length to the fragment are weighted more heavily than alleles that have less similar lengths. The insert length prediction system 106 further compares the nucleotide-read fragment probabilities to select a candidate allele with a highest nucleotide-read fragment probability as corresponding to the fragment (or the candidate allele with the most corresponding nucleotide-read fragment probabilities).

To determine a structural variant call for a target genomic region of a genomic sample, the insert length prediction system 106 determines a genotype call based on the fragment probabilities (e.g., posterior genotype probabilities). For instance, in certain implementations, the insert length prediction system 106 selects a genotype (or allele) exhibiting a highest posterior genotype probability (among genotype probabilities) for a target genomic region or genomic coordinate as the genotype call for a genomic sample. If the selected genotype (or allele) exhibits a structural variant, the insert length prediction system 106 generates a structural variant call for the genomic sample at the target genomic region or the target genomic coordinate. Accordingly, the insert length prediction system 106 can generate, for a target genomic region of a genomic sample, a structural variant call based on a candidate alignment of the one or more nucleotide reads with at least part of an alternate contiguous sequence representing a structural variant haplotype. In some cases, the insert length prediction system 106 determines a positive structural variant call identifying a presence of a structural variant or a negative structural variant call identifying an absence of the structural variant.

As mentioned above, in certain described embodiments, the insert length prediction system 106 can detect or determine anomalous values as part of determining genotype calls, such as VNTRs or structural variants. In particular, the insert length prediction system 106 can determine anomalous nucleobase values for specific nucleobase clusters and/or inserts as candidates for exhibiting or including variants, such as VNTRs and/or structural variants. FIG. 12 illustrates an example graph depicting anomalous values on a per-cluster or per-insert scale.

As illustrated in FIG. 12, the graph 1202 depicts predicted insert sizes compared to actual (truth) insert sizes. The gray vertical lines denote a 95% confidence interval where the insert length prediction system 106 determines with 95% confidence that a predicted insert length will fall somewhere along the gray line. As shown, for some clusters or inserts, the insert length prediction system 106 generates some predicted insert lengths where the 95% confidence interval does not encompass the actual insert length and are thus indicated to be anomalous. Thus, by determining predicted insert lengths on a cluster-specific level or an insert-specific level, the insert length prediction system 106 more accurately determines anomalous values for improved accuracy in genotype calling, and particularly variant calling for VNTRs and/or structural variants that incorporate anomalous value detection for candidate variant coordinates. Indeed, the insert length prediction system 106 can detect anomalous values much more accurately than existing sequencing systems that generate predicted insert sizes on an index level across many clusters or inserts, where, for example, certain values that may be anomalous relative to their specific cluster may not be anomalous across the index of many clusters.

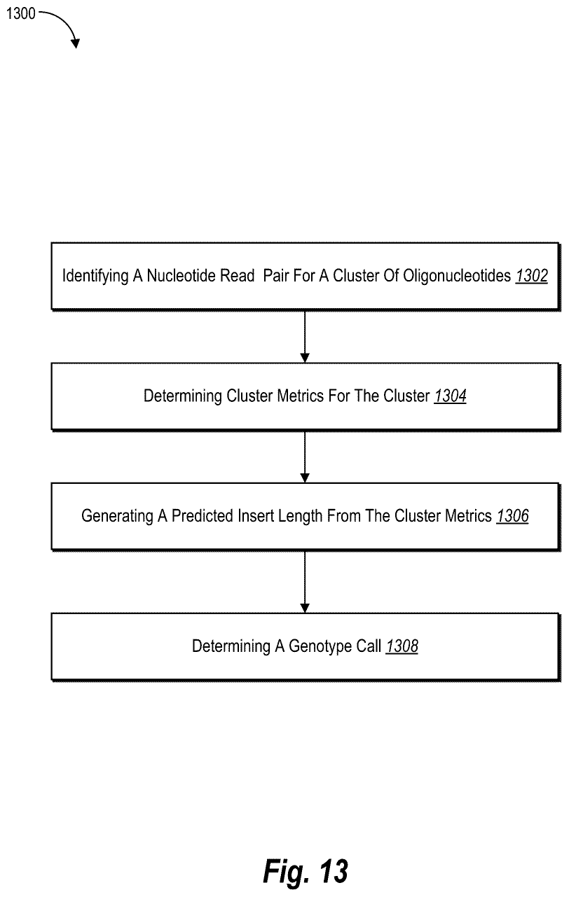

Turning now to FIG. 13, this figure illustrates an example flowchart of a series of acts of determining a predicted insert length of a genomic sequence for mapping and genotype calling. While FIG. 13 illustrates acts according to one or embodiments, alternative embodiments may omit, add to, reorder, and/or modify any of the acts shown in FIG. 13. The acts of FIG. 13 can be performed as part of a method. Alternatively, a non-transitory computer readable storage medium can comprise instructions that, when executed by one or more processors, cause a computing device to perform the acts depicted in FIG. 13. In still further embodiments, a system comprising at least one processor and a non-transitory computer readable medium comprising instructions that, when executed by one or more processors, cause the system to perform the acts of FIG. 13.

As shown in FIG. 13, the series of acts 1300 includes an act 1302 of identifying a nucleotide read pair for a cluster of oligonucleotides. In addition, the series of acts 1300 includes an act 1304 of determining cluster metrics for the cluster. Further, the series of acts 1300 includes an act 1306 of generating a predicted insert length from the cluster metrics. As also shown, the series of acts 1300 includes an act 1308 of determining a genotype call. In some embodiments, the series of acts 1300 includes acts to perform any of the operations described in the following clauses:

CLAUSE 1. A method comprising:
identifying, for a cluster of oligonucleotides corresponding to a sample genomic sequence, a nucleotide read pair comprising a first nucleotide read complementing a first portion of the sample genomic sequence and a second nucleotide read complementing a second portion of the sample genomic sequence;
determining one or more cluster metrics associated with the cluster of oligonucleotides;
generating, using an insert length prediction model to process the one or more cluster metrics, at least one predicted insert length of the sample genomic sequence; and
determining a genotype call for a genomic coordinate within the genomic region based on the at least one predicted insert length.

CLAUSE 2. The method of clause 1, further comprising generating the at least one predicted insert length by determining, using the insert length prediction model, one or more of a distribution of predicted insert lengths of the sample genomic sequence or a mean predicted insert length from the distribution of predicted insert lengths.

CLAUSE 3. The method of any of clauses 1-2, further comprising determining the distribution of predicted insert lengths by determining one or more of a parametric distribution of predicted insert lengths, a non-parametric distribution of predicted insert lengths, an expectile of predicted insert lengths, or a quantile of predicted insert lengths.

CLAUSE 4. The method of any of clauses 1-3, further comprising generating the at least one predicted insert length by utilizing the insert length prediction model to predict an average number of nucleobases within the sample genomic sequence.

CLAUSE 5. The method of any of clauses 1-4, further comprising generating the at least one predicted insert length by utilizing the insert length prediction model to predict a length of the sample genomic sequence and one or more adapter sequences appended to the sample genomic sequence.

CLAUSE 6. The method of any of clauses 1-5, further comprising determining the one or more cluster metrics by determining a signal intensity corresponding to the cluster of oligonucleotides.

CLAUSE 7. The method of any of clauses 1-6, further comprising:
identifying a set of candidate genomic regions within a reference genome; and
selecting, from among the set of candidate genomic regions, a candidate genomic region for mapping the first nucleotide read and the second nucleotide read instead of another candidate genomic region based on the at least one predicted insert length.

CLAUSE 8. The method of any of clauses 1-7, further comprising:
mapping the first nucleotide read and the second nucleotide read to the candidate genomic region comprising one or more of a structural variant, a variable number tandem repeat (VNTR), a short tandem repeat (STR), a segmental duplication, a long interspersed nucleotide element (LINE), or a short interspersed nucleotide element (SINE); and
determining the genotype call by determining the genotype call for the genomic coordinate within the structural variant, the VNTR, the STR, the segmental duplication, the LINE, or the SINE.

CLAUSE 9. The method of any of clauses 1-8, further comprising determine the one or more cluster metrics by determining one or more of:
a cluster intensity metric corresponding to the cluster of oligonucleotides;
a cluster gain metric indicating a difference between a first signal intensity emitted from the cluster of oligonucleotides in a luminescent state and a second light intensity emitted from the cluster of oligonucleotides in a non-luminescent state;
a cluster offset metric indicating a signal intensity corresponding to the cluster of oligonucleotides in a non-luminescent state;
a signal-to-noise ratio (SNR) differential metric indicating a difference between an SNR for the first nucleotide read and an SNR for the second nucleotide read;
a guanine-cytosine (GC) content metric indicating an amount of sequenced nucleotide bases within the first nucleotide read or the second nucleotide read that include a guanine base or a cytosine base;
a phasing metric indicating phasing or pre-phasing of oligonucleotides within the cluster of oligonucleotides;
a nucleobase content metric indicating amounts of sequenced nucleotide bases within the first nucleotide read or the second nucleotide read that are adenine, cytosine, guanine, or thymine bases;
a polyclonality metric indicating a probability that the cluster of oligonucleotides includes oligonucleotides from two or more genomic samples;
a homopolymer content metric indicating an amount of homopolymer content within the cluster of oligonucleotides;
a cluster size metric indicating a size of the cluster of oligonucleotides within a sequencing image;
a relative cluster offset metric indicating a difference in signal intensity emitted from the cluster of oligonucleotides compared to an average signal intensity for a sequencing well in which the cluster of oligonucleotides is located;
an overlap metric indicating a number of overlapping nucleobases in a shared sequence between the first nucleotide read and the second nucleotide read;
an SNR metric indicating a signal-to-noise ratio at one or more of a portion (e.g., an end) of the first nucleotide read or a portion (e.g., an end) of the second nucleotide read;
a base call quality metric indicating a quality of a nucleobase call at one or more of a portion (e.g., an end) of the first nucleotide read or a portion (e.g., an end) of the second nucleotide read;
a cluster position metric indicating a position of the cluster of oligonucleotides within a region of a nucleotide-sample slide;
a region position metric indicating a position of the region within the nucleotide-sample slide; or
a free energy metric indicating an amount of energy to fold a molecule to a lower energy state.

CLAUSE 10. The method of any of clauses 1-9, further comprising determine the one or more cluster metrics by determining a shape of the sample genomic sequence when within a chromosome.

CLAUSE 11. The method of any of clauses 1-10, further comprising determining the one or more cluster metrics by determining one or more overlapping nucleotide reads from different clusters of oligonucleotides that include nucleotide reads that map to a genomic region of the sample genomic sequence.

CLAUSE 12. The method of any of clauses 1-11, further comprising:
selecting training nucleotide reads with mapping quality scores that satisfy a threshold mapping quality score;
generating, using the insert length prediction model to process one or more training cluster metrics associated with the training nucleotide reads, predicted insert lengths of training sample genomic sequences corresponding to the training nucleotide reads; and
adjusting parameters of the insert length prediction model based on comparisons of ground-truth insert lengths of the training sample genomic sequences and the predicted insert lengths.

CLAUSE 13. The method of any of clauses 1-12, further comprising mapping the first nucleotide read and the second nucleotide read to a genomic region of a reference genome based on the at least one predicted insert length and nucleobase similarity between the genomic region and the first and second nucleotide reads.

CLAUSE 14. The method of any of clauses 1-13, further comprising generating the at least one predicted insert length by:
determining a length of a repeating sequence within a tandem repeat region of the sample genomic sequence; and
predicting a number of nucleobases for the at least one predicted insert length based on the length of the repeating sequence within the tandem repeat region.

CLAUSE 15. The method of any of clauses 1-14, further comprising determining the genotype call for the genomic coordinate by determining that the sample genomic sequence includes a structural variant based on comparing the at least one predicted insert length with an expected insert length for the sample genomic sequence.

CLAUSE 16. The method of any of clauses 1-15, further comprising determining the genotype call for the genomic coordinate by determining a tandem repeat corresponding to the genomic coordinate.

CLAUSE 17. The method of any of clauses 1-16, further comprising determining a repeat count of a repeat unit within a variable number tandem repeat (VNTR) by:
determining, for a set of haplotypes corresponding to the genomic coordinate, respective haplotype probabilities for the genomic sample at the genomic coordinate based on a set of nucleotide read pairs of the genomic sample corresponding to the genomic coordinate and a set of predicted insert lengths for the set of nucleotide read pairs;
selecting a highest haplotype probability from among the respective haplotype probabilities of the set of haplotypes; and
determining the repeat count of the repeat unit based on a haplotype length indicated by the highest haplotype probability.

CLAUSE 18. The method of any of clauses 1-17, further comprising determine at least a prior haplotype probability for each of the respective haplotype probabilities based on a nucleotide read pair and the set of predicted insert lengths by determining one or more of:
a first prior haplotype probability of the genomic sample comprising a first candidate haplotype of a first length with respect to a tandem repeat region based on a respective distribution of predicted insert lengths for the nucleotide read pair;
a second prior haplotype probability of the genomic sample comprising a second candidate haplotype of a second length with respect to the tandem repeat region based on the respective distribution of predicted insert lengths for the nucleotide read pair; or
a third prior haplotype probability of the genomic sample comprising a third candidate haplotype of a third length with respect to the tandem repeat region based on the respective distribution of predicted insert lengths for the nucleotide read pair.

CLAUSE 19. The method of any of clauses 1-18, further comprising determining at least a posterior haplotype probability for each of the respective haplotype probabilities based on the nucleotide read pair and the set of predicted insert lengths by determining one or more of:
a first posterior haplotype probability of the genomic sample comprising the first candidate haplotype of the first length with respect to the tandem repeat region based on the first prior haplotype probability and the respective distribution of predicted insert lengths for the nucleotide read pair;
a second posterior haplotype probability of the genomic sample comprising the second candidate haplotype of the second length with respect to the tandem repeat region based on the second prior haplotype probability and the respective distribution of predicted insert lengths for the nucleotide read pair; or
a third posterior haplotype probability of the genomic sample comprising the third candidate haplotype of the third length with respect to the tandem repeat region based on the third prior haplotype probability and the respective distribution of predicted insert lengths for the nucleotide read pair.

CLAUSE 20. The method of any of clauses 1-18, wherein:
the first candidate haplotype of the first length represents a spanning insert that spans the tandem repeat region;
the second candidate haplotype of the second length represents a flanking insert that flanks the tandem repeat region; and
the third candidate haplotype of the third length represents an internal insert that is within the tandem repeat region.

CLAUSE 21. The method of any of clauses 1-19, further comprising:
determining at least the prior haplotype probability for each of the respective haplotype probabilities by determining a set of prior haplotype probabilities comprising the first prior haplotype probability, the second prior haplotype probability, the third prior haplotype probability, and one or more additional prior haplotype probabilities of the genomic sample comprising one or more additional candidate haplotypes of one or more additional lengths with respect to the tandem repeat region; and
determining at least the posterior haplotype probability for each of the respective haplotype probabilities by determining a set of posterior haplotype probabilities comprising the first posterior haplotype probability, the second posterior haplotype probability, the third posterior haplotype probability, and one or more additional posterior haplotype probabilities of the genomic sample comprising the one or more additional candidate haplotypes of the one or more additional lengths with respect to the tandem repeat region.

CLAUSE 22. The method of any of clauses 1-21, further comprising determining the genotype call for the genomic coordinate by:
determining genotype probabilities based on one or more haplotype probabilities from the respective haplotype probabilities; and
selecting the genotype call corresponding to a highest genotype probability from among the genotype probabilities.

CLAUSE 23. The method of any of clauses 1-22, further comprising:
determining that the at least one predicted insert length differs by a threshold number of nucleobases from an expected insert length for the sample genomic sequence; and
selecting, based on the at least one predicted insert length differing by the threshold number of nucleobases from the expected insert length, the genomic coordinate as a candidate genomic coordinate for structural variant calling.

CLAUSE 24. The method of any of clauses 1-23, further comprising:
identifying, for the genomic coordinate, a set of candidate alleles comprising a first candidate allele corresponding to the at least one predicted insert length and a second candidate allele inconsistent with the at least one predicted insert length; and
determining the genotype call for the genomic coordinate by determining a structural variant call or other genotype call corresponding to the genomic coordinate based on a comparison of the first candidate allele and the second candidate allele.

CLAUSE 25. The method of any of clauses 1-24, further comprising:
identifying, for the genomic coordinate, a set of nucleotide read pairs of the genomic sample and a set of candidate alleles comprising a first candidate allele corresponding to the at least one predicted insert length and a second candidate allele inconsistent with the at least one predicted insert length;
determining, for the set of candidate alleles, nucleotide-read fragment probabilities reflecting likelihoods of nucleotide-read fragments from the set of nucleotide-read fragments supporting respective candidate alleles from among the set of candidate alleles;
identifying, from among the set of candidate alleles, the first candidate allele or the second candidate allele having a highest nucleotide-read fragment probability; and
determining the genotype call for the genomic coordinate by determining a structural variant call or other genotype call corresponding to the genomic coordinate based on the first candidate allele or the second candidate allele having the highest nucleotide-read fragment probability.

CLAUSE 26. A method comprising:
identifying, for clusters of oligonucleotides corresponding to sample genomic sequences for a genomic sample, nucleotide read pairs each comprising a first nucleotide read complementing a first portion of a respective sample genomic sequence and a second nucleotide read complementing a second portion of the respective sample genomic sequence;
determining one or more cluster metrics respectively associated with the clusters of oligonucleotides;
generating, using an insert length prediction model to process the one or more cluster metrics, predicted insert lengths of respective sample genomic sequences;
determining, based on the predicted insert lengths and the nucleotide read pairs, candidate allele probabilities for a set of candidate alleles corresponding to a genomic coordinate within the respective sample genomic sequences; and
determining a structural variant call for the genomic coordinate within the respective sample genomic sequences based on the candidate allele probabilities.

CLAUSE 27. The method of clause 26, wherein determining the structural variant call comprises determining a positive structural variant call identifying a presence of a structural variant or a negative structural variant call identifying an absence of the structural variant.

CLAUSE 28. The method of any of clauses 26-27, further comprising:
identifying, for the genomic coordinate, the set of candidate alleles comprising a first candidate allele corresponding to the predicted insert lengths and a second candidate allele inconsistent with the predicted insert lengths; and
determining the structural variant call for the genomic coordinate based on a comparison of the first candidate allele and the second candidate allele.

CLAUSE 29. The method of any of clauses 26-28, further comprising:
identifying, for the genomic coordinate and the nucleotide read pairs, the set of candidate alleles comprising a first candidate allele corresponding to a predicted insert length and a second candidate allele inconsistent with the predicted insert length;
determining, for the set of candidate alleles, nucleotide-read fragment probabilities reflecting likelihoods of nucleotide-read fragments from the nucleotide read pairs supporting respective candidate alleles from among the set of candidate alleles;
identifying, from among the set of candidate alleles, the first candidate allele or the second candidate allele having a highest nucleotide-read fragment probability; and
determining the structural variant call for the genomic coordinate based on the first candidate allele or the second candidate allele having the highest nucleotide-read fragment probability.

CLAUSE 30. The method of any of clauses 26-29, further comprising generating the predicted insert lengths by determining, using the insert length prediction model, one or more of distributions of predicted insert lengths of the sample genomic sequences or mean predicted insert lengths from the distributions of predicted insert lengths.

CLAUSE 31. The method of any of clauses 26-30, further comprising determining the distributions of predicted insert lengths by determining one or more of parametric distributions of predicted insert lengths, non-parametric distributions of predicted insert lengths, expectiles of predicted insert lengths, or quantiles of predicted insert lengths.

CLAUSE 32. The method of any of clauses 26-31, further comprising generating the predicted insert lengths by utilizing the insert length prediction model to predict an average number of nucleobases within the sample genomic sequences.

CLAUSE 33. The method of any of clauses 26-32, further comprising generating the predicted insert lengths by utilizing the insert length prediction model to predict lengths of the sample genomic sequences and one or more adapter sequences appended to the sample genomic sequences.

CLAUSE 34. The method of any of clauses 26-33, further comprising determining the one or more cluster metrics by determining a signal intensity corresponding to a cluster of oligonucleotides of the clusters of oligonucleotides.

CLAUSE 35. The method of any of clauses 26-34, further comprising:
identifying a set of candidate genomic regions within a reference genome; and
selecting, from among the set of candidate genomic regions, a candidate genomic region for mapping the first nucleotide read and the second nucleotide read instead of another candidate genomic region based on the predicted insert lengths.

CLAUSE 36. The method of any of clauses 26-35, wherein determining the one or more cluster metrics comprises determining, for a cluster of oligonucleotides of the clusters of oligonucleotides, one or more of:
a cluster intensity metric corresponding to the cluster of oligonucleotides;
a cluster gain metric indicating a difference between a first signal intensity emitted from the cluster of oligonucleotides in a luminescent state and a second light intensity emitted from the cluster of oligonucleotides in a non-luminescent state;
a cluster offset metric indicating a signal intensity corresponding to the cluster of oligonucleotides in a non-luminescent state;
a signal-to-noise ratio (SNR) differential metric indicating a difference between an SNR for the first nucleotide read and an SNR for the second nucleotide read;
a guanine-cytosine (GC) content metric indicating an amount of sequenced nucleotide bases within the first nucleotide read or the second nucleotide read that include a guanine base or a cytosine base;
a phasing metric indicating phasing or pre-phasing of oligonucleotides within the cluster of oligonucleotides;
a nucleobase content metric indicating amounts of sequenced nucleotide bases within the first nucleotide read or the second nucleotide read that are adenine, cytosine, guanine, or thymine bases;
a polyclonality metric indicating a probability that the cluster of oligonucleotides includes oligonucleotides from two or more genomic samples;
a homopolymer content metric indicating an amount of homopolymer content within the cluster of oligonucleotides;
a cluster size metric indicating a size of the cluster of oligonucleotides within a sequencing image;
a relative cluster offset metric indicating a difference in signal intensity emitted from the cluster of oligonucleotides compared to an average signal intensity for a sequencing well in which the cluster of oligonucleotides is located;
an overlap metric indicating a number of overlapping nucleobases in a shared sequence between the first nucleotide read and the second nucleotide read;
an SNR metric indicating a signal-to-noise ratio at one or more of a portion of the first nucleotide read or a portion of the second nucleotide read;
a base call quality metric indicating a quality of a nucleobase call at one or more of a portion of the first nucleotide read or a portion of the second nucleotide read;
a cluster position metric indicating a position of the cluster of oligonucleotides within a region of a nucleotide-sample slide;
a region position metric indicating a position of the region within the nucleotide-sample slide; or
a free energy metric indicating an amount of energy to fold a molecule to a lower energy state.

CLAUSE 37. The method of any of clauses 26-36, wherein determining the one or more cluster metrics comprises determining, for a cluster of oligonucleotides of the clusters of oligonucleotides, a shape of a sample genomic sequence when within a chromosome.

CLAUSE 38. The method of any of clauses 26-37, wherein determining the one or more cluster metrics comprises determining one or more overlapping nucleotide reads from different clusters of oligonucleotides that include nucleotide reads that map to a genomic region of the sample genomic sequence.

CLAUSE 39. The method of any of clauses 26-38, further comprising:
selecting training nucleotide reads with mapping quality scores that satisfy a threshold mapping quality score;
generating, using the insert length prediction model to process one or more training cluster metrics associated with the training nucleotide reads, predicted insert lengths of training sample genomic sequences corresponding to the training nucleotide reads; and
adjusting parameters of the insert length prediction model based on comparisons of ground-truth insert lengths of the training sample genomic sequences and the predicted insert lengths.

CLAUSE 40. The method of any of clauses 26-39, further comprising mapping the first nucleotide read and the second nucleotide read to a genomic region of a reference genome based on the predicted insert lengths and a nucleobase similarity between the genomic region and the first and second nucleotide reads.

CLAUSE 41. The method of any of clauses 26-40, further comprising generating a predicted insert length of the predicted insert lengths by:
determining lengths of repeating sequences within a tandem repeat region of the sample genomic sequences; and
predicting a number of nucleobases for the predicted insert length based on the lengths of the repeating sequences within the tandem repeat region.

CLAUSE 42. The method of any of clauses 26-41, further comprising determining the structural variant call based on comparing the predicted insert lengths with an expected insert length for the sample genomic sequences.

CLAUSE 43. The method of any of clauses 26-42, further comprising:
determining that at least one of the predicted insert lengths differs by a threshold number of nucleobases from an expected insert length for the sample genomic sequences; and
selecting, based on the at least one of the predicted insert lengths differing by the threshold number of nucleobases from the expected insert length, the genomic coordinate as a candidate genomic coordinate for structural variant calling.

CLAUSE 44. A method comprising:
identifying, for clusters of oligonucleotides corresponding to sample genomic sequences for a genomic sample, a set of nucleotide read pairs each comprising a first nucleotide read complementing a first portion of a respective sample genomic sequence and a second nucleotide read complementing a second portion of the respective sample genomic sequence;
determining one or more cluster metrics respectively associated with the clusters of oligonucleotides;
generating, using an insert length prediction model to process the one or more cluster metrics, a set of predicted insert lengths of respective sample genomic sequences;
determining, based on the set of predicted insert lengths and the set of nucleotide read pairs, a set of candidate haplotype probabilities for a set of candidate haplotypes corresponding to a genomic coordinate within the respective sample genomic sequences; and
determining a tandem repeat call for the genomic coordinate within the respective sample genomic sequences based on the set of candidate haplotype probabilities.

CLAUSE 45. The method of clause 44, wherein determining the tandem repeat call comprises determining a repeat count of a repeat unit of a variable number tandem repeat (VNTR) or a short tandem repeat (STR) corresponding to the genomic coordinate.

CLAUSE 46. The method of any of clauses 44-45, wherein determining the tandem repeat call comprises determining a repeat count of a repeat unit within a variable number tandem repeat (VNTR) by:
determining the set of candidate haplotype probabilities by determining, for the set of candidate haplotypes corresponding to the genomic coordinate, respective haplotype probabilities for the genomic sample at the genomic coordinate based the set of nucleotide read pairs of the genomic sample corresponding to the genomic coordinate and the set of predicted insert lengths for the set of nucleotide read pairs;
selecting a highest haplotype probability from among the respective haplotype probabilities of the set of candidate haplotypes; and
determining the repeat count of the repeat unit based on a haplotype length indicated by the highest haplotype probability.

CLAUSE 47. The method of any of clauses 44-46, further comprising determining at least a prior haplotype probability for each of the respective haplotype probabilities based on a nucleotide read pair and the set of predicted insert lengths by determining one or more of:
a first prior haplotype probability of the genomic sample comprising a first candidate haplotype of a first length with respect to a tandem repeat region based on a respective distribution of predicted insert lengths for the nucleotide read pair;
a second prior haplotype probability of the genomic sample comprising a second candidate haplotype of a second length with respect to the tandem repeat region based on the respective distribution of predicted insert lengths for the nucleotide read pair; or
a third prior haplotype probability of the genomic sample comprising a third candidate haplotype of a third length with respect to the tandem repeat region based on the respective distribution of predicted insert lengths for the nucleotide read pair.

CLAUSE 48. The method of any of clauses 44-47, further comprising determining at least a posterior haplotype probability for each of the respective haplotype probabilities based on the set of nucleotide read pairs and the set of predicted insert lengths by determining one or more of:
a first posterior haplotype probability of the genomic sample comprising the first candidate haplotype of the first length with respect to the tandem repeat region based on the first prior haplotype probability and the respective distribution of predicted insert lengths for the set of nucleotide read pairs;
a second posterior haplotype probability of the genomic sample comprising the second candidate haplotype of the second length with respect to the tandem repeat region based on the second prior haplotype probability and the respective distribution of predicted insert lengths for the set of nucleotide read pairs; or
a third posterior haplotype probability of the genomic sample comprising the third candidate haplotype of the third length with respect to the tandem repeat region based on the third prior haplotype probability and the respective distribution of predicted insert lengths for the set of nucleotide read pairs.

CLAUSE 49. The method of any of clauses 44-48, wherein:
the first candidate haplotype of the first length represents a spanning insert that spans the tandem repeat region;
the second candidate haplotype of the second length represents a flanking insert that flanks the tandem repeat region; and
the third candidate haplotype of the third length represents an internal insert that is within the tandem repeat region.

CLAUSE 50. The method of any of clauses 44-49, further comprising:
determining at least the prior haplotype probability for each of the respective haplotype probabilities by determining a set of prior haplotype probabilities comprising the first prior haplotype probability, the second prior haplotype probability, the third prior haplotype probability, and one or more additional prior haplotype probabilities of the genomic sample comprising one or more additional candidate haplotypes of one or more additional lengths with respect to the tandem repeat region; and
determining at least the posterior haplotype probability for each of the respective haplotype probabilities by determining a set of posterior haplotype probabilities comprising the first posterior haplotype probability, the second posterior haplotype probability, the third posterior haplotype probability, and one or more additional posterior haplotype probabilities of the genomic sample comprising the one or more additional candidate haplotypes of the one or more additional lengths with respect to the tandem repeat region

CLAUSE 51. The method of any of clauses 44-50, wherein generating the set of predicted insert lengths comprises determining, using the insert length prediction model, one or more of distributions of predicted insert lengths of the sample genomic sequences or mean predicted insert lengths from the distributions of predicted insert lengths.

CLAUSE 52. The method of any of clauses 44-51, wherein determining the distributions of the set of predicted insert lengths comprises determining one or more of parametric distributions of predicted insert lengths, non-parametric distributions of predicted insert lengths, expectiles of predicted insert lengths, or quantiles of predicted insert lengths.

CLAUSE 53. The method of any of clauses 44-52, wherein generating the set of predicted insert lengths comprises utilizing the insert length prediction model to predict an average number of nucleobases within the sample genomic sequences.

CLAUSE 54. The method of any of clauses 44-53, wherein generating the set of predicted insert lengths comprises utilizing the insert length prediction model to predict lengths of the sample genomic sequences and one or more adapter sequences appended to the sample genomic sequences.

CLAUSE 55. The method of any of clauses 44-54, wherein determining the one or more cluster metrics comprises determining a signal intensity corresponding to the clusters of oligonucleotides.

CLAUSE 56. The method of any of clauses 44-55, further comprising:
identifying a set of candidate genomic regions within a reference genome; and
selecting, from among the set of candidate genomic regions, a candidate genomic region for mapping the first nucleotide read and the second nucleotide read instead of another candidate genomic region based on the set of predicted insert lengths.

CLAUSE 57. The method of any of clauses 44-56, wherein determining the one or more cluster metrics comprises determining, for a cluster of oligonucleotides of the clusters of oligonucleotides, one or more of:
a cluster intensity metric corresponding to the cluster of oligonucleotides;
a cluster gain metric indicating a difference between a first signal intensity emitted from the cluster of oligonucleotides in a luminescent state and a second light intensity emitted from the cluster of oligonucleotides in a non-luminescent state;
a cluster offset metric indicating a signal intensity corresponding to the cluster of oligonucleotides in a non-luminescent state;
a signal-to-noise ratio (SNR) differential metric indicating a difference between an SNR for the first nucleotide read and an SNR for the second nucleotide read;
a guanine-cytosine (GC) content metric indicating an amount of sequenced nucleotide bases within the first nucleotide read or the second nucleotide read that include a guanine base or a cytosine base;
a phasing metric indicating phasing or pre-phasing of oligonucleotides within the cluster of oligonucleotides;
a nucleobase content metric indicating amounts of sequenced nucleotide bases within the first nucleotide read or the second nucleotide read that are adenine, cytosine, guanine, or thymine bases;
a polyclonality metric indicating a probability that the cluster of oligonucleotides includes oligonucleotides from two or more genomic samples;
a homopolymer content metric indicating an amount of homopolymer content within the cluster of oligonucleotides;
a cluster size metric indicating a size of the cluster of oligonucleotides within a sequencing image;
a relative cluster offset metric indicating a difference in signal intensity emitted from the cluster of oligonucleotides compared to an average signal intensity for a sequencing well in which the cluster of oligonucleotides is located;
an overlap metric indicating a number of overlapping nucleobases in a shared sequence between the first nucleotide read and the second nucleotide read;
an SNR metric indicating a signal-to-noise ratio at one or more of a portion of the first nucleotide read or a portion of the second nucleotide read;
a base call quality metric indicating a quality of a nucleobase call at one or more of a portion of the first nucleotide read or a portion of the second nucleotide read;
a cluster position metric indicating a position of the cluster of oligonucleotides within a region of a nucleotide-sample slide;
a region position metric indicating a position of the region within the nucleotide-sample slide; or
a free energy metric indicating an amount of energy to fold a molecule to a lower energy state.

CLAUSE 58. The method of any of clauses 44-57, wherein determining the one or more cluster metrics comprises determining shapes of the sample genomic sequences when within a chromosome.

CLAUSE 59. The method of any of clauses 44-58, wherein determining the one or more cluster metrics comprises determining one or more overlapping nucleotide reads from different clusters of oligonucleotides that include nucleotide reads that map to a genomic region of the sample genomic sequences.

CLAUSE 60. The method of any of clauses 44-59, further comprising mapping the first nucleotide read and the second nucleotide read to a genomic region of a reference genome based on the set of predicted insert lengths and a nucleobase similarity between the genomic region and the first and second nucleotide reads.

CLAUSE 61. The method of any of clauses 44-60, wherein generating the set of predicted insert lengths comprises:
determining lengths of repeating sequences within a tandem repeat region of the sample genomic sequences; and
predicting a number of nucleobases for at least one predicted insert length based on the length of the repeating sequence within the tandem repeat region.

The methods described herein can be used in conjunction with a variety of nucleic acid sequencing techniques. Particularly applicable techniques are those wherein nucleic acids are attached at fixed locations in an array such that their relative positions do not change and wherein the array is repeatedly imaged. Embodiments in which images are obtained in different color channels, for example, coinciding with different labels used to distinguish one nucleotide base type from another are particularly applicable. In some embodiments, the process to determine the nucleotide sequence of a target nucleic acid (i.e., a nucleic acid polymer) can be an automated process. Preferred embodiments include sequencing-by-synthesis (SBS) techniques.

SBS techniques generally involve the enzymatic extension of a nascent nucleic acid strand through the iterative addition of nucleotides against a template strand. In traditional methods of SBS, a single nucleotide monomer may be provided to a target nucleotide in the presence of a polymerase in each delivery. However, in the methods described herein, more than one type of nucleotide monomer can be provided to a target nucleic acid in the presence of a polymerase in a delivery.

SBS can utilize nucleotide monomers that have a terminator moiety or those that lack any terminator moieties. Methods utilizing nucleotide monomers lacking terminators include, for example, pyrosequencing and sequencing using y-phosphate-labeled nucleotides, as set forth in further detail below. In methods using nucleotide monomers lacking terminators, the number of nucleotides added in each cycle is generally variable and dependent upon the template sequence and the mode of nucleotide delivery. For SBS techniques that utilize nucleotide monomers having a terminator moiety, the terminator can be effectively irreversible under the sequencing conditions used as is the case for traditional Sanger sequencing which utilizes dideoxynucleotides, or the terminator can be reversible as is the case for sequencing methods developed by Solexa (now Illumina, Inc.).

SBS techniques can utilize nucleotide monomers that have a label moiety or those that lack a label moiety. Accordingly, incorporation events can be detected based on a characteristic of the label, such as fluorescence of the label; a characteristic of the nucleotide monomer such as molecular weight or charge; a byproduct of incorporation of the nucleotide, such as release of pyrophosphate; or the like. In embodiments, where two or more different nucleotides are present in a sequencing reagent, the different nucleotides can be distinguishable from each other, or alternatively, the two or more different labels can be the indistinguishable under the detection techniques being used. For example, the different nucleotides present in a sequencing reagent can have different labels and they can be distinguished using appropriate optics as exemplified by the sequencing methods developed by Solexa (now Illumina, Inc.).

Preferred embodiments include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996), “Real-time DNA sequencing using detection of pyrophosphate release,” Analytical Biochemistry 242(1), 84-9; Ronaghi, M. (2001), “Pyrosequencing sheds light on DNA sequencing,” Genome Res. 11(1), 3-11; Ronaghi, M., Uhlen, M. and Nyren, P. (1998), “A sequencing method based on real-time pyrophosphate.” Science 281(5375), 363; U.S. Pat. Nos. 6,210,891; 6,258,568 and 6,274,320, the disclosures of which are incorporated herein by reference in their entireties). In pyrosequencing, released Ppi can be detected by being immediately converted to adenosine triphosphate (ATP) by ATP sulfurylase, and the level of ATP generated is detected via luciferase-produced photons. The nucleic acids to be sequenced can be attached to features in an array and the array can be imaged to capture the chemiluminescent signals that are produced due to incorporation of a nucleotides at the features of the array. An image can be obtained after the array is treated with a particular nucleotide type (e.g., A, T, C or G). Images obtained after addition of each nucleotide type will differ with regard to which features in the array are detected. These differences in the image reflect the different sequence content of the features on the array. However, the relative locations of each feature will remain unchanged in the images. The images can be stored, processed and analyzed using the methods set forth herein. For example, images obtained after treatment of the array with each different nucleotide type can be handled in the same way as exemplified herein for images obtained from different detection channels for reversible terminator-based sequencing methods.

In another exemplary type of SBS, cycle sequencing is accomplished by stepwise addition of reversible terminator nucleotides containing, for example, a cleavable or photobleachable dye label as described, for example, in WO 04/018497 and U.S. Pat. No. 7,057,026, the disclosures of which are incorporated herein by reference. This approach is being commercialized by Solexa (now Illumina Inc.), and is also described in WO 91/06678 and WO 07/123,744, each of which is incorporated herein by reference. The availability of fluorescently-labeled terminators in which both the termination can be reversed and the fluorescent label cleaved facilitates efficient cyclic reversible termination (CRT) sequencing. Polymerases can also be co-engineered to efficiently incorporate and extend from these modified nucleotides.

Preferably in reversible terminator-based sequencing embodiments, the labels do not substantially inhibit extension under SBS reaction conditions. However, the detection labels can be removable, for example, by cleavage or degradation. Images can be captured following incorporation of labels into arrayed nucleic acid features. In particular embodiments, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially and an image of the array can be obtained between each addition step. In such embodiments, each image will show nucleic acid features that have incorporated nucleotides of a particular type. Different features are present or absent in the different images due the different sequence content of each feature. However, the relative position of the features will remain unchanged in the images. Images obtained from such reversible terminator-SBS methods can be stored, processed and analyzed as set forth herein. Following the image capture step, labels can be removed and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles. Examples of useful labels and removal methods are set forth below.

In particular embodiments some or all of the nucleotide monomers can include reversible terminators. In such embodiments, reversible terminators/cleavable fluors can include fluor linked to the ribose moiety via a 3′ ester linkage (Metzker, Genome Res. 15:1767-1776 (2005), which is incorporated herein by reference). Other approaches have separated the terminator chemistry from the cleavage of the fluorescence label (Ruparel et al., Proc Natl Acad Sci USA 102: 5932-7 (2005), which is incorporated herein by reference in its entirety). Ruparel et al described the development of reversible terminators that used a small 3′ allyl group to block extension, but could easily be deblocked by a short treatment with a palladium catalyst. The fluorophore was attached to the base via a photocleavable linker that could easily be cleaved by a 30 second exposure to long wavelength UV light. Thus, either disulfide reduction or photocleavage can be used as a cleavable linker. Another approach to reversible termination is the use of natural termination that ensues after placement of a bulky dye on a dNTP. The presence of a charged bulky dye on the dNTP can act as an effective terminator through steric and/or electrostatic hindrance. The presence of one incorporation event prevents further incorporations unless the dye is removed. Cleavage of the dye removes the fluor and effectively reverses the termination. Examples of modified nucleotides are also described in U.S. Pat. Nos. 7,427,673, and 7,057,026, the disclosures of which are incorporated herein by reference in their entireties.

Additional exemplary SBS systems and methods which can be utilized with the methods and systems described herein are described in U.S. Patent Application Publication No. 2007/0166705, U.S. Patent Application Publication No. 2006/0188901, U.S. Pat. No. 7,057,026, U.S. Patent Application Publication No. 2006/0240439, U.S. Patent Application Publication No. 2006/0281109, PCT Publication No. WO 05/065814, U.S. Patent Application Publication No. 2005/0100900, PCT Publication No. WO 06/064199, PCT Publication No. WO 07/010,251, U.S. Patent Application Publication No. 2012/0270305 and U.S. Patent Application Publication No. 2013/0260372, the disclosures of which are incorporated herein by reference in their entireties.

Some embodiments can utilize detection of four different nucleotides using fewer than four different labels. For example, SBS can be performed utilizing methods and systems described in the incorporated materials of U.S. Patent Application Publication No. 2013/0079232. As a first example, a pair of nucleotide types can be detected at the same wavelength, but distinguished based on a difference in intensity for one member of the pair compared to the other, or based on a change to one member of the pair (e.g. via chemical modification, photochemical modification or physical modification) that causes apparent signal to appear or disappear compared to the signal detected for the other member of the pair. As a second example, three of four different nucleotide types can be detected under particular conditions while a fourth nucleotide type lacks a label that is detectable under those conditions, or is minimally detected under those conditions (e.g., minimal detection due to background fluorescence, etc.). Incorporation of the first three nucleotide types into a nucleic acid can be determined based on presence of their respective signals and incorporation of the fourth nucleotide type into the nucleic acid can be determined based on absence or minimal detection of any signal. As a third example, one nucleotide type can include label(s) that are detected in two different channels, whereas other nucleotide types are detected in no more than one of the channels. The aforementioned three exemplary configurations are not considered mutually exclusive and can be used in various combinations. An exemplary embodiment that combines all three examples, is a fluorescent-based SBS method that uses a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength) and a fourth nucleotide type that lacks a label that is not, or minimally, detected in either channel (e.g. dGTP having no label).

Further, as described in the incorporated materials of U.S. Patent Application Publication No. 2013/0079232, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

Some embodiments can utilize sequencing by ligation techniques. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features are present or absent in the different images due the different sequence content of each feature, but the relative position of the features will remain unchanged in the images. Images obtained from ligation-based sequencing methods can be stored, processed and analyzed as set forth herein. Exemplary SBS systems and methods which can be utilized with the methods and systems described herein are described in U.S. Pat. Nos. 6,969,488, 6,172,218, and 6,306,597, the disclosures of which are incorporated herein by reference in their entireties.

Some embodiments can utilize nanopore sequencing (Deamer, D. W. & Akeson, M. “Nanopores and nucleic acids: prospects for ultrarapid sequencing,” Trends Biotechnol. 18, 147-151 (2000); Deamer, D. and D. Branton, “Characterization of nucleic acids by nanopore analysis.” Acc. Chem. Res. 35:817-825 (2002); Li, J., M. Gershow, D. Stein, E. Brandin, and J. A. Golovchenko, “DNA molecules and configurations in a solid-state nanopore microscope,” Nat. Mater. 2:611-615 (2003), the disclosures of which are incorporated herein by reference in their entireties). In such embodiments, the target nucleic acid passes through a nanopore. The nanopore can be a synthetic pore or biological membrane protein, such as a-hemolysin. As the target nucleic acid passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores,” Clin. Chem. 53, 1996-2001 (2007); Healy, K., “Nanopore-based single-molecule DNA analysis,” Nanomed. 2, 459-481 (2007); Cockroft, S. L., Chu, J., Amorin, M. & Ghadiri, M. R., “A single-molecule nanopore device detects DNA polymerase activity with single-nucleotide resolution,” J. Am. Chem. Soc. 130, 818-820 (2008), the disclosures of which are incorporated herein by reference in their entireties). Data obtained from nanopore sequencing can be stored, processed and analyzed as set forth herein. In particular, the data can be treated as an image in accordance with the exemplary treatment of optical images and other images that is set forth herein.

Some embodiments can utilize methods involving the real-time monitoring of DNA polymerase activity. Nucleotide incorporations can be detected through fluorescence resonance energy transfer (FRET) interactions between a fluorophore-bearing polymerase and y-phosphate-labeled nucleotides as described, for example, in U.S. Pat. Nos. 7,329,492 and 7,211,414 (each of which is incorporated herein by reference) or nucleotide incorporations can be detected with zero-mode waveguides as described, for example, in U.S. Pat. No. 7,315,019 (which is incorporated herein by reference) and using fluorescent nucleotide analogs and engineered polymerases as described, for example, in U.S. Pat. No. 7,405,281 and U.S. Patent Application Publication No. 2008/0108082 (each of which is incorporated herein by reference). The illumination can be restricted to a zeptoliter-scale volume around a surface-tethered polymerase such that incorporation of fluorescently labeled nucleotides can be observed with low background (Levene, M. J. et al., “Zero-mode waveguides for single-molecule analysis at high concentrations,” Science 299, 682-686 (2003); Lundquist, P. M. et al., “Parallel confocal detection of single molecules in real time,” Opt. Lett. 33, 1026-1028 (2008); Korlach, J. et al., “Selective aluminum passivation for targeted immobilization of single DNA polymerase molecules in zero-mode waveguide nano structures,” Proc. Natl. Acad. Sci. USA 105, 1176-1181 (2008), the disclosures of which are incorporated herein by reference in their entireties). Images obtained from such methods can be stored, processed and analyzed as set forth herein.

Some SBS embodiments include detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in US 2009/0026082 A1; US 2009/0127589 A1; US 2010/0137143 A1; or US 2010/0282617 A1, each of which is incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

The above SBS methods can be advantageously carried out in multiplex formats such that multiple different target nucleic acids are manipulated simultaneously. In particular embodiments, different target nucleic acids can be treated in a common reaction vessel or on a surface of a particular substrate. This allows convenient delivery of sequencing reagents, removal of unreacted reagents and detection of incorporation events in a multiplex manner. In embodiments using surface-bound target nucleic acids, the target nucleic acids can be in an array format. In an array format, the target nucleic acids can be typically bound to a surface in a spatially distinguishable manner. The target nucleic acids can be bound by direct covalent attachment, attachment to a bead or other particle or binding to a polymerase or other molecule that is attached to the surface. The array can include a single copy of a target nucleic acid at each site (also referred to as a feature) or multiple copies having the same sequence can be present at each site or feature. Multiple copies can be produced by amplification methods such as, bridge amplification or emulsion PCR as described in further detail below.

The methods set forth herein can use arrays having features at any of a variety of densities including, for example, at least about 10 features/cm2, 100 features/cm2, 500 features/cm2, 1,000 features/cm2, 5,000 features/cm2, 10,000 features/cm2, 50,000 features/cm2, 100,000 features/cm2, 1,000,000 features/cm2, 5,000,000 features/cm2, or higher.

An advantage of the methods set forth herein is that they provide for rapid and efficient detection of a plurality of target nucleic acid in parallel. Accordingly the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more immobilized DNA fragments, the system comprising components such as pumps, valves, reservoirs, fluidic lines and the like. A flow cell can be configured and/or used in an integrated system for detection of target nucleic acids. Exemplary flow cells are described, for example, in US 2010/0111768 A1 and U.S. Ser. No. 13/273,666, each of which is incorporated herein by reference. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeq™ platform (Illumina, Inc., San Diego, CA) and devices described in U.S. Ser. No. 13/273,666, which is incorporated herein by reference.

The sequencing system described above sequences nucleic acid polymers present in samples received by a sequencing device. As defined herein, “sample” and its derivatives, is used in its broadest sense and includes any specimen, culture and the like that is suspected of including a target. In some embodiments, the sample comprises DNA, RNA, PNA, LNA, chimeric or hybrid forms of nucleic acids. The sample can include any biological, clinical, surgical, agricultural, atmospheric or aquatic-based specimen containing one or more nucleic acids. The term also includes any isolated nucleic acid sample such a genomic DNA, fresh-frozen or formalin-fixed paraffin-embedded nucleic acid specimen. It is also envisioned that the sample can be from a single individual, a collection of nucleic acid samples from genetically related members, nucleic acid samples from genetically unrelated members, nucleic acid samples (matched) from a single individual such as a tumor sample and normal tissue sample, or sample from a single source that contains two distinct forms of genetic material such as maternal and fetal DNA obtained from a maternal subject, or the presence of contaminating bacterial DNA in a sample that contains plant or animal DNA. In some embodiments, the source of nucleic acid material can include nucleic acids obtained from a newborn, for example as typically used for newborn screening.

The nucleic acid sample can include high molecular weight material such as genomic DNA (gDNA). The sample can include low molecular weight material such as nucleic acid molecules obtained from FFPE or archived DNA samples. In another embodiment, low molecular weight material includes enzymatically or mechanically fragmented DNA. The sample can include cell-free circulating DNA. In some embodiments, the sample can include nucleic acid molecules obtained from biopsies, tumors, scrapings, swabs, blood, mucus, urine, plasma, semen, hair, laser capture micro-dissections, surgical resections, and other clinical or laboratory obtained samples. In some embodiments, the sample can be an epidemiological, agricultural, forensic or pathogenic sample. In some embodiments, the sample can include nucleic acid molecules obtained from an animal such as a human or mammalian source. In another embodiment, the sample can include nucleic acid molecules obtained from a non-mammalian source such as a plant, bacteria, virus or fungus. In some embodiments, the source of the nucleic acid molecules may be an archived or extinct sample or species.

Further, the methods and compositions disclosed herein may be useful to amplify a nucleic acid sample having low-quality nucleic acid molecules, such as degraded and/or fragmented genomic DNA from a forensic sample. In one embodiment, forensic samples can include nucleic acids obtained from a crime scene, nucleic acids obtained from a missing persons DNA database, nucleic acids obtained from a laboratory associated with a forensic investigation or include forensic samples obtained by law enforcement agencies, one or more military services or any such personnel. The nucleic acid sample may be a purified sample or a crude DNA containing lysate, for example derived from a buccal swab, paper, fabric or other substrate that may be impregnated with saliva, blood, or other bodily fluids. As such, in some embodiments, the nucleic acid sample may comprise low amounts of, or fragmented portions of DNA, such as genomic DNA. In some embodiments, target sequences can be present in one or more bodily fluids including but not limited to, blood, sputum, plasma, semen, urine and serum. In some embodiments, target sequences can be obtained from hair, skin, tissue samples, autopsy or remains of a victim. In some embodiments, nucleic acids including one or more target sequences can be obtained from a deceased animal or human. In some embodiments, target sequences can include nucleic acids obtained from non-human DNA such a microbial, plant or entomological DNA. In some embodiments, target sequences or amplified target sequences are directed to purposes of human identification. In some embodiments, the disclosure relates generally to methods for identifying characteristics of a forensic sample. In some embodiments, the disclosure relates generally to human identification methods using one or more target specific primers disclosed herein or one or more target specific primers designed using the primer design criteria outlined herein. In one embodiment, a forensic or human identification sample containing at least one target sequence can be amplified using any one or more of the target-specific primers disclosed herein or using the primer criteria outlined herein.

The components of the insert length prediction system 106 can include software, hardware, or both. For example, the components of the insert length prediction system 106 can include one or more instructions stored on a computer-readable storage medium and executable by processors of one or more computing devices (e.g., the client device 108). When executed by the one or more processors, the computer-executable instructions of the insert length prediction system 106 can cause the computing devices to perform the bubble detection methods described herein. Alternatively, the components of the insert length prediction system 106 can comprise hardware, such as special purpose processing devices to perform a certain function or group of functions. Additionally, or alternatively, the components of the insert length prediction system 106 can include a combination of computer-executable instructions and hardware.

Furthermore, the components of the insert length prediction system 106 performing the functions described herein with respect to the insert length prediction system 106 may, for example, be implemented as part of a stand-alone application, as a module of an application, as a plug-in for applications, as a library function or functions that may be called by other applications, and/or as a cloud-computing model. Thus, components of the insert length prediction system 106 may be implemented as part of a stand-alone application on a personal computing device or a mobile device. Additionally, or alternatively, the components of the insert length prediction system 106 may be implemented in any application that provides sequencing services including, but not limited to Illumina BaseSpace, Illumina DRAGEN, or Illumina TruSight software. “Illumina,” “BaseSpace,” “DRAGEN,” and “TruSight,” are either registered trademarks or trademarks of Illumina, Inc. in the United States and/or other countries.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., a memory, etc.), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (SSDs) (e.g., based on RAM), Flash memory, phase-change memory (PCM), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a NIC), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed at a processor, cause a general purpose computer, special purpose computer, or special purpose processing device to perform a certain function or group of functions. In some embodiments, computer-executable instructions are executed on a general-purpose computer to turn the general-purpose computer into a special purpose computer implementing elements of the disclosure. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. In this description, “cloud computing” is defined as a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (SaaS), Platform as a Service (PaaS), and Infrastructure as a Service (IaaS). A cloud-computing model can also be deployed using different deployment models such as private cloud, community cloud, public cloud, hybrid cloud, and so forth. In this description and in the claims, a “cloud-computing environment” is an environment in which cloud computing is employed.

FIG. 14 illustrates a block diagram of a computing device 1400 (e.g., the client device 108, the server device(s) 102, and/or the server device(s) 102) that may be configured to perform one or more of the processes described above. One will appreciate that one or more computing devices such as the computing device 1400 may implement the insert length prediction system 106 and the sequencing system 104. As shown by FIG. 14, the computing device 1400 can comprise a processor 1402, a memory 1404, a storage device 1406, an I/O interface 1408, and a communication interface 1410, which may be communicatively coupled by way of a communication infrastructure 1412. In certain embodiments, the computing device 1400 can include fewer or more components than those shown in FIG. 14. The following paragraphs describe components of the computing device 1400 shown in FIG. 14 in additional detail.

In one or more embodiments, the processor 1402 includes hardware for executing instructions, such as those making up a computer program. As an example, and not by way of limitation, to execute instructions for dynamically modifying workflows, the processor 1402 may retrieve (or fetch) the instructions from an internal register, an internal cache, the memory 1404, or the storage device 1406 and decode and execute them. The memory 1404 may be a volatile or non-volatile memory used for storing data, metadata, and programs for execution by the processor(s). The storage device 1406 includes storage, such as a hard disk, flash disk drive, or other digital storage device, for storing data or instructions for performing the methods described herein.

The I/O interface 1408 allows a user to provide input to, receive output from, and otherwise transfer data to and receive data from computing device 1400. The I/O interface 1408 may include a mouse, a keypad or a keyboard, a touch screen, a camera, an optical scanner, network interface, modem, other known I/O devices or a combination of such I/O interfaces. The I/O interface 1408 may include one or more devices for presenting output to a user, including, but not limited to, a graphics engine, a display (e.g., a display screen), one or more output drivers (e.g., display drivers), one or more audio speakers, and one or more audio drivers. In certain embodiments, the I/O interface 1408 is configured to provide graphical data to a display for presentation to a user. The graphical data may be representative of one or more graphical user interfaces and/or any other graphical content as may serve a particular implementation.

The communication interface 1410 can include hardware, software, or both. In any event, the communication interface 1410 can provide one or more interfaces for communication (such as, for example, packet-based communication) between the computing device 1400 and one or more other computing devices or networks. As an example, and not by way of limitation, the communication interface 1410 may include a network interface controller (NIC) or network adapter for communicating with an Ethernet or other wire-based network or a wireless NIC (WNIC) or wireless adapter for communicating with a wireless network, such as a WI-Fl.

Additionally, the communication interface 1410 may facilitate communications with various types of wired or wireless networks. The communication interface 1410 may also facilitate communications using various communication protocols. The communication infrastructure 1412 may also include hardware, software, or both that couples components of the computing device 1400 to each other. For example, the communication interface 1410 may use one or more networks and/or protocols to enable a plurality of computing devices connected by a particular infrastructure to communicate with each other to perform one or more aspects of the processes described herein. To illustrate, the sequencing process can allow a plurality of devices (e.g., a client device, sequencing device, and server device(s)) to exchange information such as sequencing data and error notifications.

In the foregoing specification, the present disclosure has been described with reference to specific exemplary embodiments thereof. Various embodiments and aspects of the present disclosure(s) are described with reference to details discussed herein, and the accompanying drawings illustrate the various embodiments. The description above and drawings are illustrative of the disclosure and are not to be construed as limiting the disclosure. Numerous specific details are described to provide a thorough understanding of various embodiments of the present disclosure.

The present disclosure may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel with one another or in parallel with different instances of the same or similar steps/acts. The scope of the present application is, therefore, indicated by the appended claims rather than by the foregoing description. All changes that come within the meaning and range of equivalency of the claims are to be embraced within their scope.

What is claimed is:

1. A system comprising:
at least one processor; and
a non-transitory computer readable medium comprising instructions that, when executed by the at least one processor, cause the system to:
identify, for a cluster of oligonucleotides corresponding to a sample genomic sequence for a genomic sample, a nucleotide read pair comprising a first nucleotide read complementing a first portion of the sample genomic sequence and a second nucleotide read complementing a second portion of the sample genomic sequence;
determine one or more cluster metrics associated with the cluster of oligonucleotides;
generate, using an insert length prediction model to process the one or more cluster metrics, at least one predicted insert length of the sample genomic sequence; and
determine a genotype call for a genomic coordinate within the sample genomic sequence based on the at least one predicted insert length.

2. The system of claim 1, further comprising instructions that, when executed by the at least one processor, cause the system to generate the at least one predicted insert length by determining, using the insert length prediction model, one or more of a distribution of predicted insert lengths of the sample genomic sequence or a mean predicted insert length from the distribution of predicted insert lengths.

3. The system of claim 2, further comprising instructions that, when executed by the at least one processor, cause the system to determine the distribution of predicted insert lengths by determining one or more of a parametric distribution of predicted insert lengths, a non-parametric distribution of predicted insert lengths, an expectile of predicted insert lengths, or a quantile of predicted insert lengths.

4. The system of claim 1, further comprising instructions that, when executed by the at least one processor, cause the system to generate the at least one predicted insert length by utilizing the insert length prediction model to predict an average number of nucleobases within the sample genomic sequence.

5. The system of claim 1, further comprising instructions that, when executed by the at least one processor, cause the system to generate the at least one predicted insert length by utilizing the insert length prediction model to predict a length of the sample genomic sequence and one or more adapter sequences appended to the sample genomic sequence.

6. The system of claim 1, further comprising instructions that, when executed by the at least one processor, cause the system to determine the one or more cluster metrics by determining a signal intensity corresponding to the cluster of oligonucleotides.

7. The system of claim 1, further comprising instructions that, when executed by the at least one processor, cause the system to:
identify a set of candidate genomic regions within a reference genome; and
select, from among the set of candidate genomic regions, a candidate genomic region for mapping the first nucleotide read and the second nucleotide read instead of another candidate genomic region based on the at least one predicted insert length.

8. The system of claim 7, further comprising instructions that, when executed by the at least one processor, cause the system to:
map the first nucleotide read and the second nucleotide read to the candidate genomic region comprising one or more of a structural variant, a variable number tandem repeat (VNTR), a short tandem repeat (STR), a segmental duplication, a long interspersed nucleotide element (LINE), or a short interspersed nucleotide element (SINE); and
determine the genotype call by determining the genotype call for the genomic coordinate within the structural variant, the VNTR, the STR, the segmental duplication, the LINE, or the SINE.

9. The system of claim 1, further comprising instructions that, when executed by the at least one processor, cause the system to determine the one or more cluster metrics by determining one or more of:
a cluster intensity metric corresponding to the cluster of oligonucleotides;
a cluster gain metric indicating a difference between a first signal intensity emitted from the cluster of oligonucleotides in a luminescent state and a second light intensity emitted from the cluster of oligonucleotides in a non-luminescent state;
a cluster offset metric indicating a signal intensity corresponding to the cluster of oligonucleotides in a non-luminescent state;
a signal-to-noise ratio (SNR) differential metric indicating a difference between an SNR for the first nucleotide read and an SNR for the second nucleotide read;
a guanine-cytosine (GC) content metric indicating an amount of sequenced nucleotide bases within the first nucleotide read or the second nucleotide read that include a guanine base or a cytosine base;
a phasing metric indicating phasing or pre-phasing of oligonucleotides within the cluster of oligonucleotides;
a nucleobase content metric indicating amounts of sequenced nucleotide bases within the first nucleotide read or the second nucleotide read that are adenine, cytosine, guanine, or thymine bases;
a polyclonality metric indicating a probability that the cluster of oligonucleotides includes oligonucleotides from two or more genomic samples;
a homopolymer content metric indicating an amount of homopolymer content within the cluster of oligonucleotides;
a cluster size metric indicating a size of the cluster of oligonucleotides within a sequencing image;
a relative cluster offset metric indicating a difference in signal intensity emitted from the cluster of oligonucleotides compared to an average signal intensity for a sequencing well in which the cluster of oligonucleotides is located;
an overlap metric indicating a number of overlapping nucleobases in a shared sequence between the first nucleotide read and the second nucleotide read;
an SNR metric indicating a signal-to-noise ratio at one or more of a portion of the first nucleotide read or a portion of the second nucleotide read;
a base call quality metric indicating a quality of a nucleobase call at one or more of a portion of the first nucleotide read or a portion of the second nucleotide read;
a cluster position metric indicating a position of the cluster of oligonucleotides within a region of a nucleotide-sample slide;
a region position metric indicating a position of the region within the nucleotide-sample slide; or
a free energy metric indicating an amount of energy to fold a molecule to a lower energy state.

10. A non-transitory computer readable medium storing instructions that, when executed by at least one processor, cause a system to:
identify, for a cluster of oligonucleotides corresponding to a sample genomic sequence for a genomic sample, a nucleotide read pair comprising a first nucleotide read complementing a first portion of the sample genomic sequence and a second nucleotide read complementing a second portion of the sample genomic sequence;
determine one or more cluster metrics associated with the cluster of oligonucleotides;
generate, using an insert length prediction model to process the one or more cluster metrics, at least one predicted insert length of the sample genomic sequence; and
determine a genotype call for a genomic coordinate within the sample genomic sequence based on the at least one predicted insert length.

11. The non-transitory computer readable medium of claim 10, further storing instructions that, when executed by the at least one processor, cause the system to determine the one or more cluster metrics by determining one or more overlapping nucleotide reads from different clusters of oligonucleotides that include nucleotide reads that map to a genomic region of the sample genomic sequence.

12. The non-transitory computer readable medium of claim 10, further storing instructions that, when executed by the at least one processor, cause the system to map the first nucleotide read and the second nucleotide read to a genomic region of a reference genome based on the at least one predicted insert length and nucleobase similarity between the genomic region and the first and second nucleotide reads.

13. The non-transitory computer readable medium of claim 10, further storing instructions that, when executed by the at least one processor, cause the system to generate the at least one predicted insert length by:
determining a length of a repeating sequence within a tandem repeat region of the sample genomic sequence; and
predicting a number of nucleobases for the at least one predicted insert length based on the length of the repeating sequence within the tandem repeat region.

14. The non-transitory computer readable medium of claim 10, further storing instructions that, when executed by the at least one processor, cause the system to determine the genotype call for the genomic coordinate by determining that the sample genomic sequence includes a structural variant based on comparing the at least one predicted insert length with an expected insert length for the sample genomic sequence.

15. The non-transitory computer readable medium of claim 10, further storing instructions that, when executed by the at least one processor, cause the system to determine the genotype call for the genomic coordinate by determining a tandem repeat corresponding to the genomic coordinate.

16. The non-transitory computer readable medium of claim 15, further storing instructions that, when executed by the at least one processor, cause the system to determine a repeat count of a repeat unit within a variable number tandem repeat (VNTR) by:
determining, for a set of haplotypes corresponding to the genomic coordinate, respective haplotype probabilities for the genomic sample at the genomic coordinate based on a set of nucleotide read pairs of the genomic sample corresponding to the genomic coordinate and a set of predicted insert lengths for the set of nucleotide read pairs;
selecting a highest haplotype probability from among the respective haplotype probabilities of the set of haplotypes; and
determining the repeat count of the repeat unit based on a haplotype length indicated by the highest haplotype probability.

17. A computer-implemented method comprising:
identifying, for a cluster of oligonucleotides corresponding to a sample genomic sequence for a genomic sample, a nucleotide read pair comprising a first nucleotide read complementing a first portion of the sample genomic sequence and a second nucleotide read complementing a second portion of the sample genomic sequence;
determining one or more cluster metrics associated with the cluster of oligonucleotides;
generating, using an insert length prediction model to process the one or more cluster metrics, at least one predicted insert length of the sample genomic sequence; and
determining a genotype call for a genomic coordinate within the sample genomic sequence based on the at least one predicted insert length.

18. The computer-implemented method of claim 17, further comprising:
determining that the at least one predicted insert length differs by a threshold number of nucleobases from an expected insert length for the sample genomic sequence; and
selecting, based on the at least one predicted insert length differing by the threshold number of nucleobases from the expected insert length, the genomic coordinate as a candidate genomic coordinate for structural variant calling.

19. The computer-implemented method of claim 17, further comprising:
identifying, for the genomic coordinate, a set of candidate alleles comprising a first candidate allele corresponding to the at least one predicted insert length and a second candidate allele inconsistent with the at least one predicted insert length; and
determining the genotype call for the genomic coordinate by determining a structural variant call or other genotype call corresponding to the genomic coordinate based on a comparison of the first candidate allele and the second candidate allele.

20. The computer-implemented method of claim 17, further comprising:
identifying, for the genomic coordinate, a set of nucleotide read pairs of the genomic sample and a set of candidate alleles comprising a first candidate allele corresponding to the at least one predicted insert length and a second candidate allele inconsistent with the at least one predicted insert length;
determining, for the set of candidate alleles, nucleotide-read fragment probabilities reflecting likelihoods of nucleotide-read fragments from the set of nucleotide read pairs supporting respective candidate alleles from among the set of candidate alleles;
identifying, from among the set of candidate alleles, the first candidate allele or the second candidate allele having a highest nucleotide-read fragment probability; and
determining the genotype call for the genomic coordinate by determining a structural variant call or other genotype call corresponding to the genomic coordinate based on the first candidate allele or the second candidate allele having the highest nucleotide-read fragment probability.
